(19) United States
(12) Patent Application Publication
(10) Pub. No.: US 2023/0169882 A1
(43) Pub. Date: Jun. 1, 2023

(54) SYSTEM AND METHOD FOR VIRTUAL MENTAL HEALTH SYSTEM INFRASTRUCTURE

(71) Applicant: Dr Egg Pty Limited, Woollahra (AU)

(72) Inventor: Gary Galambos, Woollahra (AU)

(21) Appl. No.: 17/819,428

(22) Filed: Aug. 12, 2022

(30) Foreign Application Priority Data
Aug. 13, 2021 (AU) 2021902531

(51) Int. Cl. G09B 19/00; G16H 20/70

(52) U.S. Cl. CPC G09B 19/00; G16H 20/70; G09B 5/02

(57) ABSTRACT

Systems may include a security system, one or more servers, and a platform for providing a plurality of digital mental health services to a user device. The digital mental health services may include one or more of eLEARNING service, CONNECT services, and PLUS other additional services that working together function as a “virtual mental health system infrastructure.”

### CROSS-REFERENCE TO RELATED APPLICATIONS

Pursuant to 35 U.S.C. § 119(a), this application claims the benefit of the filing date of Australian Patent Application Serial No. AU2021902531, filed Aug. 13, 2021, for “SYSTEM AND METHOD FOR VIRTUAL MENTAL HEALTH SYSTEM INFRASTRUCTURE,” the disclosure of which is hereby incorporated herein in its entirety by this reference.

### TECHNICAL FIELD

The present disclosure may relate to a system and method for providing virtual Mental Health System Infrastructure. More particularly, the present disclosure may relate to a system and method providing virtual Mental Health System Infrastructure adapted to educate, refer, and support patients to specialist-grade digital mental health services.

### BACKGROUND

Previously, mental health systems can only provide awareness information or limited support to patients or target very narrowly at some specific disorder.

US Patent Application No 2007/0299694 discloses a patient education management database system. The patient education system includes a server computer connecting to a wireless tablet computer. A database on the server includes a number of predetermined “presentation prescriptions,” each of which is a listing of educational sub-topics selected to provide an overview of a medical condition for patient education purposes. Each sub-topic has a corresponding content file (e.g., film clip) stored on the tablet computer. In operation, one of the default prescriptions is selected for providing educational content relevant to a patient's medical condition. Some of the sub-topics may be removed for customizing the default prescription. The tablet computer is given to the patient, and an educational presentation is displayed according to the presentation prescription, e.g., the content files that correspond to the topics in the selected presentation prescription are sequentially displayed. The patient's progress in viewing the presentation may be tracked, including the patient's answers to any questions in the presentation. However, traditional educational contents are not suitable for assisting the psychological and psychiatric patients or helpers. In particular, such a system is not designed for individual psychological and psychiatric patients or helpers.

U.S. Pat. No. 7,593,967 discloses a clinical reference and education system. The system is designed to provide medical condition diagnosis information that is gathered, stored, and distributed in the system database. More specifically, information regarding clinical/pathological differential analyses, key facts, clinical presentations, pathology features, imaging findings, anatomy information, medical references with abstracts, expert imaging center information, continuing medical education information, and related data is made available in electronic and printed forms via a general infrastructure of the system. These educational contents are not suitable for assisting the psychological and psychiatric patients or helpers. In particular, such a system is not designed for individual psychological and psychiatric patients or helpers.

U.S. Pat. No. 7,630,947 discloses a medical ontology information system for mining and/or probabilistic modelling. A domain knowledge base was created by a processor from a medical ontology. The domain knowledge base storing a list of disease-associated terms is used to mine for corresponding information from a medical record. The relationship of different terms with respect to a disease may be used to train a probabilistic model. The probabilities of a disease or chance of indicating the disease are determined based on the terms from a medical ontology. This probabilistic reasoning is learned with a machine from ontology information and a training data set. However, this knowledge base is not suitable for assisting the psychological and psychiatric patients or helpers. There is no user friend method to deliver such information to the stakeholders. In particular, such a system is not designed for individual psychological and psychiatric patients or helpers.

There is a lack of an online specialist-grade, trans-diagnostic and quality-controlled ‘mental health literacy’ service designed for patients to be directed to by their treating clinicians that can provide them with ‘self-help’ education (‘homework’) supplemented with ‘clinical support’ in between consultations. An effective digital mental health service could improve self-care, management coordination, resilience, and confidence through knowledge acquisition and improved decision-making and coping strategies (including help-seeking)—thereby improving ‘mental health literacy.’

Both primary care and specialist clinicians would appreciate the availability of a trustworthy, competent, user-friendly, and engaging ‘virtual therapeutic space’ to direct their patients to, which could ‘hold,’ ‘contain,’ support, and, guide their patients—keeping them occupied—between consults. That space would be valued by the clinicians if their patients returned to them more knowledgeable about their mental health, more skilled at coping with their mental ill-health and having more understanding about the skillsets of their clinicians (and other type of clinicians) and thereby having deeper understanding about how they can optimally utilise their existing clinicians (and other types of mental health clinicians) and other providers. Trust in the platform would be enhanced if it was designed and maintained by mental health clinicians led by psychiatrists informed by healthcare principles, values, and ethics, and operated according to the standard processes of psychiatric hospitals and community healthcare facilities.

Currently, there are no specialist-grade virtual hospital-community healthcare system models using digital health platforms and Telehealth to provide assistance to health funders paying for healthcare, healthcare-related and income-replacement payments to consumers with mental disorders where there is overlapping maladaptive and inappropriate help-seeking behaviors and unhealthy dependencies with the healthcare providers.

There is an absence of specialist psychiatry-grade broad scope, pragmatic and clinically-oriented eLEARNING service for improving mental health outcomes, recovery from mental disorders, reducing disability, and improving resilience and mental fitness, that is highly engaging (maintaining user interest to achieve high usage or adherence rates), understandable (uses layman's language) and pragmatic (provides applicable, transferable knowledge, skills and strategies), and suitable for all types of users.

There is an absence in the prior art of a mental health consumer-provider brokerage service that uses clinical care pathways for the education, training, and accreditation, and provision of ‘clinical support’ services, which has an underlying virtual hospital infrastructure to ensure minimal standards and quality control, and that utilizes contemporary psychiatric care and practice models.

There is a lack of digital health services available to general practitioners and specialists to refer their patients to cutting-edge digital mental health services delivering online interventions such as Artificial Intelligence, Virtual Reality, and remote access to stimulation therapies to supplement and complement the current care being received.

Any discussion of the prior art throughout the specification should in no way be considered as an admission that such prior art is widely known or forms part of the common general knowledge in the field.

### BRIEF SUMMARY

It may be advantageous to provide a system comprising a security system, one or more servers, and a platform for providing a plurality of digital mental health services to a user device, wherein the digital mental health services comprising one or more of eLEARNING service, user-individual mental healthcare provider brokerage or matching services, and other additional mental healthcare service provider, which, working together, function as a ‘virtual hospital and mental health system infrastructure.

Preferably, the digital mental health services comprise eMental Health Literacy Education, Training, and Accreditation.

Preferably, the eLEARNING service is adapted to provide all user types in the one product, including self-help, help-seeking, and peer support mentor consumers; mentor, clinician, and Continuing Professional Development (CPD) services:

Preferably, the eLEARNING service incorporates autonomous learning principles with an innovative true visual learning approach with visual analogies, role play with conversing characters and avatars enabling a minimization use of technical words and jargon.

Preferably, the eLEARNING service incorporates continuous user interactivity, personalizing opportunities and embedded quizzes to ameliorate training pressure.

Preferably, the eLEARNING service incorporates and integrates innovative psychiatry-grade ‘categorizations’ of all different explanatory models (including sophisticated biopsychosocial models), approaches, treatment modalities and syllabus to complement and enhance recognized existing descriptive classifications or categorizations. Preferably, the eLEARNING service breaks down the integrated specialist-level classifications and categorizations into simple language and building blocks.

Preferably, the eLEARNING service is adapted to supplement, complement, and make more efficient and safer existing in-person GP, specialist, and allied health mental health care.

Preferably, the system further comprises an artificial intelligent (AI) engine for processing user feedback from the eLEARNING service and adjust an eLEARNING program based on the user feedback and the aggregated data of other users.

Preferably, the digital mental health services are adapted to provide Online access for supplementary and complementary services to existing GP and specialist care that provide access to cutting-edge interventions and technological developments, such as artificial intelligence, Virtual Reality and neurostimulation therapies.

Preferably, the user device comprises a processor, a human user interface, a machine-to-machine interface and a network device, herein the human user interface is adapted to deliver a product generated by the AI engine, the human user interface is adapted to deliver a virtual reality environment, and the machine-to-machine interface is adapted to connect to a medical device to deliver stimulation therapies.

Preferably, the network device is adapted to connect to the digital mental health services where telecommunication health consultation services can be delivered.

Preferably, the virtual reality environment comprises a Virtual Hospital-Community Mental Health Care environment with a specialized psychiatric online healthcare system infrastructure.

Preferably, the specialized psychiatric online healthcare system infrastructure is adapted to provide clinical care pathways that are supplementary and complementary to existing in-person care, incorporating multi-modal, trans-diagnostic, multi-user type services, and has hospital-grade healthcare standards, regulations, and accreditation processes to maintain quality control for all users.

In the context of the present disclosure, the words “comprise,” “comprising” and the like are to be construed in their inclusive, as opposed to their exclusive, sense, that is in the sense of “including, but not limited to.”

The present disclosure is to be interpreted with reference to at least one of the technical problems described or affiliated with the background art. The present aims to solve or ameliorate at least one of the technical problems and this may result in one or more advantageous effects as defined by this specification and described in detail with reference to the preferred embodiments of the present disclosure.

### BRIEF DESCRIPTION OF THE DRAWINGS

To easily identify the discussion of any particular element or act, the most significant digit or digits in a reference number refer to the figure number in which that element is first introduced. The patent or application file contains at least one drawing executed in color. Specifically, FIGS. 2 through 60 of the patent or application file are executed in color. Copies of this patent or patent application publication with color drawing(s) will be provided by the Office upon request and payment of the necessary fee.

FIG. 1 is a schematic diagram of a multi-purpose platform system for digital mental health services according to one embodiment;

FIG. 2 is a schematic diagram of a virtual mental health system infrastructure of the system of FIG. 1;

FIG. 3 is a schematic diagram of a performance wave (a visual analogy of certain mental functions or symptoms) depicting a service provided by the system of FIG. 1;

FIG. 4 is a schematic diagram of an eLEARNING Ship Training Unit provided by the system of FIG. 1;

FIG. 5 is a narrative of a visual learning method provided by the system of FIG. 1;

FIG. 6 is another narrative of a visual learning method provided by the system of FIG. 1;

FIG. 7 is another narrative of a visual learning method provided by the system of FIG. 1;

FIG. 8 is another narrative of a visual learning method provided by the system of FIG. 1;

FIG. 9 is another narrative of a visual learning method provided by the system of FIG. 1;

FIG. 10 is another narrative of a visual learning method provided by the system of FIG. 1;

FIG. 11 is another narrative of a visual learning method provided by the system of FIG. 1;

FIG. 12 is another narrative of a visual learning method provided by the system of FIG. 1;

FIG. 13 is another narrative of a visual learning method provided by the system of FIG. 1;

FIG. 14 is another narrative of a visual learning method provided by the system of FIG. 1;

FIG. 15 is another narrative of a visual learning method provided by the system of FIG. 1;

FIG. 16 is another narrative of a visual learning method provided by the system of FIG. 1;

FIG. 17 is another narrative of a visual learning method provided by the system of FIG. 1;

FIG. 18 is another narrative of a visual learning method provided by the system of FIG. 1;

FIG. 19 is a diagram of a tug boat interface provided by the system of FIG. 1;

FIG. 20 is a diagram describing the packages provided by the system of FIG. 1;

FIG. 21 is a diagram describing the packages provided by another embodiment of the system of FIG. 1;

FIG. 22 is a schematic diagram of an eLEARNING Ship image depicting mental disorders on its sails for easy user access provided by the system of FIG. 1;

FIG. 23 is a schematic diagram of the sail path of the user's ship that has been utilized as a visual analogy for the eLearning program;

FIG. 24 is a schematic diagram of a rectangular aperture provided by the system of FIG. 1

FIG. 25 is a schematic diagram of another eLEARNING Ship image provided by the system of FIG. 1;

FIG. 26 is a schematic diagram of another version of the eLEARNING Ship image provided by the system of FIG. 1;

FIG. 27 is a draft home page display on a user device of an embodiment of the present disclosure;

FIG. 28 is a schematic diagram of another interface provided by the system of FIG. 1;

FIG. 29 is a schematic diagram of another interface provided by the system of FIG. 1;

FIG. 30 is a schematic diagram of another interface provided by the system of FIG. 1,

FIG. 31 is a schematic diagram of another interface provided by the system of FIG. 1;

FIG. 32 is a schematic diagram of another interface provided by the system of FIG. 1;

FIG. 33 is a schematic diagram of another interface provided by the system of FIG. 1;

FIG. 34 is a schematic diagram of another interface provided by the system of FIG. 1;

FIG. 35 is a schematic diagram of another interface provided by the system of FIG. 1;

FIG. 36 is a schematic diagram of another interface provided by the system of FIG. 1;

FIG. 37 is a schematic diagram of another interface provided by the system of FIG. 1;

FIG. 38 is a schematic diagram of another interface provided by the system of FIG. 1;

FIG. 39 is a schematic diagram of another interface provided by the system of FIG. 1,

FIG. 40 is a schematic diagram of another interface provided by the system of FIG. 1;

FIG. 41 is a schematic diagram of another interface provided by the system of FIG. 1,

FIG. 42 is a schematic diagram of another interface provided by the system of FIG. 1;

FIG. 43 is a schematic diagram of another interface provided by the system of FIG. 1;

FIG. 44 is a high-level schematic diagram of the system of FIG. 1;

FIG. 45 is a schematic diagram of a site map of the system of FIG. 1;

FIG. 46 is a schematic diagram of interfaces of the system of FIG. 1;

FIG. 47 is a schematic diagram of another interface of the system of FIG. 1;

FIG. 48 is a schematic diagram of an interface showing a three-stage journey provided by the system of FIG. 1;

FIG. 49 is a schematic diagram of another interface showing a three-stage journey provided by the system of FIG. 1;

FIG. 50 is a schematic diagram showing three user groups of the system of FIG. 1;

FIG. 51 is a schematic diagram showing roles of the help provider groups of the system of FIG. 1;

FIG. 52 is a schematic diagram describing the role of the mentor help provider group of the system of FIG. 1;

FIG. 53 is a schematic diagram of describing the role of the clinician help provider group of the system of FIG. 1;

FIG. 54 is a schematic diagram describing the roles of the coach help provider group of the system of FIG. 1;

FIG. 55 is a schematic diagram showing an interface of the help provider groups of the system of FIG. 1;

FIG. 56 is a schematic diagram showing 3 categories of gaps in digital mental health services provided by the system of FIG. 1;

FIG. 57 is a schematic diagram showing person-centered care model features provided by the system of FIG. 1;

FIG. 58 is a schematic diagram of a part of an artificial intelligent engine of the system of FIG. 1;

FIG. 59 is a schematic diagram of another part of an artificial intelligent engine of the system of FIG. 1; and

FIG. 60 is a schematic diagram depicting the MANAGEMENT LINKS where the choices the user has made to explore management options during their journey through the subunit is linked by CONNECT and PLUS services to the database of accredited mental health providers and digital mental health services. Its capacity for data matching brings together the 3 services (eLEARNING, CONNECT and PLUS) for the user who wants to enhance their education by using additional services.

### DETAILED DESCRIPTION

The present disclosure is a multi-purpose platform system 10 (also referred to as “MindSkiller”) containing a suite of digital mental health services 20 (eLEARNING service, CONNECT service, and PLUS additional services) that working together function as a ‘virtual hospital and mental health system infrastructure.’ It provides quality control similar to psychiatric hospitals around what happens in relation to specific types of mental health care.

In one embodiment, the system is adapted to provide a virtual hospital and virtual community of consumers and providers, creating a mental health ecosystem. The method of the present disclosure is adapted to use a unique mental health categorization system that incorporates the psychiatry-grade biopsychosocial model and all other contemporary specialist healthcare models, which ‘breaks’ the field of mental health into ‘building blocks.’ In one embodiment, the system and method of the present disclosure is adapted to create a foundation level infrastructure for the virtual hospital and virtual community.

The type of support facilitated by the CONNECT service is innovative in being user-determined Telehealth-based episodes of psychoeducational guidance based on the user's needs in the moment whilst undertaking the mental health literacy program. The design is for the user to experience episode durations under 30 minutes that are intended to occur on multiple occasions whilst they are working through the eLEARNING content. This episode pattern contrasts to the typical psychotherapy model of care where consultations typically have durations that are predetermined by the provider and are 30-60 minutes long. This Telehealth-based method of mental health literacy support and guidance thereby expands and enhances traditional mental health care models.

Current, existing clinical reference and education systems about medical conditions and diagnoses are not designed and cannot simply be used to provide pragmatically useful information about mental disorders for the following reasons:
1) Mental disorders tend to come in multiples, where a young patient tends to be diagnosable with anywhere between one and five disorders, with the average being two or three diagnoses rather than one diagnosis. The disorders not only often have common risk factors but influence each other such that one disorder may become a risk factor for other disorders. Hence, there may be a hierarchy, and the responsiveness of a mental disorder to treatment may be influenced by the presence of another disorder.
2) Most general medical disorders are diagnosed using investigations whereas most mental disorders are diagnosed using clinical assessment of symptoms, signs and mental state. Efficient computer-based systems have not been generally implemented due to the excessive time it takes to use such methods for the purposes of diagnosis-making in an individual.
3) Classification using the standard classification systems (DSM-5 and ICD-11) provide a descriptive classification based on symptom checklists only. There is no consideration of aetiology like most general medical disorders. When those descriptive classification systems were first designed, there was an assumption that diagnostic formulations would be used to complement them, which take into account:
   a) predisposing, protective and propagating factors (triggers);
   b) an aetiological system known as the biopsychosocial model, which examines the contribution of biological, psychological and social factors as the mechanism of the disorder;
   c) personality assessment based on a dimensional rather than a categorical model;
   d) other treatment modality-informed or guided models such as insight-oriented psychodynamic (interpretive or psychoanalytic), cognitive and behavioral models;
   e) neuroscience-based models have also contributed to formulating mental health problems particularly with regard to specific mental disorders. There has also been a resurgent interest in evolutionary models to explain the mechanisms of mental disorders regarding physiological, genetic and behavioral tendencies having evolved according to natural selection also creating disorder susceptibilities.
4) The non-descriptive elements and models have not been appropriately utilized for educating consumers or other mental health clinicians because:
   a) they have become less utilized over time by clinicians because clinicians have preferred to use the descriptive classifications, which are a seductively simple and succinct method of categorizing the mental health problems of patients, even though they fail to take into account the explanatory aspects of most presentations;
   b) these are highly complex concepts challenging to explain to non-specialist trained clinicians or consumers without the development of educative models that simplify the communication and learning processes. This has been undertaken in MindSkiller eLEARNING in an embodiment of the present disclosure by providing a simple categorization approach to both the descriptive and non-descriptive elements of mental disorders and mental health in general.

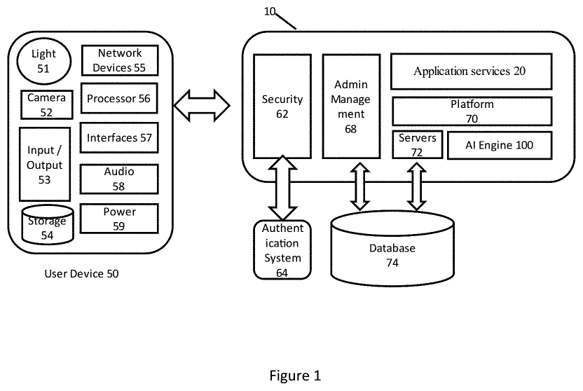

In one embodiment, the system 10 as shown in FIG. 1 comprises platform 70 running a number of digital mental health services 20 (e.g., provided via one or more software applications) on a plurality of servers 72. The servers 72 can provide redundancy to the system 10 such that it will avoid any downtime or interruption of the digital mental health services 20 to users. In one embodiment, it is important to maintain continuous and uninterrupted digital mental health services 20 because bad user experience may affect the efficiency and effectiveness of the MindSkiller system 10. In a preferred embodiment, the system 10 comprises an artificial intelligent engine 100 is provide a personalized and anthropomorphized program for stakeholders in the mental health area including patient, clinician, doctors, nurses, case managers, and other helpers. The system 10 is preferably maintained its own database 74 for storing user records and activities. Due to the complexity of the system 10, it is preferable to have an administration management system 68 for technical experts to manage the infrastructure of the MindSkiller system 10. Preferably, the administration management system 68 is adapted to provide a separate credential to the technical expert such that they may have access to the infrastructure of the system 10 while not having access to individual user records such as patient records. Preferably, the system 10 comprises a security system 62 to keep track security of the system including generating, keeping, and maintaining the credential of different users. In one embodiment, the security system 62 is adapted to connect to a separated authentication system 64 for verifying and validating the identity of a user. This may involve biometric or two factors authentication. The authentication system 64 may generate a credential of a user and send it back to the security system 62. The security system 62 may also be responsible for authorizing certain actions or events of a user based on the credential given to a user.

In one embodiment, the system 10 is adapted to connect to a user device 50 to deliver the digital mental health services 20 to users. The user device 50 comprising a processor 56 for processing data of the device and ensure quick response time. The user device 50 may have different human user interfaces, such as video camera 52, audio system 58, and I/O devices 53. The I/O device 53 may be a touch screen, or a display and a keypad. The user device 50 may have one or more light sources 51 to assist the video camera 52 or imaging sensors to capture the image of a user. The light sources 51 may emit visual light, infra-red light, or light amplification by stimulated emission of radiation. In addition to normal photograph image and video, the user device 50 may help to capture different types of imagery of the user such as remote photoplethysmography, and transdermal optical imaging, etc. The user device 50 comprises network device 55, which is adapted to allow the user device to communicate with other devices using one or more network protocols such as Internet Protocol, 4G/5G, BLUETOOTH®, etc. It comprises a plurality of networking stacks for sending and receiving messages, which can relieve much of the overload to the processor 56. In one embodiment, the network device 55 is adapted to handle data encoded in HL7. In one embodiment, the user device 50 comprises interface 57 for connecting to different devices. The interface 57 can be a USB connector or RS525 connector, etc. It allows the user devices to connect directly to medical equipment, such as electroencephalogram electrodes or monitor, or other wetware. The user device 50 comprises its own data storage 54 for storing local data and settings. The user device 50 preferably comprises its own power source 59 such as it can be portable.

The platform provides mental health literacy education and training through the eLEARNING service, clinical support through the CONNECT service, and access to innovative digital mental health services through the PLUS service to referred patients, sole individuals, and the full spectrum of help-providers including all types of primary care and specialist medical and non-medical clinicians, as well as mentors (peer supports, family and careers, case managers) and coaches. It also can provide clinical practice enhancement, clinician CPD, mental health system enhancement, and organizational mental health support and enhancement.

In one embodiment, the system 10 of the present disclosure includes:

1) Clinicians' Aide clinical practice enhancement services, which can seamlessly support, assist, supplement, complement and enhance standard outpatient and/or inpatient mental health care delivered face-to-face or by Telehealth, by any healthcare provider but particularly aimed at mental health clinicians such as general practitioners, psychiatrists and psychologists.

The system of the present disclosure can generate and maintain a ‘virtual therapeutic space’ for clinicians to refer their patients to, which will ‘hold,’ ‘contain,’ support and guide them in between consults. It is a specialist-grade psychiatric digital system that can partially fill gaps and delays in accessing specialist-level psychiatric services with an effective, quality, efficient and user-friendly services that supplement, complement and enhance primary care. It will assist the general practitioners, psychologists and other primary care level healthcare providers to support their patients whilst they are waiting for assessment, management plan development and intervention by specialist psychiatric services or consultant psychiatrists.

The components of the system of a preferred embodiment that can achieve Clinicians' Aide clinical practice enhancement are:

(i) Mental Health Literacy education through the eLEARNING service—a psychiatry-grade broad-scope, pragmatic clinically-oriented user-centered course for improving mental health outcomes, assisting recovery from mental disorders, reducing psychopathology and improving resilience and mental fitness, that is highly engaging, comprehendible and utilizable as it is designed to utilize autonomous learning principles, maintaining user interest to achieve high completion rates and positive health outcomes, and providing Progress Reports to referrers if the user consents. Referring help-providers obtain access to the educative material, which they can use in their practices to facilitate their mental health consultations and care of their patients.

The “user-centered course” referring to in this specification is broader in function than a ‘course.’ It has various functions including being:
a) a mental health ‘course’ for users wishing to complete a mental health literacy program designed to improve their awareness about the clinical field of mental health both in general and in regards to the common categories of mental disorders and psychiatric diagnoses more specifically. The course is purposefully clinically-oriented and non-academic in perspective, but developed from an evidence-based specialist psychiatrist viewpoint. To recognize the knowledge and skills development, certification is gained from completing minimal requirements, for those who wish to achieve that formal recognition.
b) a mental health ‘encyclopedia’ for users wishing to examine particular areas of interest (transdiagnostic and specific mental disorders) to expand their knowledge and skills, but in a non-sequential, self-directed and tangential manner.
c) a mental health individual help-provider matching ‘search engine’ providing users with Telehealth-accessible specific help-providers (clinicians, mentors and coaches).
d) a digital mental health service matching ‘search engine’ providing users with online access to specific digital mental health services, which also encourages and facilitates innovation in mental health services that improves mental health literacy, support and clinical interventions.
e) a knowledge-matching ‘search engine’ providing users with online access to specific mental health-related information.
f) serving to provide the foundations and building blocks of virtual hospital and community mental health healthcare delivery services.

(ii) Real-time Telehealth ‘Clinical Support’ (multi-channel, including video conferencing) by three types of Accredited Help-Providers (mentors, medical and non-medical clinicians and coaches) from within the eLEARNING service that provides defined brief episodes of support and guidance, giving the users a ‘taste’ of what types of knowledge, skillsets and interventions those types of Help-Providers can offer, and giving the consumer and their external clinicians some experiential understanding about what management plan components may be useful to pursue (personalizing, enhancing and advancing care) thereby improving outcomes; and

(iii) Innovative Digital Mental Health Services that align with the system 10 or platform of an embodiment of the present disclosure and its healthcare content, goals and values—available to general practitioners and specialists to refer their patient to cutting-edge novel digital mental health services delivering online interventions such as artificial intelligence, virtual reality and remote access to neurostimulation therapies to supplement and complement the current care being received.

2) Standalone Mental Health Literacy Education eLEARNING “Self-Help” services—a psychiatry-grade broad-scope, pragmatic clinically-oriented course for solitary consumers (independent ‘Help-Seekers’) who have not yet engaged with any external mental health providers but wish to understand their mental health, their disorders, the common causes for their disorders, explore what types of help-providers there are available, what types of interventions are available that may be useful in their circumstances and also learning about self-help skills and strategies that they can utilize.

The ‘self-help’ education of the present disclosure is different to that of the cognitive behavior therapy (CBT) programs that are very narrow problem-solving or symptom-focused and narrow modality interventions. The CBT programs provide narrow-focused behavior therapy and cognitive reframing. The differentiation of the educational aspect of the present disclosure is that ‘self-help’ education and homework of an embodiment of the present disclosure is not narrow in modality nor type of problem being targeted. The homework of the present disclosure is directed at what happens in the real-world—where people present to their clinicians with more than one disorder or problem and in practice need multiple modalities to be used. Rather than homework, it is self-directed, autonomous e-learning that the user engages in, in between consults/meetings with their clinicians, patients or help-seekers. The homework of the present disclosure is adapted to teach using an unorthodox innovative approach more akin to learning a foreign language—using an infotainment styled immersive apprenticeship type experiential method in conjunction with the use of a categorization system applied to explanatory models to complement the contemporary classification systems and treatment approaches.

3) Standalone Mental Health Literacy Training eLEARNING “Help-Provider” accreditation support services—psychiatry-grade broad-scope, pragmatic clinically-oriented basic and advanced training for help-providers (mentor, clinician—medical and non-medical—and coach) types of help-provider wishing to gain mental health literacy training and accreditation that will:

(i) enable them to provide online ‘clinical support’ to help-seekers using the platform or system of an embodiment of the present disclosure (the CONNECT service utility that works in conjunction with the eLEARNING utility);

(ii) assist to upgrade their skillsets in their own external practices and workplaces; and

(iii) gain Continuing Professional Development (CPD) points or credits toward their registration bodies' and professional associations' or colleges' CPD programs.

4) ‘Virtual Hospital’ Support to Subscription Organizations service that provides a novel quality healthcare, efficient and cost-effective solution to managing high-needs employees, members and insurees (of corporations and health insurance companies e.g., private health insurers, workers compensation insurers and life insurers) with mental disorders or presumed mental health comorbidity to their physical health problems that is preventing their achieving a functional recovery or reducing their productivity (e.g., as evidenced by number of sick days, underperformance, disruptive or disengaged behaviors). It is the only digital mental health service that incorporates all the contemporary specialist psychiatric models of care, healthcare principles and values, latest research and aligning with Australia's National Digital Health Strategy.

An ‘outsource model’ gives the following advantages to the subscribing organization:

(i) Being at arms-length from the service delivery provides ‘safety’ from legal liability and damage to reputation (e.g., claims of insensitivity, perceived bias, conflict of interest, misguided values).

(ii) Encourages members directed to complete the program to take their engagement more seriously than would otherwise be the case if it were in in-house program and instils hope that the supplementary support and assistance will likely complement their existing care rather than cause a split in their care, conflict or premature reduction in healthcare or financial support. If there are reductions in either healthcare or welfare, the consumer and their external personal and professional supports will likely be more attuned, supportive and less adversarial as the rationale will always be for what is in the best interests of the consumer's mental health as perceived not only by the consumer or any sole external help-provider, but by the entire multidisciplinary ‘team’ overseeing the consumer's management plan.

(iii) Assists with product validity due to it being seen to be and experienced as an adequately independent healthcare service.

(iv) Enables optimal credibility as the service will be a modified digital mental healthcare service that has been developed for the entire community for the purpose of improving healthcare outcomes as the platform is a specialist psychiatric online healthcare system infrastructure for the management of mental disorders, increasing mental health resilience and improving metal fitness. It does this by:

(a) providing clinical care pathways that form a framework that is constructed from contemporary healthcare models of care;

(b) being supplementary and complementary to existing care (face-to-face and telehealth delivery) and in scenarios where there are private healthcare funders (corporations, health insurance companies e.g., private health insurers, workers comp insurers and life insurers);

(c) incorporating content that is multi-modal, trans-diagnostic and applicable to multiple types of users;

(d) having hospital-grade healthcare standards, regulations and accreditation processes to maintain quality control for all users (consumers, mentors, clinicians, coaches and external consumer helpers);

(e) having a primary agenda of improving healthcare outcomes, which will reduce costs. Reducing healthcare and welfare costs is an important—but secondary—benefit of the service;

(f) improved healthcare outcomes including the health concept of maximizing an individual's independence, capacity for taking responsibility for their health, wellbeing, activities of daily living and choosing and engaging in personally meaningful activities that will contribute to a good quality of life. It is recognized that many chronically disordered and disabled members are either purposefully or unintentionally self-sabotaging their outcomes as a result of personality disorders that lead to various types of behaviors that propagate their symptoms and loss of function and resistance to recovery, such as:

i. externalizing behaviors—highly self-defensive behaviors causing distress to contacts and gaining material and psychological benefits at others' expense (consciously or unconsciously) including secondary gain undermining recovery

ii. internalizing behaviors—bottling emotions and developing self-sabotaging belief systems and unhelpful habits, all leading to poor coping with their distress and undermining recovery

iii. being out of touch with reality—a detachment from reality with numbness or paranoid belief systems and a tendency to oscillate perspectives that undermines therapeutic engagement and also undermining recovery

iv. all of these above 3 groups are likely to have engaged in therapeutic relationships that are suboptimally assisting them to recover from their disorders and disabilities and may even be unintentionally contributing to undermine their recovery. That's because they all have tendencies to form unhealthy and unhelpful dependencies on others. They either have not developed the capacity for forming values toward others encouraging them to take responsibility adequately for themselves, do not have the assertiveness to depend more on themselves, or do not have the capacity to put enough trust in others/the external world to enable them to find a balanced, healthy level of dependency on others Vs themselves—enabling them to form more balanced relationships with others (i.e., healthy dependencies with their supports, healthcare providers and funders).

These consumer groups are usually the highest cost individuals to any healthcare system and healthcare funding or welfare support schemes. By definition, these members have poor decision-making and coping skills, which make them more vulnerable to develop multiple other mental disorders and physical health conditions, to have more severe and dramatic courses, and to be more treatment-resistant to remission.

Adequate management often requires a harm minimization approach, with external limit-setting and reality orientation, which is best delivered by a multidisciplinary team. Legal contractual and regulatory limits need to often be co-utilized to set limits and provide an arena within which healthcare can take place where opinions are collectively formed based on collaborative discussion of objective information, bigger picture and longitudinal perspectives, and realistic measures that often require negotiation and compromise that are beyond the consumer's individual capacity and possibly impeded by any unhealthy therapeutic relationships they may have fostered, formed and maintained.

Introducing specialist and allied health clinicians into the team is likely to over a period of time permits constructive decisions, resolutions and actions to be taken, will usually include reappraisal, rationalization and realistic assessment of what level of intervention and resources is reasonable for the individual when the objective is optimizing their independence and responsibility and minimizing unhealthy dependency and reinforcement of unhelpful belief systems and habits. Therefore, stepwise withdrawal of resources is a likely outcome that will mutually benefit the member and the funding organization that has found themselves ‘stuck’ with an indefinitely needy and demanding member.

If the member's funding is dependent on their participation in the ‘virtual hospital’ program, that will motivate them to continue to progress within the program, becoming more and more mental health literate, which may improve reflective capacity and cooperation with support and accepting the expectation that they become more resilient, competent and independent. Their participation and finally their capacity to function as a peer support help-provider will contribute to evidence of their functional improvement. All the functional improvements they make will add to the objective evidence that they are recovering and in less need of high resources and funding. The core strategy is to require the member to engage in a positively reinforcing process whereby their participation provides evidence of their functional improvement, facilitating a stepwise reduction of funding their disability.

Case Manager Credibility and Capacity to Mentor services provided by an embodiment of the present disclosure enables the development of improved credibility and capacity of the subscribing organization's case managers as bona fide mentors for the members as case managers will be required to complete the eLEARNING service to become accredited to assist member users and engage in case conferences, communication with external health providers and contribute to the management plan development. It may also be useful for the acculturation of the case managers through their engagement with a health healthcare service that has embedded into its service By-Laws specialist healthcare models, values and ethics.

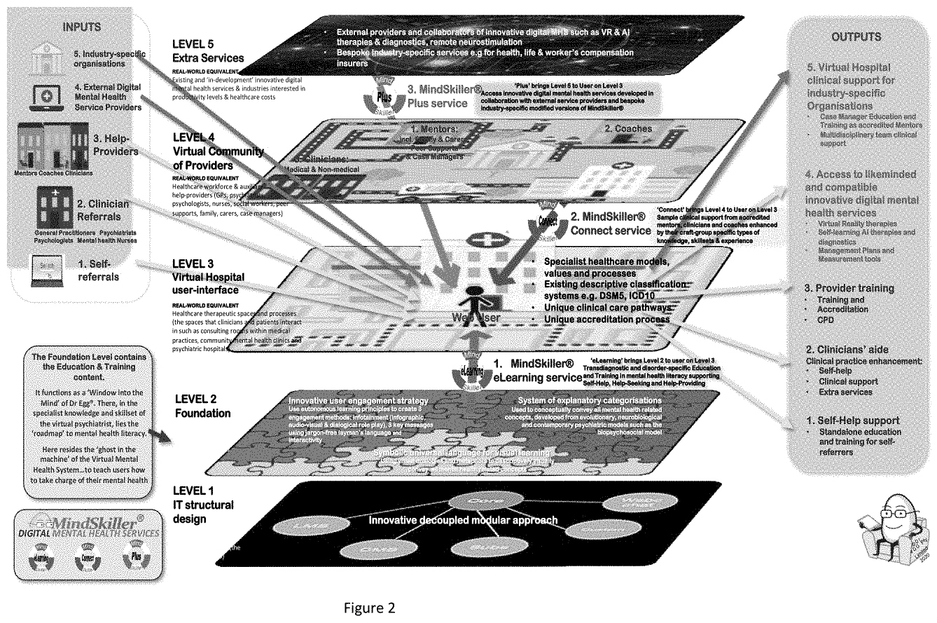

A seamlessly integrated virtual mental health system of the present disclosure is designed by ‘reverse-engineer’ the ‘parts’ or ‘components’ or ‘building blocks’ of the underlying virtual infrastructure, most of which have needed to be ‘built from scratch’ (invented—see below design levels 1, 2, 4 and 5) and the other parts (in level 3) have needed to be re-constructed in an innovative manner (synthesized, modified, translated) to work together cohesively (see level 3) as a system of ‘clinical care pathways’ from where the user can interact with other levels to access the services and functions they provide. As shown in FIG. 2, the virtual mental health system contains the following infrastructure components that conceptually match their external real-world equivalents:

This schematic of the ‘Virtual’ Mental Health System Infrastructure system or platform of an embodiment of the present disclosure can be demonstrated three processes:

Referring to FIG. 2, the is described from bottom-to-top contains 5 Levels of layered virtual infrastructure, in the form of an ‘architectural blueprint’ design of an electronic virtual mental health computer system of an embodiment of the present disclosure:
Level 1 IT structural design level (innovative decoupled modular
approach)
Level 2 Foundation level
Level 3 Virtual hospital user-interface level
Level 4 Virtual Community of Providers level
Level 5 Extra services

From left-to-right of FIG. 2, there is provided 5 inputs (of the different user groups) of the electronic virtual mental health computer system of an embodiment of the present disclosure:
1 Self-referrals
2 Clinician referrals
3 Help-Providers
4 External Digital Mental Health Service providers
5 industry-specific organizations

As shown in FIG. 2, there are 6 outputs (of the types of healthcare support the MINDSKILLER® platform can provide these user groups with:
1 Self-help support
2 clinical support
3 clinicians' aide
4 provider training
5 access to likeminded and compatible innovative digital mental health services
6 virtual hospital clinical support for industry-specific organizations)

Referring to FIG. 2, the system of the present disclosure, there is shown three dashed arrows showing how indicate how the three digital health services unique to the present disclosure—eLEARNING Services, CONNECT Services, and PLUS Services (or auxiliary Services)—brings to the web user shown here enable the web user on Level 3—the ‘Virtual Hospital’ user-interface interfacing level with of the platform—to access in the ‘Virtual Hospital’ of Level 3, the content of each of the 3 other resource Levels:
the eLEARNING education and training content of Level 2, the
virtual community of providers of Level 4 and the
extra services of Level 5
   (bespoke versions of the platform or system of the present disclosure—and
   other likeminded and compatible digital mental health services that have either independently developed content that is complementary or expands) of Level 5.
   digital mental health services that have been developed in collaboration with the system or platform of the present disclosure to expand its content.

Each of the levels are discussed in the following table, where the real-world equivalent is given adjacent to the virtual mental health system infrastructure component:

The key ingredient missing from the current digital healthcare systems is a specialist psychiatry-grade Education and Training system that incorporates trans-diagnostic as well as specific disorder and multi-modal treatment content that has a positive engagement strategy relying on its organization into a constellation of complementary visual analogies, new enhanced classificatory system of explanatory categories (to complement and enhance the existing descriptive disorder categories) and autonomous learning principles.

The MindSkiller mental health literacy Elearning course's key design strategies were:

(i) to embed technical terms and jargon within the content using a method that could hide, disguise and minimize any specialized language that an educated layman may not know from present pop-culture.

(ii) for the material to be highly engaging for the users (see ingredients below).

(iii) for the user's capacity to process, register and recall the information provided to be taken into account, such that there would be no more than three key messages at a time.

To achieve these novel design strategies, the ingredients of the approach developed includes:

(1) providing an exquisitely purist ‘visual learning’ infotainment methodology in the system where complementary and integrated ‘theme-based’ images and animations are served as core building blocks of the primary ‘symbolic language’ that the user learns experientially as they journey through the storylines. The images and animations are graphical direct representations of mental health concepts. The avatars' speech bubbles and sparse text supports the images rather than images supporting text (the usual status quo). Videos are only sparingly used for optional extra introductions to the units and are not necessary for the user to watch as they hold no unique information (which also differs from some of the educational digital mental health services currently available, which either rely predominantly on text or on videos as the core educational or training delivery methods). Clinician Providers will experience accelerated learning because of their pre-existing knowledge and skillsets and will find it refreshing to re-conceptualize their jargon-rich terminology of the mental health concepts into systems of visual representations and of simple explanatory categorizations that have embedded into their design contemporary psychiatric patient and system care models, principles, values and aspirations.

(2) Adopting ‘Autonomous learning’ principles similar to that used to develop narrow-targeted cognitive remediation programs for use in a broader-scale, comprehensive multi-dimensional learning system. The cognitive based programs are narrowly targeted toward either assisting people with chronic psychotic disorders or children with learning disorders like dyslexia and attention deficit hyperactivity disorder that employ visual learning and autonomic learning principles. These are simple skill-based learning exercises using neuropsychological cognitive-behavioral learning models aiming to strengthen or re-train specific underdeveloped neurological modalities in the case of childhood learning and developmental deficiencies and target so-called ‘negative symptoms’ in patients with chronic psychotic disorders, which also target improving injured neurological circuits that affect attention, motivation, social cognition and judgement in essential basic daily activities of living. Online cognitive training programs promising to improve various types of cognition like memory (IQ) have been developed such as Lumosity.com.

(3) Providing a theme-based ‘symbolic language’ of interconnecting representational visual analogies, metaphors and infotainment styled dramatic narratives employing role play interface in the system of the present disclosure. Most of the text is embedded in speech bubbles from conversations between characterological representations of three team members—a programmed avatar of the psychiatrist inventor, a programmed avatar assistant who represents either a mentor, clinician or a coach and the user is represented by an ‘alter ego’ in-theme avatar. The assistant avatar is a Tugboat with three life preservers, each of which is a clickable link to each of the three MindSkiller services eLEARNING, CONNECT and PLUS. MindSkiller CONNECT service will activate the brokerage service that enables the user to video communicate with a mentor, clinician or coach for brief episodes of clinical support whilst they are using the eLEARNING service. In future versions the user will be able to receive that support and guidance simulating the external provider communicating directly through the Tugboat avatar.

(4) The visual contents have been guided by explanatory categorizations unique in the system of the present disclosure as syntheses of biomedical, neuroanatomical, evolutionary, biopsychosocial, cognitive-behavioral and psychoanalytic models. The result is a unique synthesis between the ‘mental health visual analogical representations’ of mental health concepts and with the mental health ‘conceptual’ or ‘explanatory categorizations’ that can be presented digitally in the system of the present disclosure.

The approach taken in the MindSkiller eLEARNING service in the system of the present disclosure has been:

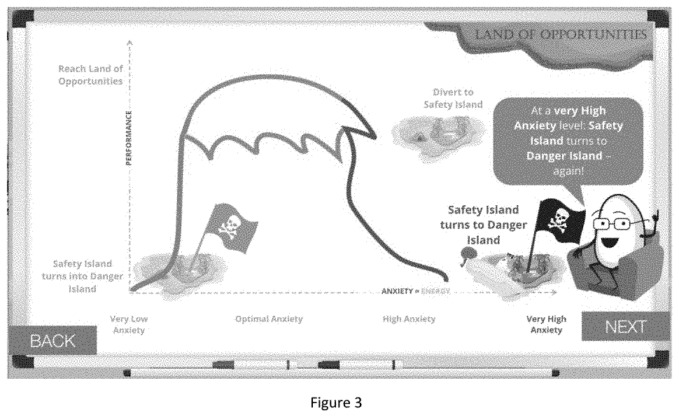

The approach taken in the MindSkiller eLEARNING service have been:
(1) DSM-5 based diagnostic categorizations and classifications have been included in MindSkiller eLEARNING—but an enhanced version has been developed that, in a somewhat disguised fashion, reflects an approximation of how psychiatrists utilize the DSM5 classification system in practice. It is a new categorization system that needed to be developed to both reflect the biopsychosocial formulation missing from DSM5 classifications but also retains a formulatory approach:
   Mind Subtype: due to an underdeveloped or immature capacity for decision-making and coping skills and unhelpful schemas, corresponding to the equivalent of ‘Psychological’ contributing factors from the biopsychosocial model
   Body Subtype: is due to ‘runaway feedback loops’ causing physical distress due to anxiety, pain, fatigue or nausea, triggered by social factors such as interpersonal stress, social disadvantage and distress corresponding to the equivalent of ‘Social’ contributing factors from the biopsychosocial model
   Brain Subtype: due to malfunctions, injuries and toxins, corresponding to the equivalent of ‘Biological’ contributing factors from the biopsychosocial model
(2) A course outline that has been divided into six Learning Units to reflect a highly novel curriculum syllabus to reflect a way of categorizing the study of mental health and its disorders that has not been approached in this way before:
   Unit 1: Mapping the journey to your life goals
   About getting your bearings—your starting and destination points, going in search of your life goals, learning how to reach them, and how to use coping strategies and recover from tipping into anxiety and slipping into depression along the way.
   Unit 2: Coping with external stressors
   Using your 3 coping strategies to stay healthy and function at peak performance—your rational, emotional and wise minds, how your rational mind functions under normal and extreme conditions, how your emotional mind functions under normal and extreme conditions.
   Unit 3: Coping with ups and downs
   Learn about coping with mood and anxiety disorders. For each of these categories, you will learn what it is (its descriptive features) and why it occurs (three types of causes—triggers, mechanisms and susceptibilities). You will learn how to beat it by using a Management Plan made up of Self-Help coping strategies, digital mental health tools and healthcare interventions from Help-Seeking (positive lifestyle factors, psychological therapies and physical treatments). There is a ‘Services and Information’ data matching service that links the user's specific information to the healthcare interventions of accredited providers and digital mental health tools and services.
   This Unit is divided into 3 Subunits:
   In SubUnit 3.1: Dr Egg and your Tugboat Assistant take you on A Brief Guide to Classifying Mood and Anxiety Disorders.
   Dr Egg shows you how there are 3 ‘angles’ to any disorder.
   First, each Banner of the eLearning Ship represents 1 of 6 possible categories of disorder. This is a descriptive classification based on core features. Here, Mood and Anxiety disorders is of interest.
   Secondly, Dr Egg shows how each Sail depicts one of 3 possible causative mechanisms—Mind, Body or Brain—which can explain how mental disorders develop.
   Finally, Dr Egg shows how each Stripe represents a specific Psychiatric Diagnosis. Each Mood and Anxiety stripe is a nickname based on how its symptoms combined to form its mental disorder.
   In Subunit 3.2: A Brief Tour of Mood Disorders
   Beginning with the Diagnostic Stripes opposite the Mood Banner, what they all have in common is learned—in other words, the core features of Mood Disorders.
   Then the groups of Mood Stripes allocated to each of the Mind, Body and Brain Sails is looked at. Each sail reflects the mechanism causing the Mood Disorders sewn onto its sail.
   Finally, it is learned how each single Mood Stripe is unique-based on the set of symptoms associated with it. After a brief examination of each of these Mood stripes, you are invited to conduct more in-depth tours of 3 of them—their features, causes and management plan.
   One stripe is hand-picked from each sail, allowing a comparison of the 3 mechanisms:
   PTSD-Acute stress from the Mind Sail
   Panic Disorder from the Body Sail
   Obsessive Compulsive Disorder from the Brain Sail
   In Subunit 3.3: A Brief Tour of Anxiety Disorders
   The core features of the Diagnostic Stripes opposite the Anxiety Banner is learned.
   Next, the groups of Anxiety Stripes allocated to each of the Mind, Body and Brain Sails is looked at.
   Finally, each Anxiety Stripe's symptom set is examined.
   After that, the same kind of tours as was done in the previous Subunit are conducted—here, of 3 Anxiety Disorders:
   Reactive depression from the Mind Sail
   Mixed anxiety-depression from the Body Sail
   Melancholic depression from the Brain Sail
   Unit 4: Coping with internal stressors: unhelpful belief systems and bad habits
   How you can be your own worst enemy from overreacting to events or harboring unhelpful self-sabotaging belief systems that work against your long-term interests.
   Learn about coping with personality disorders using the same approach used in the diagnosis subunit grid of Unit 3.
   Unit 5: Coping with being out of touch with reality
   Learn about what happens when reality is denied or distorted, in particular, about coping with psychotic disorders using the same approach used in the grid of Units 3 and 4.
   Unit 6: Coping with Fixations
   Learn about coping with substance use and eating disorders using the same approach used in the grid of Units 3, 4 and 5.
(3) Dividing our MindSkills (conscious mental faculties using free will) into 3 navigating skills (make decisions about engaging with life goals) and 3 coping skills (which help you deal or cope with external and internal stressors):
   (i) Navigation Tools
      Binoculars:
      Map:
      Steering Wheel:
   (ii) Coping Strategies
      Rational Mind:
      Emotional Mind:
      Wise Mind:
(4) Dividing all mental health concepts into triple categorization grids:
   (i) The 3 stages of mental health
      Recovery: Defined as “healing from mental disorders and becoming more capable at coordinating your own mental health care” and “beating a Diagnosis such as Mood, Anxiety and Substance Use Disorders. To do that, the negative effects of the lowered Mental Health—the symptoms, the loss of function and any interference with relationships and work need to be removed. The best-case scenario is discarding the Diagnostic Label.”
      Resilience: Defined as being “skillful at coping with stressful events and be less susceptible to developing a mental disorder. If they do have a disorder, have minimal disability and be less likely to relapse”
      Mental Fitness: Defined as being “capable of achieving and sustaining peak performance—the mental health equivalent of an elite athlete.”
   (ii) Dividing all Help-Providers into:
      Clinicians
      Mentors
      Coaches
   (iii) Life Goals divided into
      Garden of Activities: like studies and jobs, hobbies and exercise.
      House of Healthy Relationships: with your parents, friends, community and environment
      Valley of Values: your Guiding Values
         a. Attitudes of being assertive: being respectful, being calm & being diplomatic
         b. Being compassionate: open-minded, non-judgmental and accepting, warm and caring
         c. Being wise: self-reflective, self-empowering, skillful at choosing which coping strategy to use at different times
   (iv) 3 visual analogies making up the landscape during a mental health journey:
      Land of Opportunities: where you find your Life Goals
      Safety Island: represents a helpful defensive reaction. If the reaction is unhelpful, it is said to have turned into Danger Island.
      Red Ship: an event along the way—a threat (stressful event—depicted as Pirate Ship or ship with a black flag) or opportunity (depicted as the ship with a white flag)
   (v) 3 visual analogies representing the 3 routes or paths to the Life Goals
      Direct route to Land of Opportunities
      Direct route to Safety Island
      Roundabout route—via Safety Island then Land of Opportunities
   (vi) 3 components of your Performance Wave, an example is shown in FIG. 3: your ‘automatic reaction’ to events giving you 3 different types of energy (wave length) and performance achieved (wave height)
      Sensations: produce Motivation
      Emotions: produce Anxiety
      Thoughts: produce Willpower
   (vii) The 3 Brain Imbalances causing the 3 Clusters of Personality Disorders
      Imbalance 1 causing Cluster A
      Imbalance 2 causing Cluster B
      Imbalance 3 causing Cluster C
   (viii) 3 Navigation Tools
   (ix)-3 Coping Tools
   (x) 3 Empowering Questions to ask throughout a mental health journey to get the best health outcomes:
      a. What are my life goals?
      b. Hod do I navigate them?
      c. How do I cope with stressful events along the way?

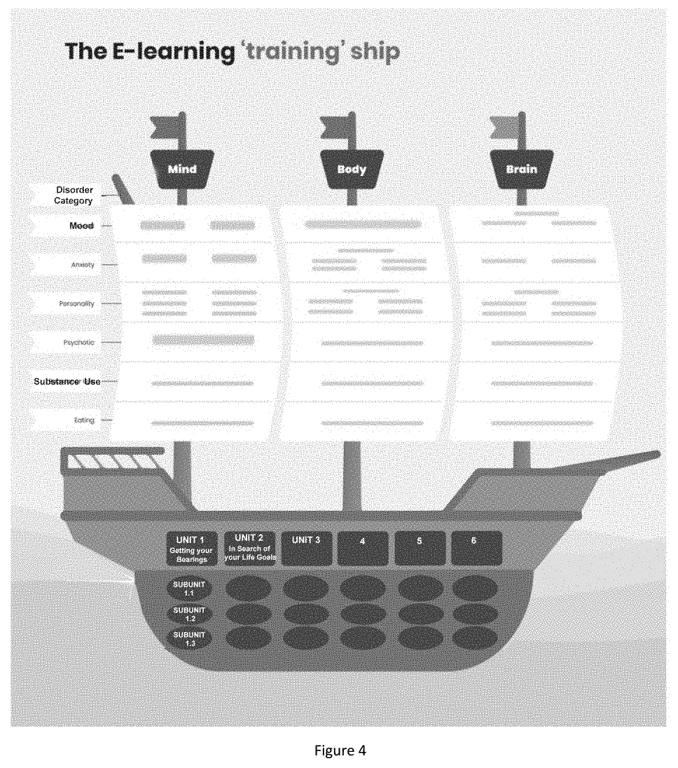

Reference is now made to FIG. 4 showing one of the eLEARNING Units. In an embodiment of the present disclosure, the Biopsychosocial model is modified such that the approximate equivalency mapping is:
Bio→Brain Mechanism
Psycho→Mind Mechanism
Social→Body Mechanism

There is no other digital mental health services has come close to the present disclosure incorporating all these:
Models of care and treatment
Principles of care
Healthcare values and ethos

The Learning Units incorporate all the current models of care and treatment so it is multi-modal: person-centered care, trauma-informed care, multidisciplinary care, shared care, blended care, stepped care and staged care, whilst protecting users' confidentiality based on healthcare values.

The Learning Units are based on autonomous learning principles to make the education and training service highly engaging, practical and sophisticated.

The Learning Units use true visual learning methods creating a theme-based virtual universe of seamlessly integrated visual analogies that removes technical jargon and makes the concepts understandable to any user be they a patient with no prior knowledge or training, to mentors, coaches and clinicians both medical and non-medical.

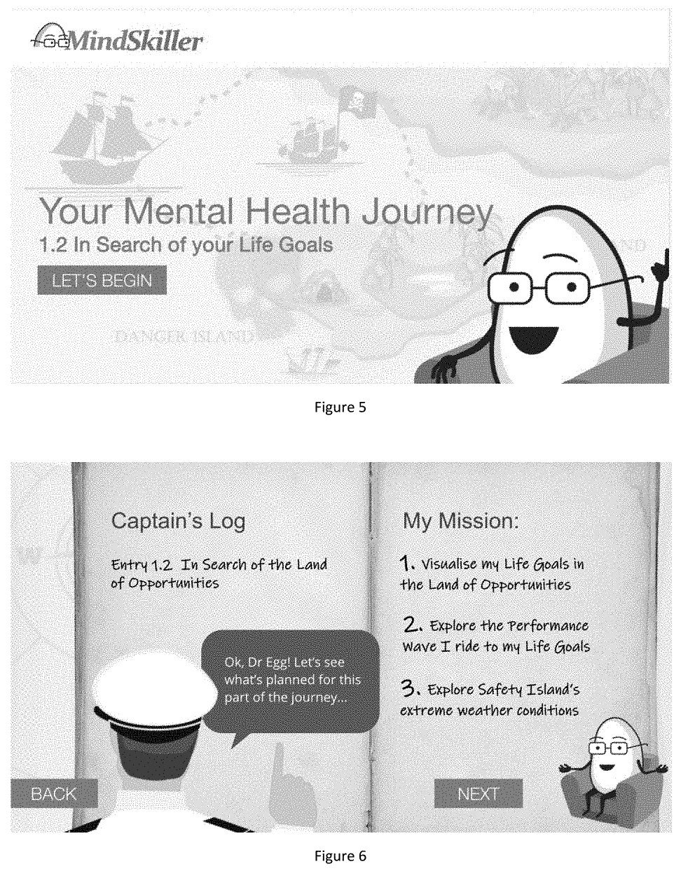

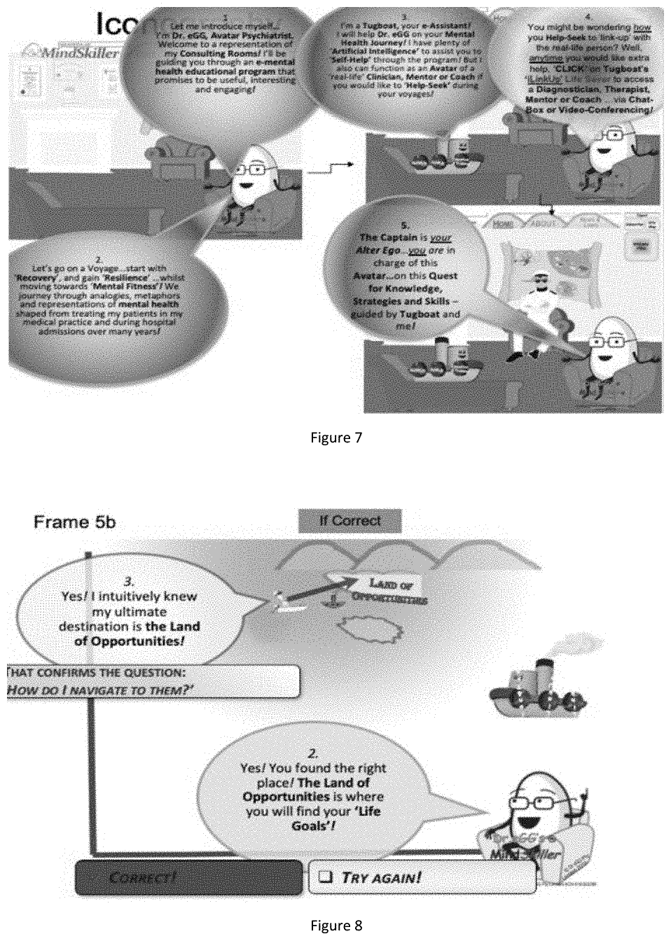

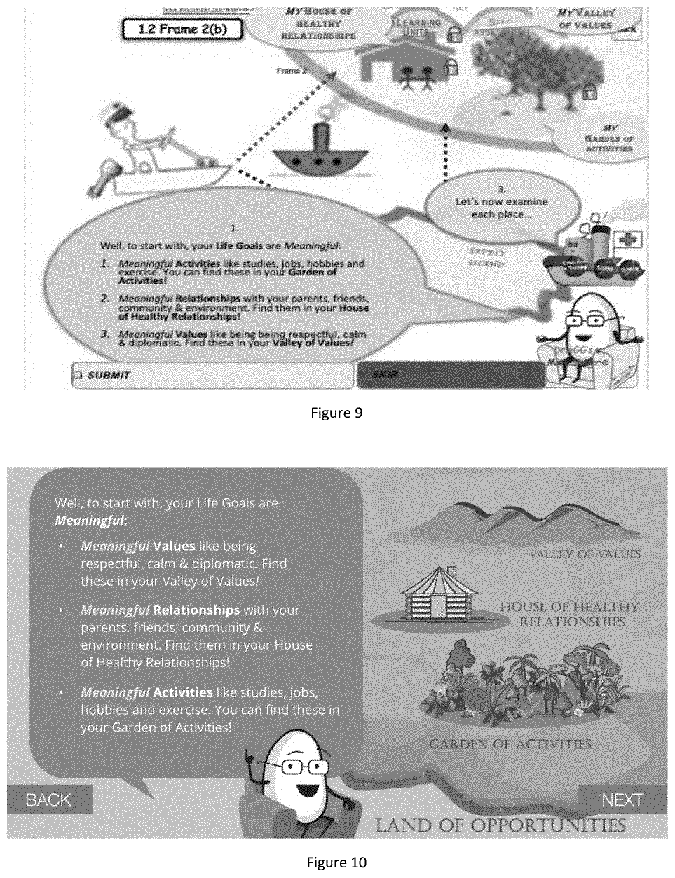

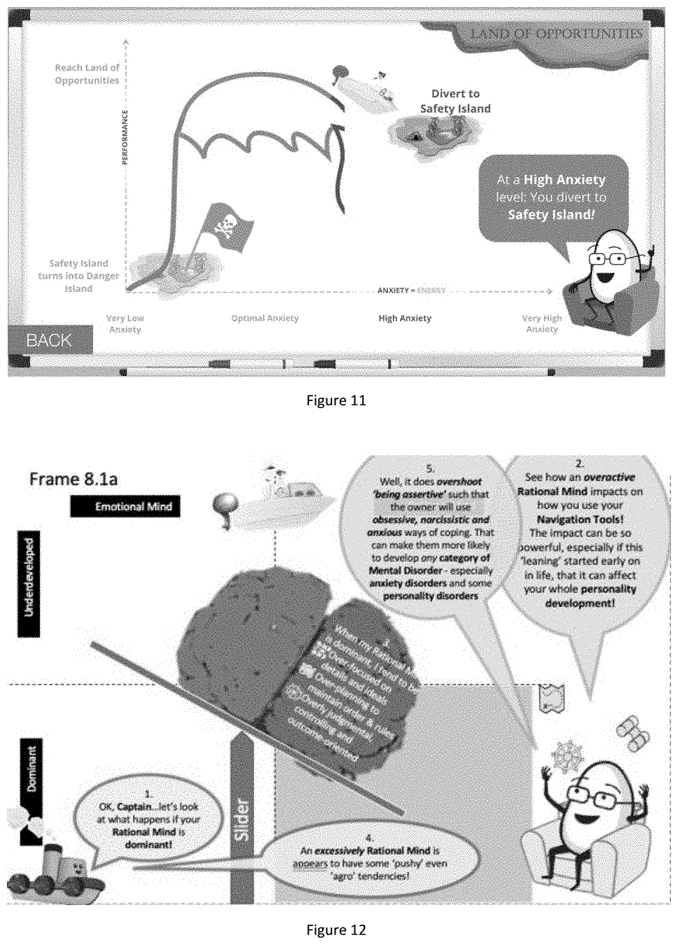

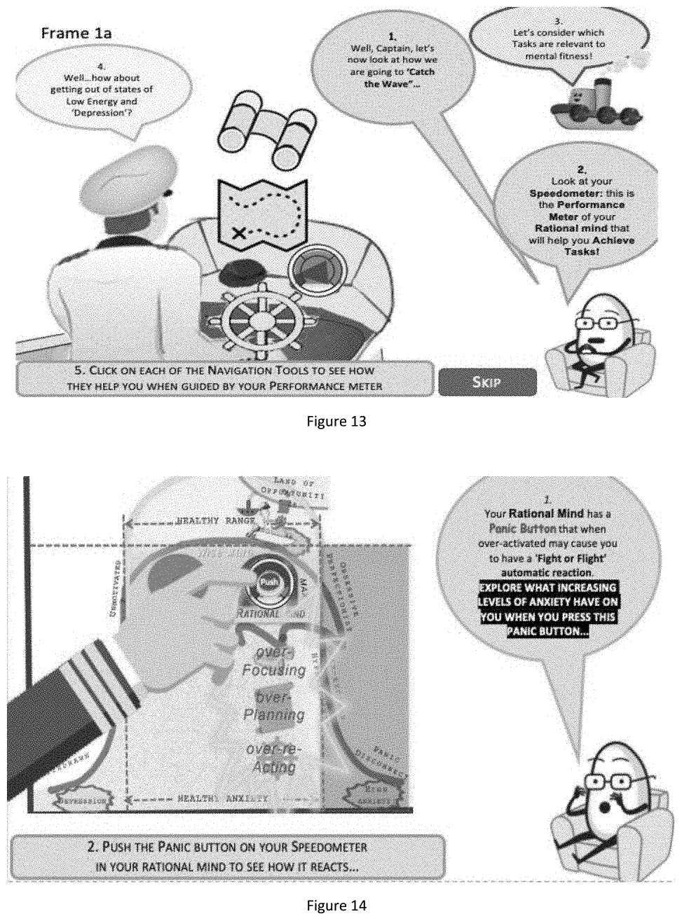

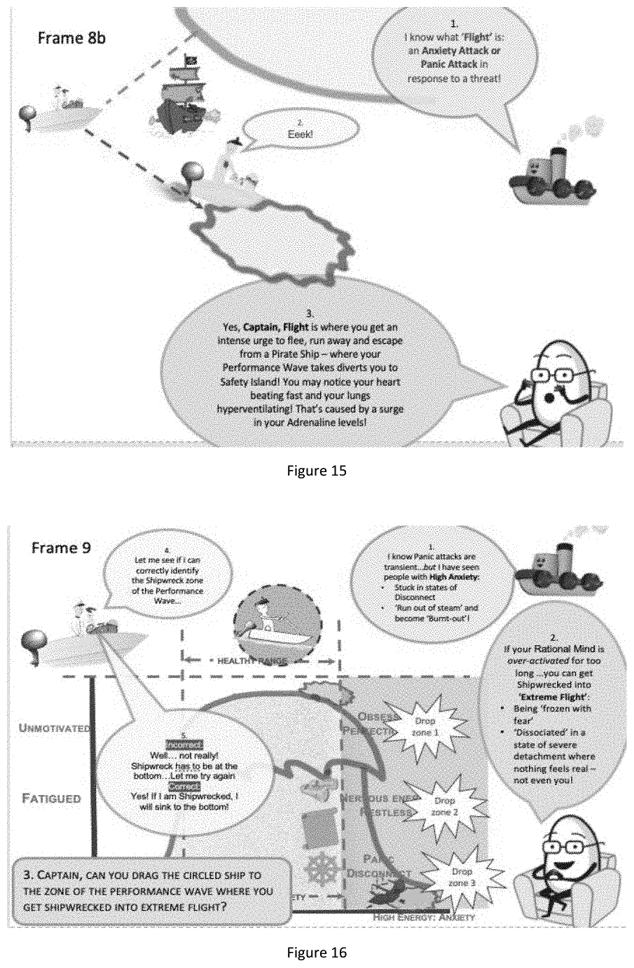

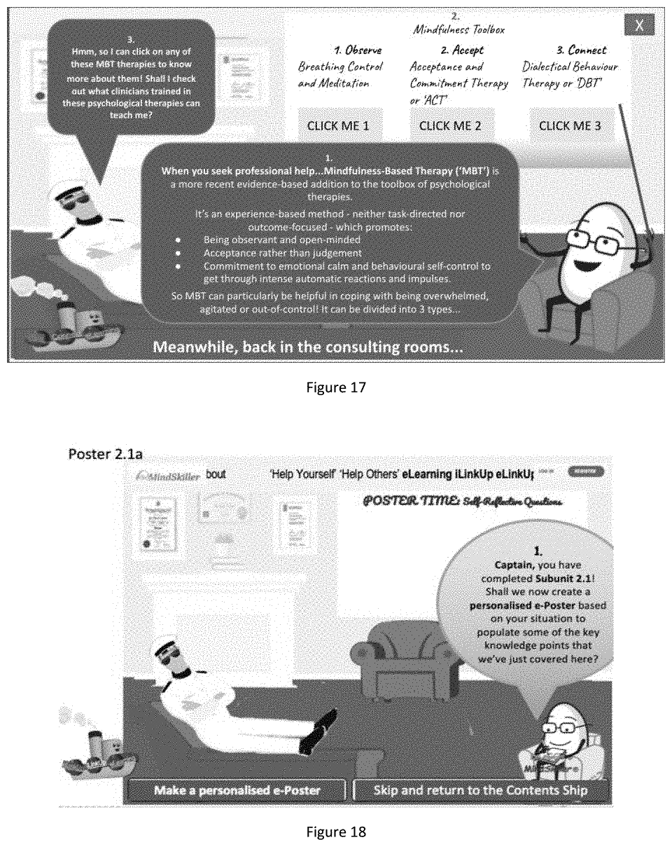

FIGS. 5 to 18 shows the narrative of the Learning Units of the e-Learning Ship being a preferred embodiment of the present disclosure.

An operationalized stepped care ‘clinical support’ service enabling users to access immediate real-time brief episodes of Telehealth support and guidance from accredited providers whilst the users complete the Learning Units. Here are some key attributes:

(i) Broad access to “mentors” (e.g., peer supports, family members, case managers) and “coaches” (e.g., exercise physiologists, certified counsellors, certified coaches e.g., ADHD coaches) as well as “clinicians” (general practitioners and specialist medical practitioners, psychologists, mental health nurses, occupational therapists, social workers and other ‘clinical’ health providers) will be built on top of and integrated into the eLEARNING platform, to create a true virtual hospital and community mental health infrastructure.

(ii) Embedded clinical care pathways providing help-seekers and help-providers a structure to interact together

(iii) Healthcare-styled accreditation and quality control measures and processes paralleling real psychiatric hospitals and mental community mental health services, together will optimize quality control

(iv) Designed to supplement and complement primary care, specialist practice as well as cater to non-referred consumers seeking high quality digital mental health self-help services actually developed completely by a psychiatrist rather than the narrow scope of other digital mental health services available.

(v) It will enable users to selectively give their existing health providers permission to monitor their progress.

(vi) Providers will be required to undertake the eLEARNING program to gain accreditation. Higher levels of accreditation will require providers to themselves undertake specified Telehealth support. The program may also serve as a source of CPD for their own regulatory bodies or associations.

Expandability to provide users and providers with access to other digital mental health services that are innovative, translational and complementary to the MINDSKILLER® platform, content, vision and healthcare values.

The functionality and features required for the Minimum Viable Product (MVP) of an embodiment of the present disclosure is described below.

The MVP, subsequently referred to as Version 1.0 or V1.0, is a mental health recovery-oriented pilot version of the MindSkiller eLEARNING Education and Training e-mental health program that has been developed as a prototype with a vision to be able to be utilized by various types of users:

(i) Consumers self-identifying as wanting ‘self-help’ and/or to ‘help-seek’ to recover from a mental health condition

(ii) Consumers who have been identified by a treating clinician as likely to benefit from utilizing an online ‘self-help’ program and/or ‘help-seek’ to recover from a mental health condition

(iii) Consumers who wish to serve as ‘Help Providers’ in the form of ‘Mentors’ to consumers who are seeking help, which may include:
Family, carers and friends
People who themselves have had a so-called ‘lived experience’ of suffering from or having recovered from a mental health condition
Case Managers from healthcare or para-healthcare organizations such income protection health insurers, workers compensation insurers, health insurance rehabilitation programs etc.

‘Help Providers’ who are existing local health board-certified ‘Clinicians’ who wish to assist and guide help-seeking consumers to improve their mental health literacy
There are two approaches clinicians are likely to adopt:
   using MindSkiller as additional help for their existing or past patients in complement to their current or past face-to-face or online care
   assisting new patients
This group includes:
   Psychiatrists—medical specialists who have the highest mental health skillset and can provide diagnostic clarification and guidance, formulation with prioritization and diagnostic hierarchy, knowledge of all the modes of treatment and interventions, familiar with all mental health care models and principals, and assist with management package options
   General practitioners—noting that some have advanced mental health training
   Allied health clinicians such as Mental Health Nurses, Psychologists, social workers, Occupational Therapists and Speech Pathologists

Coaches include non-clinicians supporting people recovering from mental disorders such as
Personal trainers
Life Coaches
Counsellors and other certificated coaches
Other allied health providers who would not identify as being a clinician such as Pharmacists.

In version 1.0, there is provided three Learning Units that will be fit for these 3 priority purposes:
Beta-testing a cohort of ‘self-help’ users
Recruiting some of the ‘self-help’ beta-testers to serve as ‘help-providers’ for the next stage of beta-testing, which would be to assess the program's capacity to serve as a tool to enable ‘peer supports’ to assist ‘help-seeking’ users (this is one of the types of ‘mentors’ who can become accredited to use the next version of the program)
Serving as a demonstration version of the full prototype's and business plan's potential vision for the purpose of capital-raising for the next stage of development post-pilot.

This document is supported by a sitemap and content deck.

Several workshops were run on the lean canvass, user journey—both for ‘Self-Help’ users and ‘Help-Provider’ users (Clinicians, Coaches and Mentors—which includes peer supports). This has now been translated into this functional specification.

The online journey of the eLEARNING Units is hereby described.

When users come onto mindskiller.com they can browse the site freely, without initially registering. It is important that there are no barriers to exploring the top-level ‘marketing and information’ website and getting a feel for the content and method.

The homepage will explain what MindSkiller is all about. It will have a short video by Dr Egg that explains this, rather than extensive copy.

The user will also be able to see the overall site navigation, with the homepage providing a quick overall summary of the complete site.

The key sections of the website are:
Home
About
Help yourself (Self-Help)
Help others (Help-Providers)
The 3 digital mental health services MindSkiller is providing: eLEARNING, CONNECT and PLUS
Login/Register/MyAccount

The user will then be funneled to identify whether they are looking to:
‘Self-Help,’ which means help themselves using the education and training content without any online ‘clinical support’ (CONNECT video conferencing or chat box with an external clinician, mentor or coach) or
‘Help-Seek’—‘Self-Help’ plus online ‘clinical support’ (CONNECT video conferencing or chat box with an external clinician, mentor or coach) or
‘Help-Provide,’ which means helping others (providing online ‘clinical support’ using CONNECT video conferencing or chat box with an external clinician, mentor or coach)

This is the basic segmentation of the demand and supply sides of the mindskiller.com platform. This ‘tick box’ is so they can be directed to the patient (Self-Help′ and ‘Help-Seek’) content or the ‘Help-Provider’ content for the clinicians, mentors and coaches who want to become accredited to help others using MindSkiller CONNECT.

Note that another embodiment will need to differentiate two types of ‘Help-Provider’ when they click that option:
Individual providers wanting to provide individual mentoring, clinician care (diagnostics, treatment, therapy) or coaching assistance using CONNECT
e-mental health service providers like virtual reality therapies, game-based interventions, future physical therapies that can be connected online enabling control by clinicians remotely e.g., Transcranial magnetic stimulation (TMS), and other innovative interventions that use digital information and communication technologies to improve mental health care) using PLUS

Brokering Help-Provider organizational services as well as individual/sole help-providers

A huge gap is the lack of ability for consumers and providers to access innovative new interventions that are built in silos with huge potential that never get off the ground due to lack of knowledge about them and take-up. Large companies have not seen the potential in developing medical devices for mental disorders and despite promising research they simply don't seem to attract large funding like physical conditions do.

In one embodiment, the system disclosure provides the PLUS services for linking to an external provider's webpage that meets the requirements to integrate with the software. This service provided by the system of the present disclosure could facilitate innovation with TMS, there is no reason a TMS machine (or other reasonably safe electronic or electromagnetic stimulation medical device that does not need a clinician operating it where the patient and device is) couldn't be purchased by a consumer who keeps it at home and connects it via software to a psychiatrist's practice who sets the dose, monitors the response and hence treats the patient remotely. There are some extra procedures related to its use that need to be done before actual treatment can start (remotely), but even these could be done remotely. There are caps (look like a blue surgeon's head cap) that fit over the patient's head that help to determine the location for the TMS paddle. That procedure is a visual one, which the psychiatrist could do remotely using video conferencing. Currently, there are concerns about how to administer TMS safely due to the pandemic. The current system is design to control and connect the TMS remotely.

The system of the present disclosure integrates virtual reality with the TMS. The information/communication technologies and the treatment methodologies are both available. What's missing in the current art appears to be creative, visionary entrepreneurs. The potential for developing innovative e-mental health services has never been better than right now. It is anticipated that innovative service providers will want to utilize PLUS to access consumers and providers.

For one embodiment of the present disclosure, the user will just be asked to select one option only, so forcing one decision:
Self-Help
‘Help Self and Others’ (Mentor Help-Providers)
‘Help Others’ will need to differentiate individual Help-Providers (Clinicians and Coaches who will use CONNECT when they are accredited) and a company or team mental health service (who will be accessible via PLUS).

In one embodiment of the present disclosure does not ‘direct’ users what to do, rather it need to be seen to ‘guide’ users to learn Self-Help by providing them with educational material and to ‘match’ Help-Seekers with a spectrum Help-Providers to improve their mental health literacy and capacity for ‘self-coordination.’

A link to a full privacy policy and legal disclaimer will be available.

The home page will also have some summary information of the other tabs and sections, with links for more detailed content.

Patient journey (“the lone ranger” or ‘Self-Helper’) of an embodiment of the present disclosure is described below.

Once a patient has identified themselves, they will be asked to select one of five “Areas of Mental Health Interest” and “Mental Health Disorders” (6 categories of mental disorder) from the interface of the system of the present disclosure. This is to determine if they are just browsing/interested, aware of their disorder or have been previously diagnosed.

This is not compulsory, but MindSkiller will explain that this will help direct the user to the right information. When they register, they will be asked more about the category of disorder they are interested/suffering with and what area of interest they have—which will equate to the Learning Units.

The user will also be given the option of non-disclosure or don't know. In one embodiment, they are given the opportunity if they tick this ‘don't know’ box of arranging a PLUS diagnostic mental health service. For the MVP of another embodiment, it is shaded out for the purpose of demonstration.

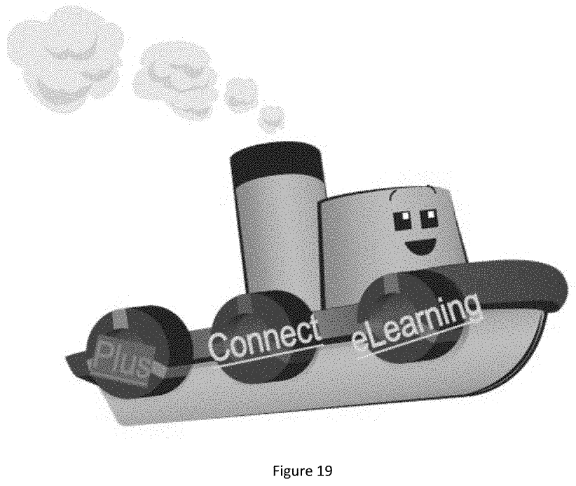

In one embodiment, the Tugboat as shown in FIG. 19 represents an electronic Assistant that can give guidance. It represents a clinician, mentor or coach. At any stage, the user can click on the Tugboat's Life Preservers to connect to a:
Clinician Diagnostician
Clinician Therapist
Mentor (Peer support)
Coach

The Tugboat needs to be present on most of the screens of the interface to give this option via its CONNECT life preserver image hyperlink. With a limited version of CONNECT active in the MVP, it is possible to explore whether the life preserver image hyperlink can be active as well as visible on the Tugboat to provide its add-on services, in addition to the usage of the Intercom plug-in software that will serve as a partial CONNECT service.

There should be consistent visuals from the front-end interfaces through to the e-Learning content.

Each mental disorder (in the MVP, anxiety disorders and mood disorders are prioritized) will have a high-level overview, presented using the analogy of a journey at sea, with some key characters and avatars, including a Captain—representing an ‘alter ego’ of the patient, the Captain's ship accessories (representing the tools to navigate the boat and cope with stressors—which are the two sets of 3 mental faculties that will be upskilled or matured by undertaking the program) and engine (representing the brain's automatic responses to events—involuntary mental inner subjective experiences), a Tugboat (representing any of the various help-providers—this gets the user used to having a ‘team’ approach to their care in case they do need to seek external assistance to manage their mental health or to recover or to achieve their mental health goals) and other graphics to explain mental health disorders in layman terms.

If the user then wants to learn more, the site will direct them to an online learning platform where they can sign up for some e-Learning. All users have to register for access to all subunits in Learning Units 1, 2 and 3, which in the MVP are all free. In return for this free learning material, they need to exchange this personal data (In other embodiments, these Learning Units will remain available whilst there will be a charge for access to further Learning Units).

The Registration and login interface of an embodiment of the present disclosure is now described in more details.

The user will be asked for their name, gender, date of birth and email address. The email address will serve as their username. The registration page will identify whether they come through from Self-Help or Help-Provider and populate any data that has already been captured e.g., area of interest, disorder selection.

When the users register, they will receive an email to authenticate their identity, including a process to ensure they are not a bot. In one embodiment, the authenticate process would probably require two stage authentication.

Once they have clicked on this email, they can set a password. Registration will allow them to automatically log back into the website and learning management software (LMS).

The eLEARNING services of an embodiment of the present disclosure is now described in more details.

Once they are registered, the user can access the e-Learning units.

The user can undertake the journey in a number of ways:

(1) Do the Subunits sequentially including all quizzes, completing all the content and all the quizzes they then pass to get the Certificate 1 in one preferred embodiment;

(2) Do everything sequentially except completing all the quizzes in which case they don't get a certificate. However, the completed content and whatever they've completed of the quizzes is stored in a database, so that they can come back anytime to complete all required content and quizzes to get the Certificate;

(3) Look through any Learning Unit content and quizzes in a non-sequential self-directed way. Whatever is completed is stored. They can come back to complete the gaps. That includes the option of going directly to explore specific Mental Disorders.

In one embodiment, Version 1 of the eLEARNING system has designed to include 26 individual training modules across Units 1-3 including:
1. TRANSDIAGNOSTIC UNITS/SUBUNITS: mental health ‘Areas of Interest’ (10)
2. KNOWLEDGE CHECK SUBUNITS: the associated ‘knowledge subunits’ or quizzes (10)
3. DIAGNOSIS SUBUNITS: disorder-specific all with the same template (Features, Causes, Management Plan, Posters, Links) (6)

In another preferred embodiment, Version 2 of the eLEARNING system will include an extra 27 individual training modules across Units 4-6 including:
1. TRANSDIAGNOSTIC UNITS/SUBUNITS: mental health ‘Areas of Interest’ subunits' (9)
2. KNOWLEDGE CHECK SUBUNITS: the associated ‘knowledge subunits’ or quizzes (6)
3. DIAGNOSIS SUBUNITS: disorder-specific all with the same template (Features, Causes, Management Plan, Posters, Links) (12).

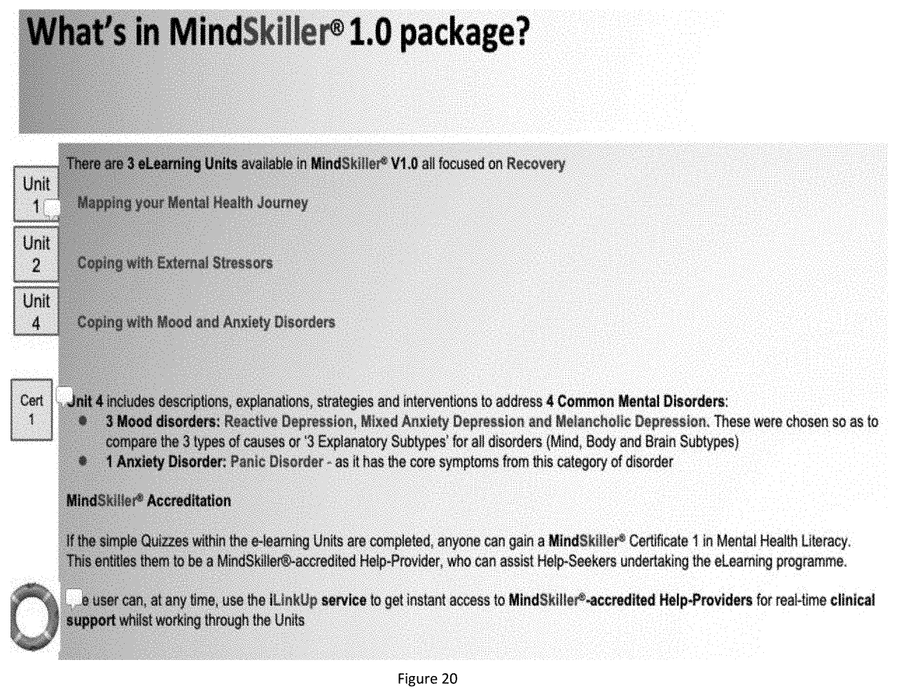

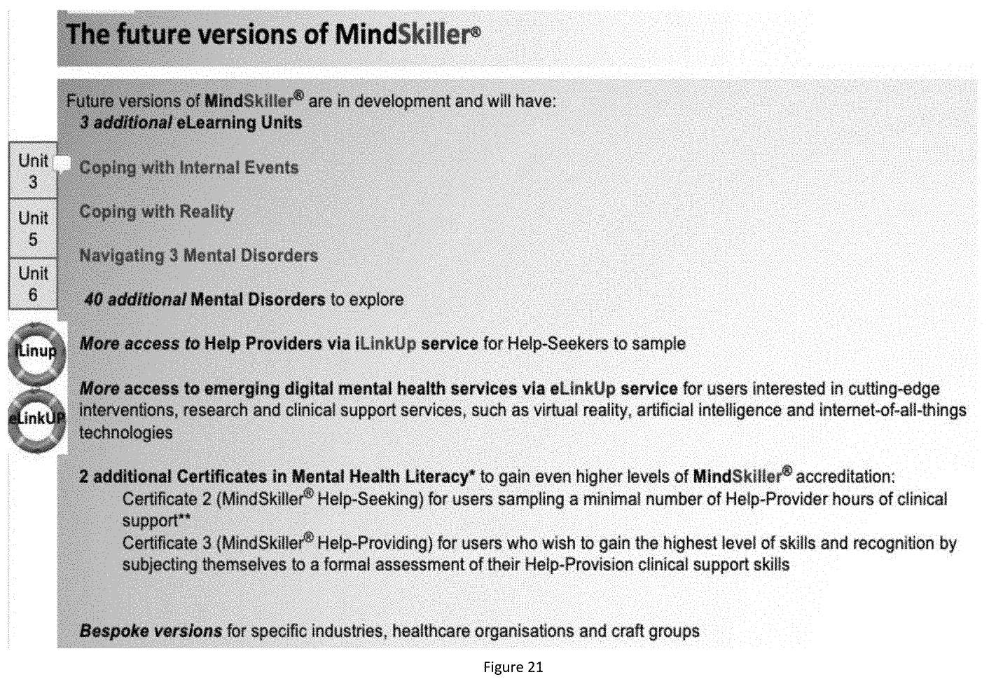

Reference is now made to FIGS. 20 to 21 showing system of an embodiment of the present disclosure. The system comprises an eLEARNING system having:
6 Units, each of which are made up of 3 Subunits;
a more sophisticated, personalized and tailored recommendation of which Units/Subunits/Disorders for the user to prioritize and focus on, based on the Areas of Interest and Mental Disorders that they identified. There will be a simple set of rules to determine the recommended Units/Subunits/Disorders based on their selections. Ultimately, an advanced AI algorithm will need to be utilized to identify the best approach (profile and order of Units/Subunits/Disorders) for the individual user. The recommended profile and order of the user journey for the individual user will be provided by the Dr Egg avatar. That tailored user journey could be modified by the user in collaboration with their authorized external clinician and accredited clinicians that the user accesses during their journey;
The user will be asked which specific mental disorder they would like to explore from the 40-45 mental disorders available. This information is then appended to their ID and revealed in their Dashboard (accessible via MyAccount, where any disorder categories indicated to be of interest from the ‘marketing and information’ website are listed); and
the Units/Subunits/Disorders be presented in a summary form (or highlighted in the ‘contents’ page) so that the user can click on them to access the specific education and training content.

In one embodiment, the eLEARNING system may provide a short video introduction for each Unit/Subunits/Disorder. At the beginning of each Subunit, there may be a Captain's Log that summarizes the key points to be addressed in the Subunit. At the end of each Subunit, the Captain's Log will summarize the key points that were addressed in the Subunit.

Either at the end of a Subunit (Plan A) or embedded within the content of the Subunit (latest plan—Plan B; probably will be compared doing both for beta-testing) will be 3 quizzes:

(1) Multiple Choice Questions (probably not with plan B)

(2) Line-matching Questions and Answers

(3) Self-Reflective Questions (these will remain at the end of the Subunit even in Plan B).

The text answer to the self-reflective question is retrieved from the system database and inserted into a personalized ‘management plan’ interface, which can be converted into different format, such as printed, downloaded or emailed as a PDF.

Once the user has completed the training they are issued with an official personalized Certificate 1 that can be printed, downloaded or emailed as a PDF. This will enable Certificate 1 MindSkiller accreditation to be a Help-Provider using the CONNECT software features.

Training/Accreditation Certificates: Current plan comprises:

1) Certificate 1 (also referred to as “Cert 1”) be provided after the user completes Units 1 and 2 in one embodiment.

2) Certificate 2 (also referred to as “Cert 2”) be provided after the user completes Unit 3 in one embodiment.

3) Certificate 3 (also referred to as “Cert 3”) be provided after the user completes Units 4, 5 and 6 in one embodiment.

Because this is a virtual health service of the present disclosure is adapted to accrediting users, there system will be configured to ensure a reasonable quality control. If it combines the quality of control of the training with the actual eLEARNING program itself, that's a novel way to provide a ‘virtual hospital/community mental health service.’ It is similar to how hospitals work in that the structure of the hospital's wards, admission and discharge processes, accreditation of clinical staff, and clinical programs all working together provides a quality healthcare service. That is what's missing from most online digital mental health services—a sophisticated underlying ‘clinical care pathway’ so that is what is being developed to fill the gap. A virtual clinical care pathway to both enhance recovery, the development of resilience and mental fitness that is truly complementary and supplementary to existing face-to-face care pathways and improves efficiency and the user experience of their mental health journey. There are attributes that make it superior to any other digital mental health service—it incorporates all the models that are considered modern, cutting-edge and ethical in psychiatric mental health care—especially being that it truly puts the user in the center of the system.

One embodiment is to require providers to undertake ‘basic training’ using the eLEARNING software (e.g., Cert 1 or Cert 2), to enhance the mental health care of external unaccredited help-providers, including features such as:

1) feedback provided to the health provider in the form of ‘Progress Reports,’ which are user-controlled. This feedback can include some basic information that will itself educate the external provider and improve their mental health literacy in doing so;

2) regular MindSkiller newsletters provided to keep the health provider up-to-date with services and products that can enhance their help-provision of their patients and clients with mental health problems;

3) inviting the external provider to undertake the training to obtain MindSkiller-accreditation to use CONNECT to support their patients/clients/mentees by completing eLEARNING Certificate 1 or 2 and to acquire CPD points;

4) providing access to the disorder-specific Diagnosis Subunits (Stripes on the Sails of the eLearning Ship), which general practitioners, psychologists and other providers can use to look through with their patients.

In one embodiment, the system is designed to be developed based on professional experience and revising hospital by-laws and the system is adapted to provide:

(1) a module engagement for pulled data through to the users' profile and dashboard interface within a myAccount interface;

(2) a user administrator adapted to reset passwords, amend data etc. There be interfaces for access to specific database information (e.g., Dashboard and Self-Assessment Response data) by:
User-approved external clinicians;
Accredited clinicians, mentors and coaches; and
Top-layer Executive-approved Researchers (e.g., Dashboard and Self-Assessment Response data).

(3) disorder links interface that directs the user to a Links page with pointers to further information on:
medications and other physical treatments
psychological interventions
positive lifestyle choices

There will be ‘shortcuts’ to CONNECT Help-Providers who have listed a matched specific intervention with the user's intervention of interest, which they are skilled in and willing to provide assistance with.

(4) a dashboard interface within the myAccount interface where the basic information that is being collected by the system of the present disclosure through a website interface is held in a user profile (MyAccount) and can be accessed at any time through a user's nominated password. This Dashboard will also allow users to self-edit and change their details, change their password etc. The Dashboard will be adapted to display a record of the training they have been recommended and the training they have done. The Dashboard will capture the following key analytics:
Time spent by user navigating Unit/Subunit/Disorder
Completion of Units/Subunits/Disorders
Completion of specific quiz questions
Completion of quizzes
Access to posters (prints, downloads, emails, shares)
Use of any hyperlinks in ‘Links page’ to access additional information and services
Completion of Self-Assessments (highest privacy controls needed)
Time spent by user purchasing any Mentor/Clinician/Coach help-provision via CONNECT and full detail record (date, name and profile of help-providers, access shortcut via CONNECT)
Time spent by user purchasing any innovative service-provision via PLUS and full detail record (date, name and profile of digital service providers, access shortcut via PLUS)
Data access record by user, accredited help-providers, external clinicians, researchers, company staff etc.

(5) Help-Provider journey interface for Clinicians, Mentors and Coaches. In this system, the Help-Providers can be an object class defined as Mentor, Clinicians, and Coaches.

Mentors may include:
Peer supports (existing or previous patients)
Family, friends and carers. This includes community-orientated people such as senior students, recent school leavers, university students, advocates and lobbyists
Case managers working in a private health insurer, worker's compensation insurer, disability employment service, community mental health service, non-government organization health or psychosocial service provider.

Clinicians can be Diagnosticians or Therapists, wherein Diagnosticians may be Psychiatrists, General Practitioners with advanced mental health training/mental health General Practitioners, or Pediatricians; and Therapists may be Psychiatrists (prescribing medication is pharmaco-therapy), Psychologists (CBT, mindfulness and narrative or insight-oriented therapies), or Allied Health clinicians, such as Mental health and general nurses, Social workers, Occupational therapists or Speech pathologists.

Coaches can be Counsellors, Personal trainers, Life coaches or alternative therapists.

After the user has identified themselves as interested in being a ‘Help-Provider,’ they will be asked to Register themselves with MindSkiller of the embodiment of the present disclosure.

In one embodiment the MindSkiller system will carry out one or more of the following steps:
(1) User asked to Register for the Help-Provider Beta Directory. They will need to provide details of their professional credentials, category of clinician, professional qualifications, their CDP provider, indemnity insurance cover, etc.
(2) Once the users have registered, they will be diverted to a landing page where it will be explained to them:
   about the Help-Providers' Directory and the benefits of being listed in it—that, once they have completed training and accreditation, they can be selected by the patient for a Telehealth consultation based on their areas of interest and expertise (treatment modalities, disorder expertise or experience, areas of interest)
   what MindSkiller will do with the information about them
   how to complete the training.
(3) The user will be encouraged to undertake the Learning Units that will enable them to be accredited as a MindSkiller-accredited Help-Provider. The motivation will be that they will be:
   prioritized in the patient directory
   across the same information that the patient has been through.
(4) The training database needs to ‘rate’ the help-provider in the directory based on their training level completed. In one embodiment, the system of the present disclosure is divided to those that are MindSkiller-accredited and those that are not. In another embodiment, there is provided algorithm-based MindSkiller-accreditation, qualifications, relevant disorder experience etc.

This system is designed to encourage Help-Providers to registering who are not ready to do the MindSkiller training. This is a good prospect pool to engage and communicate with and persuade over time to trial and complete the training.

More detailed information is required from the Help-Providers to make sure they are qualified to provide “mental health guidance” for the purposes of optimizing mental health literacy outcomes of users whilst undertaking the educational program. This information can be input to the system through a registration interface.

The information required by the system may include evidence of qualifications, such as:
AHPRA Registration
Other accreditation bodies (e.g., overseas healthcare practitioner boards)
Membership of Colleges, Societies, CPD organizations
Number of years' experience
Evidence of training programs and/or courses in specific therapeutic modalities:
   a. CBT (cognitive behavior-based therapies)
   b. Mindfulness-based therapies e.g., DBT, ACT
   c. Insight-oriented/psychodynamic/schema/narrative-based psychotherapies
   d. Specific pharmacotherapies relevant to the management of mental disorders.

The system of the present disclosure is adapted to provide ongoing engagement by various method such as published Newsletter to each user device or providing a blog or news feed for user device to fetch latest information. In one embodiment, all users registered will receive an email update in the form of a Newsletter (one for Self-Help user, one for Help-Providers). In another embodiment, the system may maintain a website Blog section to include information to encourage users back to mindskiller.com.

In one embodiment, the present disclosure is implemented using a Software as a Service (“SaaS”) cloud-based site for the marketing and information site (October CMS), and an open-sourced platform for the LMS (e.g., Moodle).

It is preferable that the Database 74 and Authentication system 64 as shown in FIG. 1 is implemented in separate hardware, so that MindSkiller can be re-platformed easily.

A basic campaign management platform will be required to automate the update emails and track engagement.

Both sites need to be able to test and learn different content and capture feedback from the users. The test site needs to provide detailed analytics and reporting on user behavior on the site.

It is preferable that face-to-face interviews may be conducted with the user to get qualitative feedback on the site or conduct remotely through digital conferencing system.

In another embodiment, security needs to be high to protect the privacy of the sensitive personal medical information provided. The system of the present disclosure may have different options to optimally protect the user's data and protect from cyber-attacks. In one embodiment, two-stage authentication is implemented as a standard process:

In one preferred embodiment, there is provided 3 digital mental health service sections. The 3 digital mental health services are represented by life preservers on Tugboat as shown in FIG. 19. The tugboat has three life preservers for eLEARNING, CONNECT, and PLUS. These graphic needs to be updated so that the life preserver rings are visible and labelled. The eLEARNING service is represented by a Life Preserver colored blue. The PLUS access point is represented by a Life preserver colored green. The CONNECT access point is represented by a Life preserver colored red.

In one embodiment, there is provided a landing page for the 6 Learning Units and 18 Subunits, using a ship analogy—the ‘eLearning Ship.’ In addition to each Learning Unit and Subunit, the website needs to give an overview of all this eLEARNING program so that the user can understand the total program and its structure, like the syllabus of a course. The eLearning ship shows the interrelationship between the eLEARNING transdiagnostic, knowledge check and diagnosis subunits, including:
1. Full page eLearning Ship
2. Cut down ship that can be an “icon” on key pages that you can click on to come back to the full-size eLearning Ship for more detail. Ideally the location of the eLEARNING unity lights up on the icon.
3. Specific graphics for details of the ships and to be used in the eLEARNING navigation page (red)
   a. Large rectangular porthole aperture (to signify the Unit)—six in a row on the hull of the ship. These are big enough to be labelled.
   b. Smaller circular porthole apertures under large rectangular aperture to signify a Subunit, three under each Unit.
   c. Crows' Nest on each mast above the sails with a label of Mind, Body and Brain
   d. Three Sails, with six sections and ‘stripes’ to represent the disorders.
   e. Stripes to represent the specific disorders with links to their Diagnosis Subunits categorized using a grid of disorder categories (Banners) on the x-axis and Sails on the y-axis (Mind, Body and Brain).

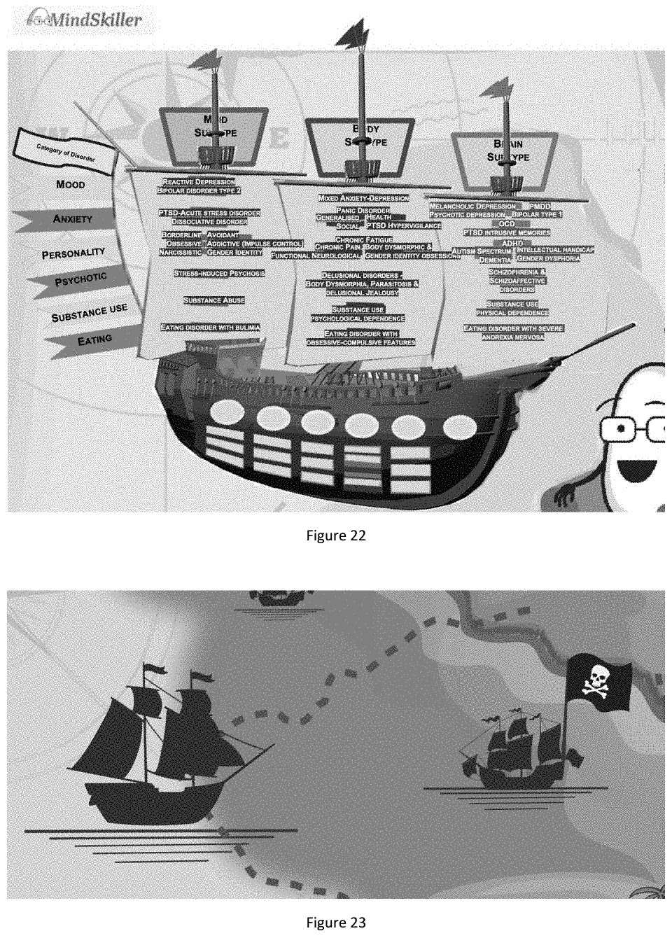

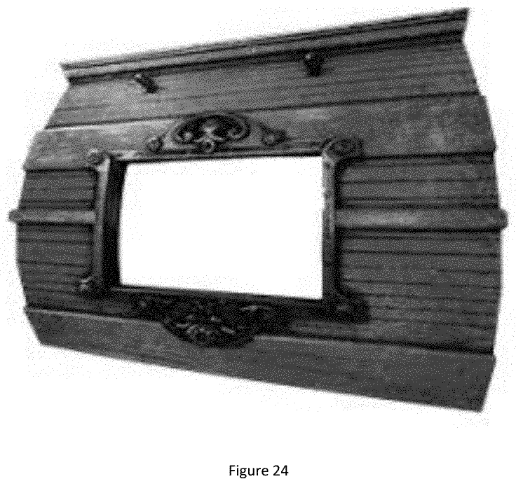

In one embodiment, the ship design as shown in FIGS. 22 to 24 comprises the following concepts:
Ship specifically designed to incorporate the key visual analogies but inspired by a Man of War or square rigger (see ref below)
Some consistency with some of the ship graphics in the Learning Units (See map ref below)

The full-size eLearning Ship in an embodiment of the eLEARNING service of the present disclosure with detailing is now described. Rear flags or banners of the Ship are designed to fly horizontally so they can be labelled with the 6 disorder categories. One option is to have a single flag with the title “Disorder” so each flag only needs one word in each flag. Sails are configured to have 6 sections, which line up with these disorder categories. For example, the Mood Banner above the red line on the graphic below needs to line up with the relevant section or square of the Mind, Body and Brain sail. Preferably, the sail is divided into 6 sections, rather than 6 separate smaller sails. There are blue, red and green colored “stripes” that signify the number of diagnoses or Diagnosis Subunits in each section.

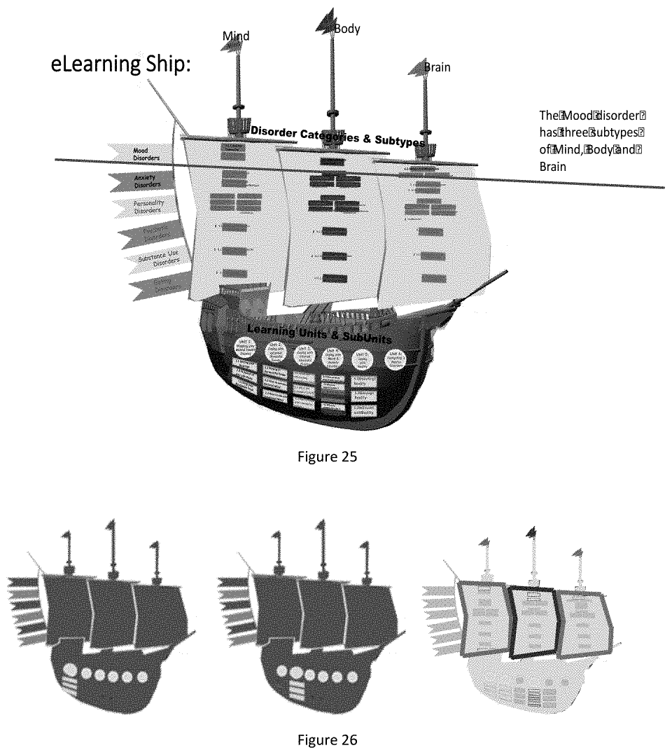

The Crows' Nest is configured to be large enough to have labelling Mind, Body and Brain. This is to replace the current portholes graphic, and preferably the color pallet and line drawing design approach as shown in FIG. 25. Hull is configured to be modified so that the portholes become rectangular apertures with rectangles not circles. Labelled Unit 1, Unit 2, Unit 3, Unit 4, Unit 5, Unit 6 is shown in FIG. 25. The eLEARNING Ship will then need to be explained, so when the user clicks on a part of the ship and explanation comes up as shown below. This should be aligned to the design of the Learning Units so there is some consistency between the CMS and LMS.

In another embodiment, a cut down version of the eLearning Ship is provided in the interface as shown in FIG. 26. On each web page or interface of the Learning Subunits a cut down version of the ship may be shown to the user where they are in the program with a user device of lower specification.

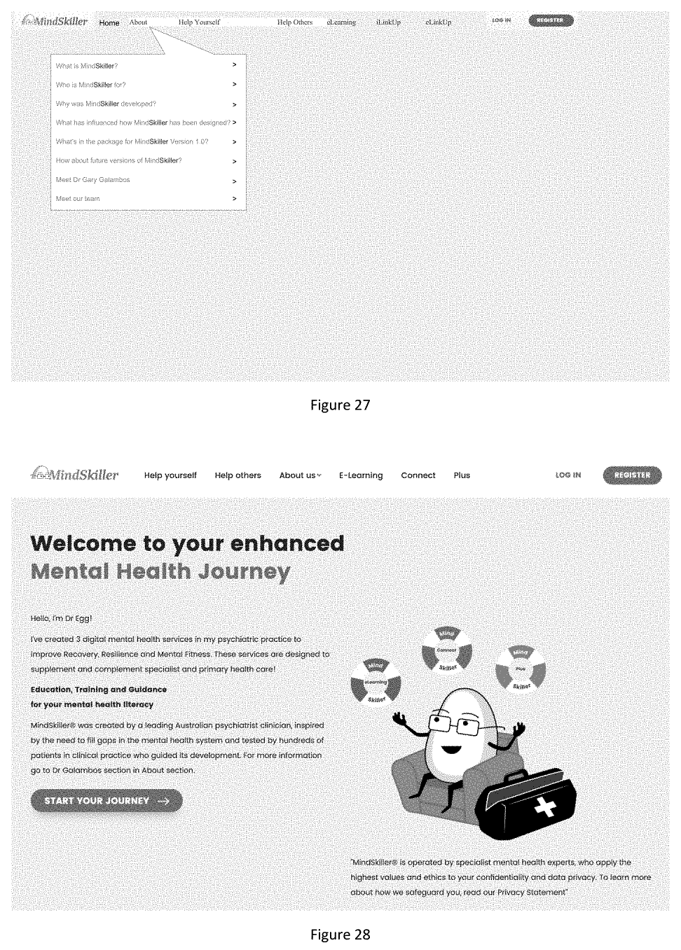

Each tab now has a drop-down menu as shown in FIG. 27.

Referring to FIG. 28, there is shown an example of the Home page of the system of a preferred embodiment of the present disclosure. The Home page now starts with the three key services of MindSkiller, which are represented by the three Life Preservers of eLEARNING, CONNECT and PLUS. These Life Preservers are also on the side of the Tugboat assistant.

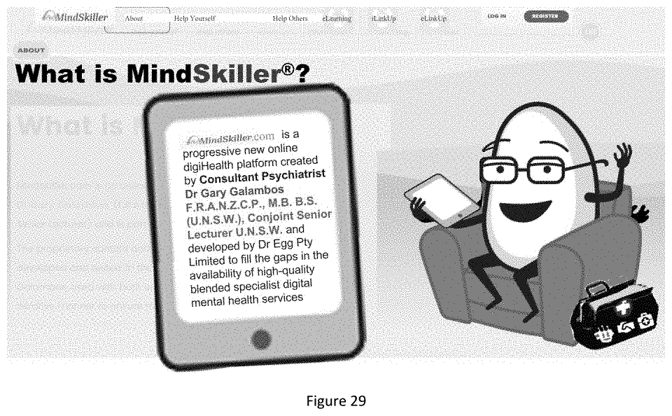

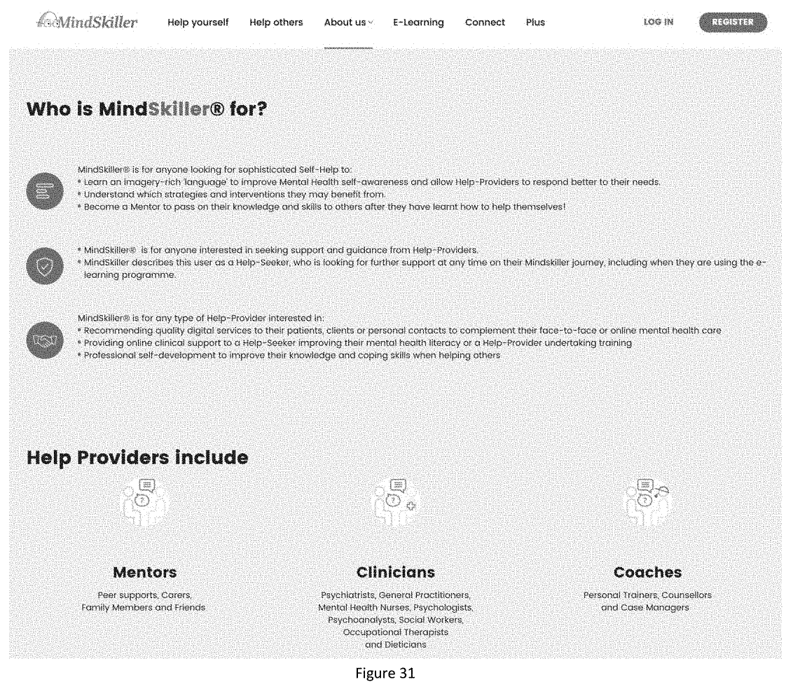

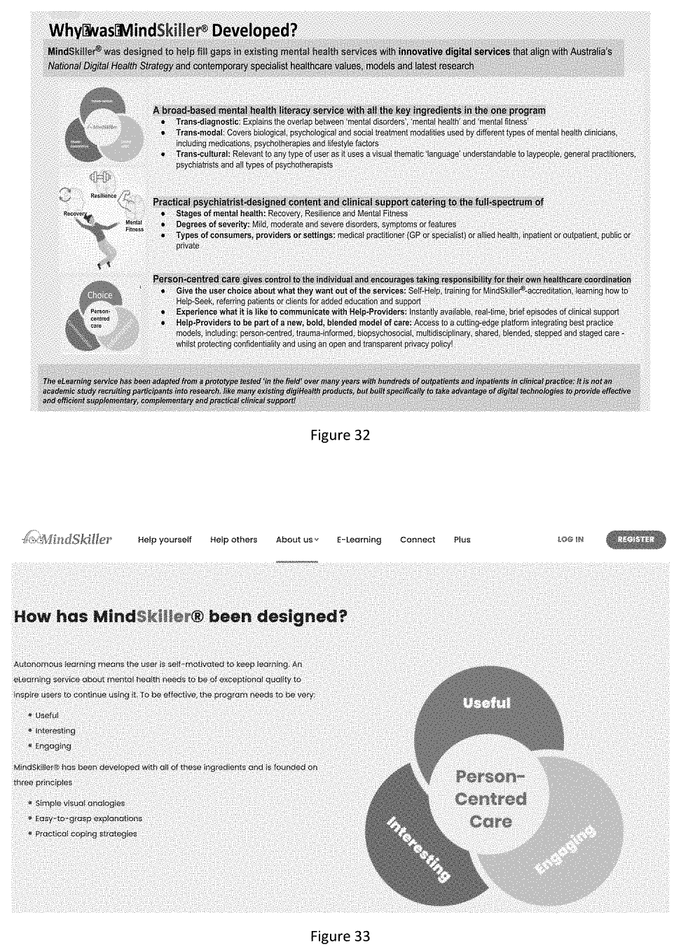

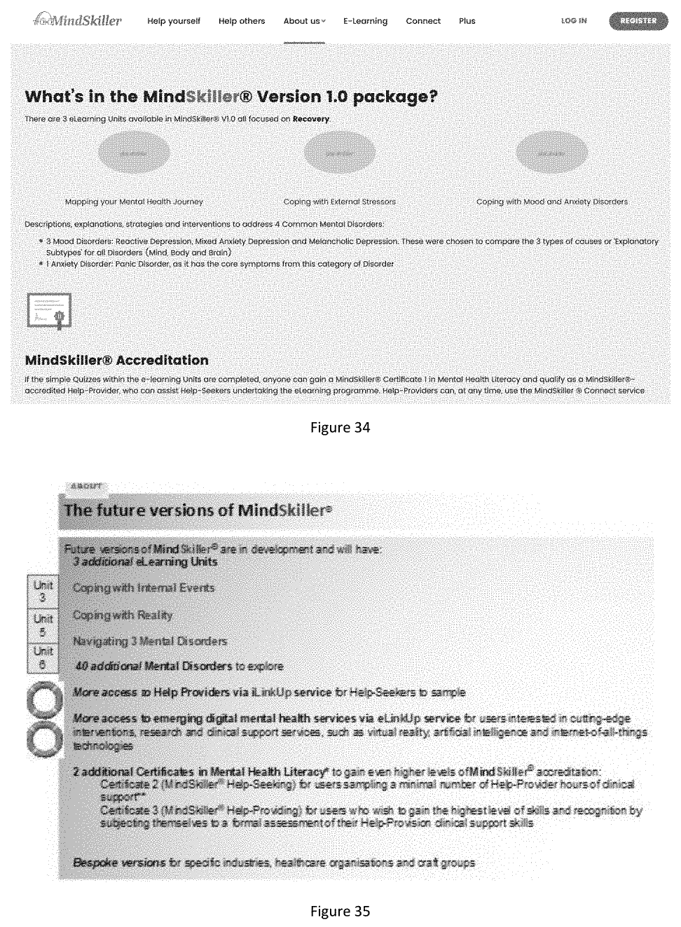

In one embodiment, a new page layout is provided for the first 6 pages of the ‘About: section, although some of the graphics that have been developed as shown in FIGS. 29 to 35 can be used.

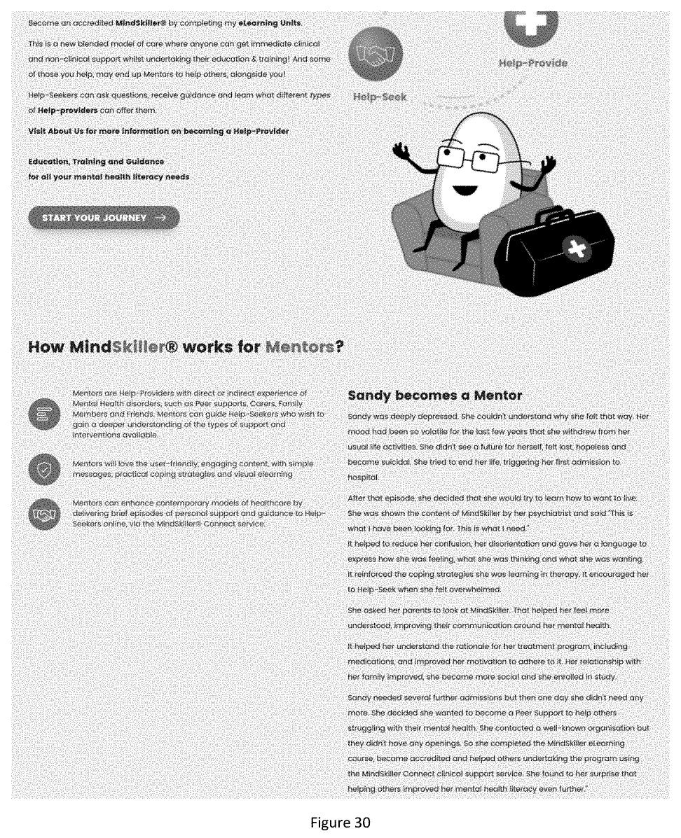

A new page layout for the How MindSkiller Works section is shown in FIG. 30. There are three types of Help-Provider—Clinicians, Mentors (such as Peer Support) and Coaches.

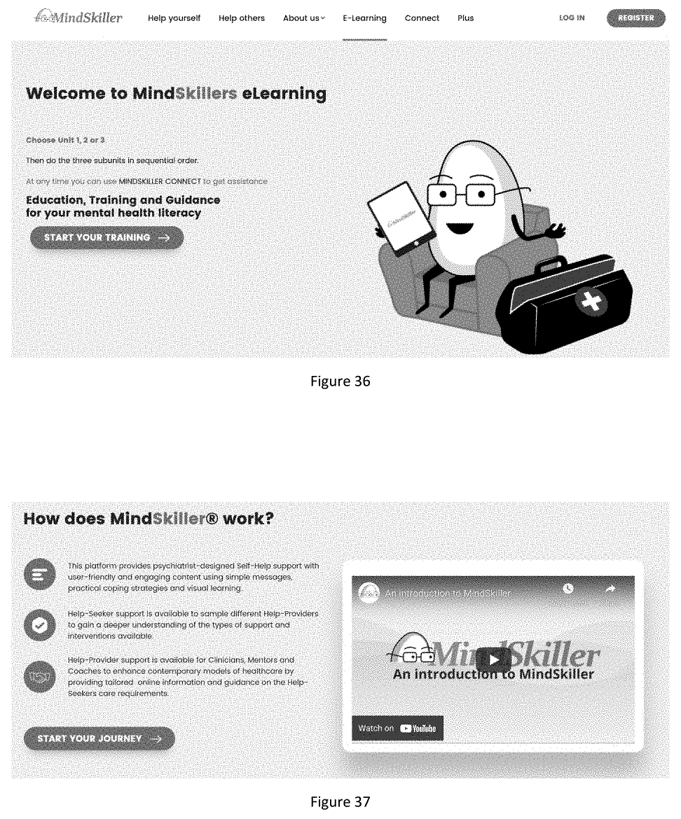

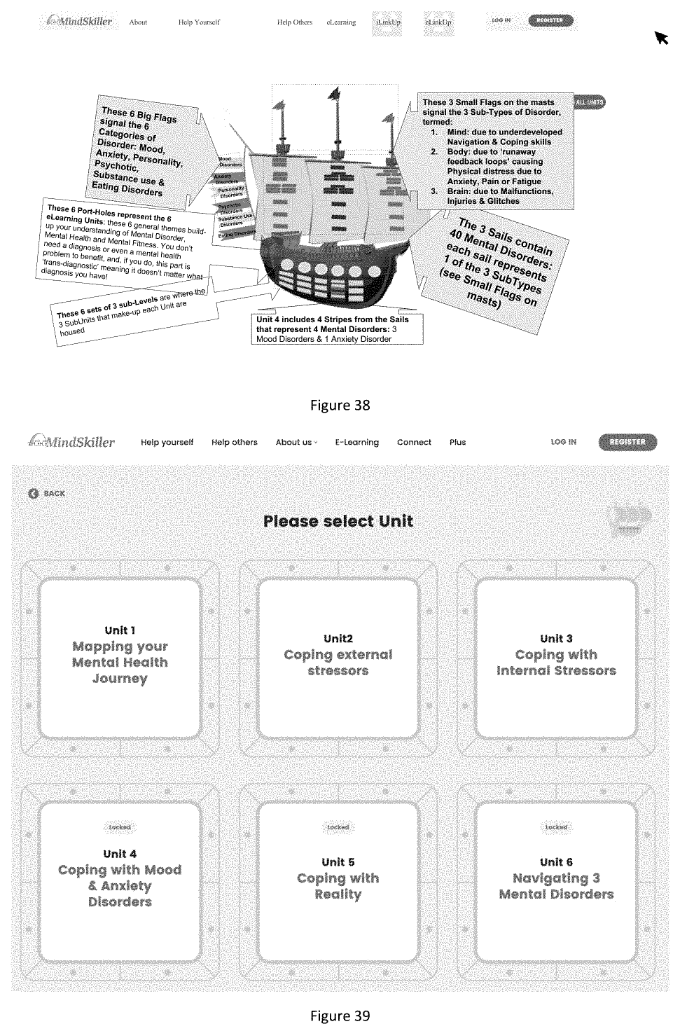

In a preferred embodiment of the system of the present disclosure, there is provided an eLEARNING Landing Page as shown in FIGS. 36 to 38. An Overall Contents Page for the eLEARNING is configured to be designed, with some hover over copy to explain each key part of the eLEARNING Ship. This is designed for welcoming users to the MindSkiller eLEARNING service. In one embodiment, there is provided 3 Units selectable by a user from: Unit 1, Unit 2 and Unit 3. The user may undertake them in any order but it is preferable and recommended on the display of the interface when starting the journey at the beginning. When a user chooses either Unit 1 or 2, the system will restrict the user option to undertake its 3 Subunits in sequential order to complete the Unit before moving to other functions of the system. At any time, a user may click on the CONNECT life preserver on the Tugboat to gain support and guidance from an accredited Help-Provider.

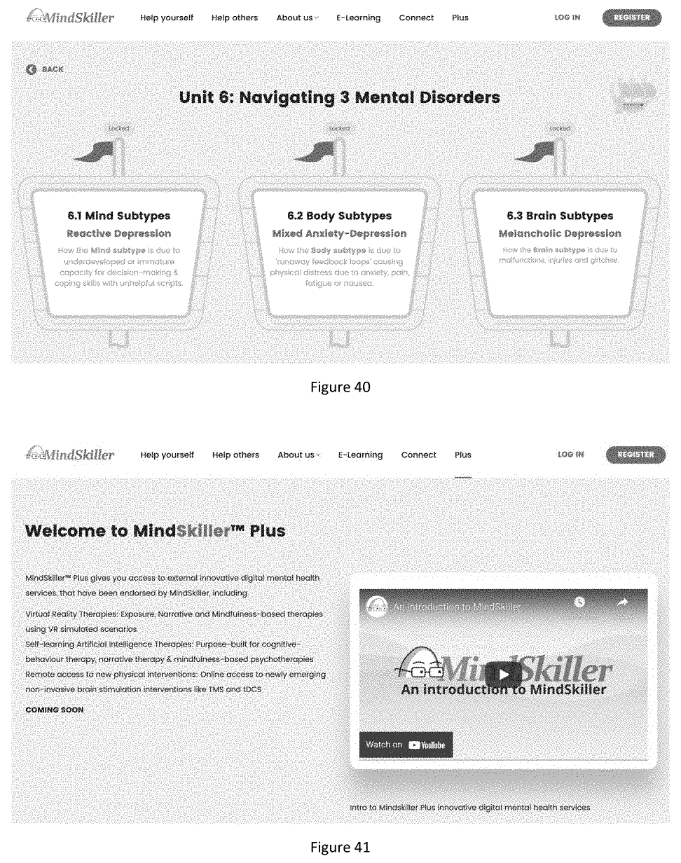

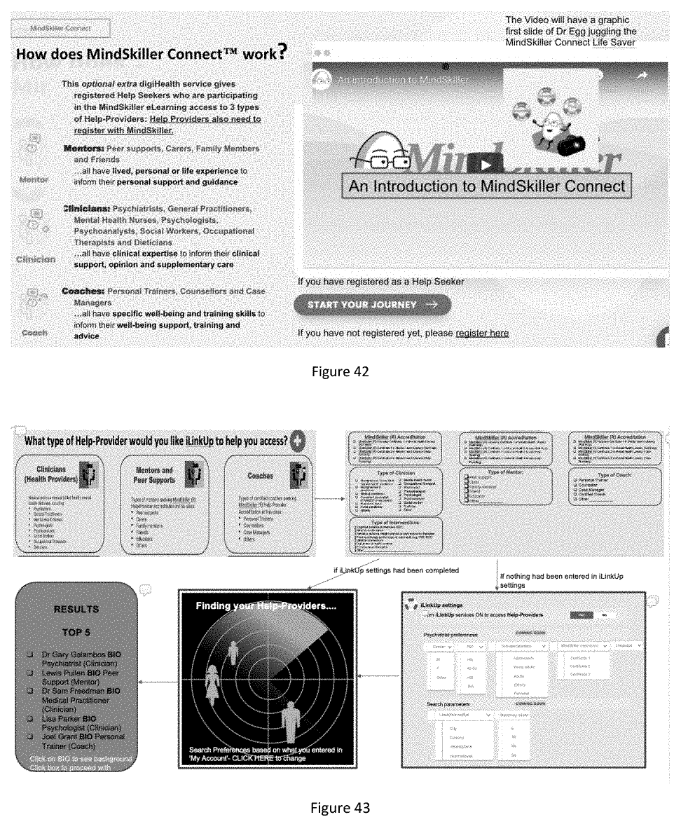

FIG. 39 and FIG. 40 shows an eLEARNING gateway interface. The eLEARNING gateway is designed with rectangular and oval portholes. FIG. 41 and FIG. 42 shows an example interface of the Tugboat of the system of an embodiment of the present disclosure. FIGS. 42 and 43 illustrated the CONNECT web page interface narrative of an embodiment of the present disclosure.

The objective of the MindSkiller MVP Beta V1.0 is to test the content and online journey with a selected group of users, both Self-Help users and Help-Providers.

The technical strategy of the MindSkiller product of one embodiment of the present disclosure is to take an uncoupled, Cloud based, SaaS approach to the pilot. These platforms will have limitations in terms of how the content is displayed, functionality and the level of customization. However, the business benefits outlined above—of speed to market and lower costs—outweigh these initial limitations in the pilot.

At the end of the pilot, it can be determined whether the platform needs to migrate to a bespoke platform, specifically coded for the MindSkiller user experience and functionality. Ideally this platform design can accommodate the next versions of MindSkiller.

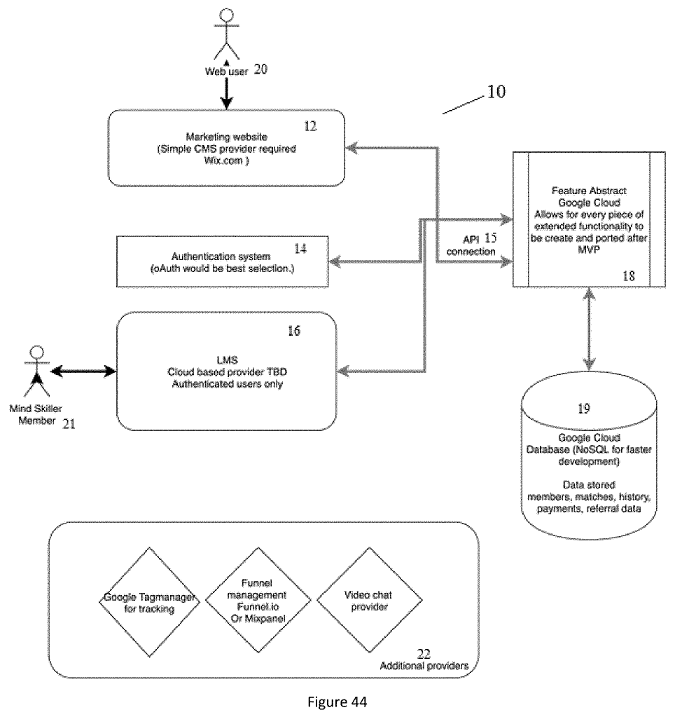

Referring to FIG. 44, there is shown a high-level schematic diagram breaking the tech build down to a five-key subsystem of the MindSkiller system 10 of an embodiment of the present disclosure:

Marketing Website 12

Learning management system (LMS) 16

Custom MindSkiller services and storage 19

Bolt on SAAS services 18

Moveable Authentication framework 14

Each of these high-level systems will be connected by API's (Application Programming Interface) or Middleware, reducing the amount of custom code that needs to be created.

The system 10 requires a simple, easy-to-use, cheap, fast-to-setup CMS (Content Management System) for the front-end marketing website. The preferred SaaS platform is self-hosted content management system, which has prebuilt templates.

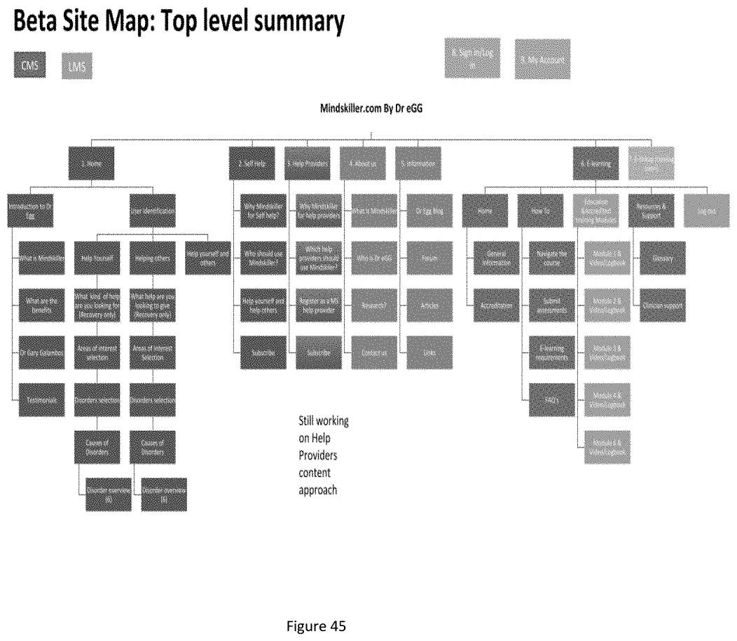

An initial site map as shown in FIG. 45 was drafted build out the initial marketing website 12.

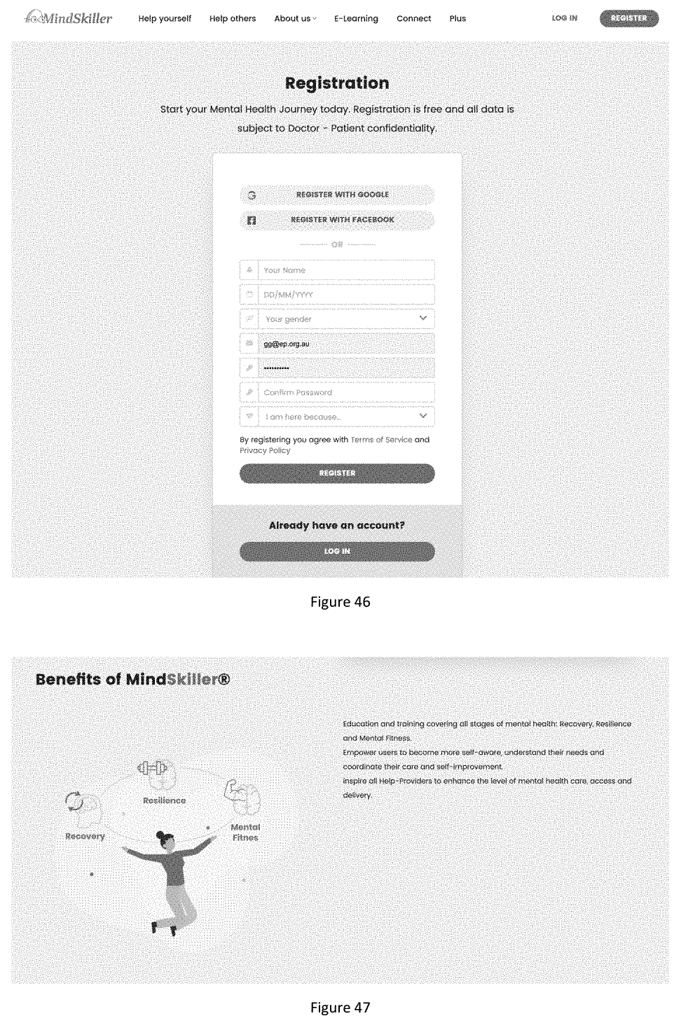

As self-hosted content management system is user-friendly, the site can be mocked up and easily changed from a look and feel perspective. The initial pages will be based on the website template design as shown in FIG. 46.

The custom-built assets can be pulled in as vector graphics as they are completed. It is also envisaged that stock footage, via a subscription with Shutterstock, can be used to also reduce the design costs for the pilot.

Learning Management System 16

In one embodiment, the LMS of the present disclosure is built on an open-source learning management system to provide SaaS LM functionality. From a tech point of view though, the LMS of the present disclosure must provide API's that allow MindSkiller to get data in and out of the LMS when required, as well as webhooks that can be used as triggers for automation. One of the features that may be provided by the LMS of the present disclosure is the possibility of using a separate MindSkiller Authentication framework 14. This would enable MindSkiller to store its users in a separate database, to allow other custom events and reports. Having the user profiles and authentication managed by MindSkiller from the start ensures that any migration issues will be reduced (migration of user profiles/credentials from one system to another is always problematic when re-platforming.)

Custom MindSkiller Services and Storage

Since the Marketing Website and LMS platform will provide a fixed set of features and functionality, any additional bespoke features will need to be coded separately. This can be built as an external code interface, where communication is via APIs. For instance, this is where the following functionality can be built:
Custom downloads
Store all usage data
Personalized Mental health management plans
Referral mechanics
Custom dashboards

In one embodiment, bespoke features system may either be asp.net code or node.js, using a non-SQL database for storage, all hosted with Cloud services. Having as much of the data in a system that MindSkiller controls, gives future platform flexibility and the ability to build further bespoke solutions. The Cloud Services implement in the present disclosure may grant available to start-ups that could allow MindSkiller to provide an economic service. The Cloud Services may be Amazon Web Service (AWS), Google Cloud (GC), Microsoft Azure depending on the target customers and processing requirements.

Bolt on SAAS Services

This Bolt on SAAS services 18 of an embodiment of the present disclosure may include:
Analytics services for tracking inbound and outbound service requests;
Business analytics application for funnel management
service and platform integration
Video chat provider
Support service integration (e.g., things like Zendesk or Intercom)

Moveable Authentication Framework

In one embodiment MindSkiller provides an independent user authentication system that can snap into other platforms. It is similar to the way that a user can “Login with Facebook” or “Login with Google account” to third party sites. The core login and user profile is stored in a place controlled by MindSkiller, but allows users to login to the Marketing, LMS or future dashboards using that same login.

If oAuth or similar standard is followed, this moveable authentication framework would be possible, ensure that user data is in MindSkiller's control and this data would not be required to migrate when changing to another authentication system.

To determine the final budget for the tech build, the journey map of the present disclosure is translated into a key features/functionality list. This will then provide a more detailed specification for the Custom services (3) and bolt-on services (4).

The custom services will be the major cost component because of developer time. However it is envisaged that the SAAS platforms will provide most of the functionality for one embodiment.

Decoupled are two or more systems that are able to transact without being connected or coupled. A decoupled system allows changes to be made to any one system without having an effect on any other system. A decoupled architecture is where the different components/layers that make up the system interact with each other using well-defined interfaces rather than depending tightly on each other.

FIG. 47 shows the two approaches that may be implemented in the system 10 of an embodiment of the present disclosure. In a coupled approach there will always be integration challenges of getting the two systems to talk to each other as they are two different sites with different code structures. Most LMS are built in php not .net—so are difficult to customize. For example, a Moodle LMS has hard coded navigation, which again makes it difficult to deliver a bespoke solution for MindSkiller. It has been around for 18 years so it is relatively old technology. It may be used for the beta testing but anticipate the system 10 may migrate from this platform in the another LMS platform.

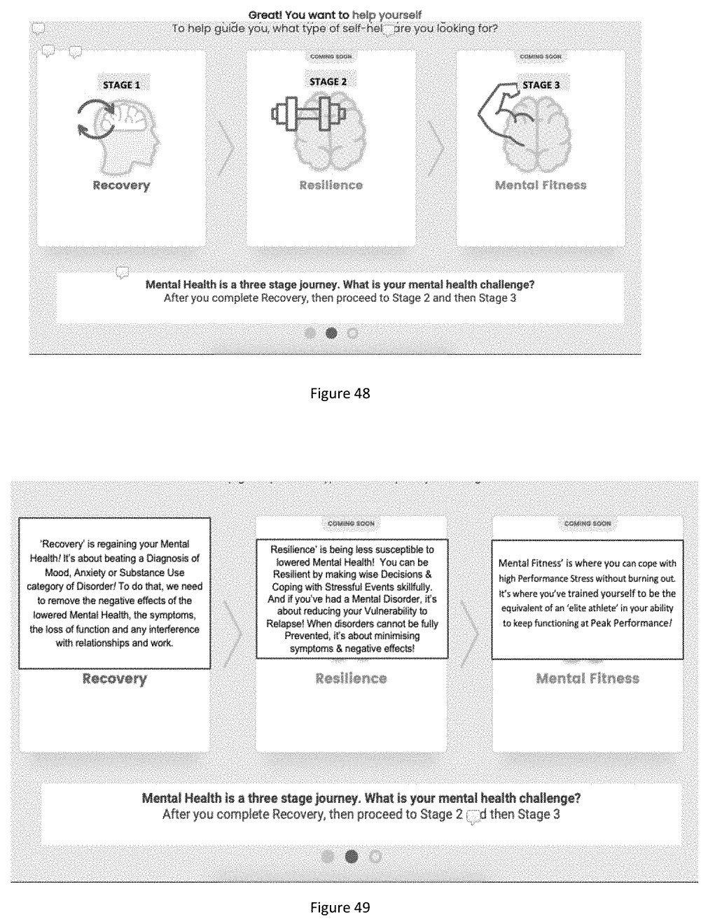

FIGS. 48 and 49 show two different coupled approaches that visualize some of the challenges of integration and ultimately migrating away from either platform.

If at any stage, it is required to move to a different CMS platform, or change external functionality, a major rebuild of the platform may be required. As long as an LMS supports LTI, the current system 10 can be migrated to another LMS e.g., SAP e-Learning System.

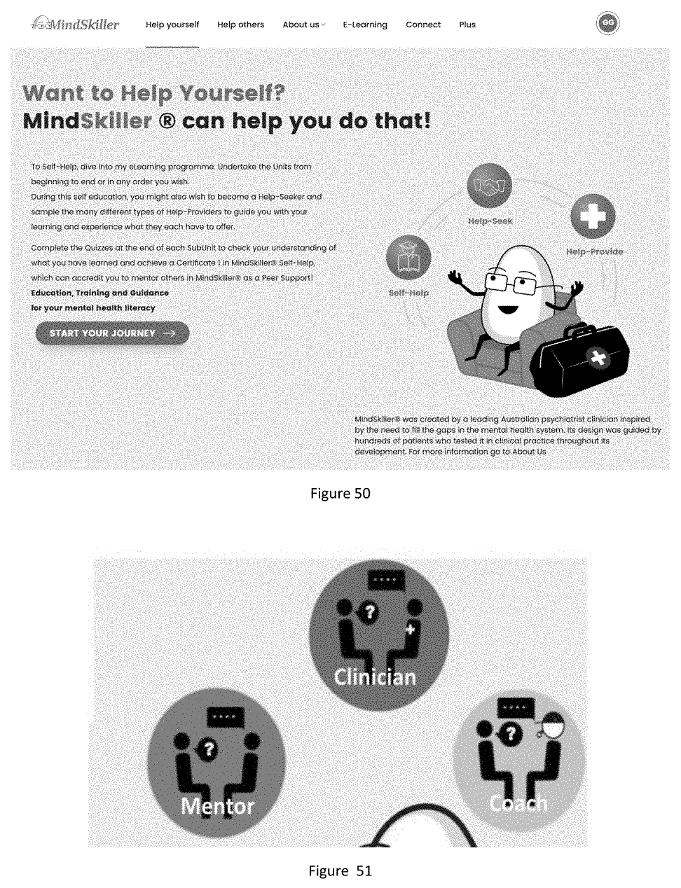

In a decoupled approach as shown in FIG. 50, each component (CMS, LMS etc.) can be plugged in and out. It is also easier to add functionality by plugging in other applications e.g., web chat feature, subscription model, online doctor booking etc.

From a user perspective this approach will also deliver the best seamless online experience. It effectively delivers one site, fully integrated. It will create one front end that is integrated with both LMS and CMS and everything else e.g., if a user is logged into a marketing website they are also logged in LMS. It will make it easier to have a single user dashboard and MyAccount, and allow the system 10 to pull in LMS data into the SMS e.g., a header that informs the user of the progress on their e-Learning etc.

This approach returns to some of the original thinking on architecture captured in the front of this architecture e.g., separate database, authentication system, bespoke software for customized feature etc., which gives us the flexibility going forward.

The decoupled approach recommended is to use a modern software development framework, such as Lavarel. The modern software development framework for the embodiment of the present disclosure is designed to provide robust features, simple and secure authentication mechanism, elegant syntax pattern, well-thought homestead, and affordable. In one embodiment of the present disclosure, a PHP framework is used for implementation. With its various useful features, it lets developers build their websites fast and without the struggle. Also, it's very fluent, user-friendly and easy-to-learn and understand. Preferably, the software framework used for the present disclosure contains a web application framework with expressive, elegant syntax. Laravel attempts to take the pain out of development by easing common tasks used in the majority of web projects, such as authentication, routing, sessions, and caching.

The modern software framework in one embodiment of the present disclosure combines the very best of different modules of web frameworks, including frameworks implemented in other languages, such as Ruby on Rails, ASP.NET MVC, and Sinatra. The modern software framework in one embodiment of the present disclosure is made accessible, providing powerful tools needed for large, robust applications. A superb inversion of control container, expressive migration system, and tightly integrated unit testing support gives MindSkiller the tools to build the platform and applications.

This will allow the system 10 to start with a low cost LMS, and then migrate toward a more sophisticated version post-beta. It will also allow us to add extended functionality and features quickly. It also allows the system 10 to use a CMS that is still user-friendly e.g., October CMS. When at some stage, the system 10 of the present disclosure needs to use a new LMS or CMS, the content can be lifted and shifted (as long as the scorn content schema can be used).

Regarding the beta testing stage of the system 10 of an embodiment of the present disclosure, the testers may include Self-Help Users, Help-Seeking Users, and Help-Provider Users.

1) Self-Help Users—those just testing the MindSkiller eLEARNING Service for self-education purposes. They may wish to also undertake the training option (by completing all 3 Units including the 3 quizzes that each contain within those Units that the user has the choice to undertake or to skip) that will yield them with a Certificate (“MindSkiller Certificate 1 in Self-Help Mental Health Literacy” or “Certificate 1”), which they could use as accreditation and use CONNECT to help others as a Peer Support type of Mentor.

2) Help-Seeking Users—those who wish to utilize the MindSkiller CONNECT service whilst they are undertaking the eLEARNING. That includes completing the relevant sections of My Account where the user provides:

a) authorization to send Progress Reports to external help-providers—so they will need to provide some details like the provider's name, email and type of provider (these providers do not need to be accredited)

b) configuring the search parameters of the brokerage service to search for accredited help-providers—those who have competed the training and received at least one Certificate, which will be either Units 1 and 2 for a Cert 1 (any Help-Provider) or Unit 3 for a Cert 2 (any Help-Provider who has completed a Cert 1 or any qualified clinician may attain just a Cert 2) in MindSkiller V1.0.

c) providing information necessary to pay the help-provider (which includes uploading referral documents, Medicare item numbers and the provision for private health insurance details or the details of other types of insurers as well as there may be funding from any or all of those services in the future—if the user wishes to take advantage of those potential funding sources available in Australia).

3) Help-Provider Users—these are users who have undertaken training using eLEARNING and have gained a Certificate. They will be entitled to log into CONNECT and provide clinical support to users who are Help-Seekers (whose using eLEARNING and wish to gain up to 30 min of clinical support using a video-conferencing Telehealth functionality).

In another embodiment of the system 10, it will enable the Help-provider to be paid for their services but beta-testing will not have a payment facility. Some beta-testers will be entitled to bill Medicare for their help provision—these will predominantly be clinicians who can access Telehealth Medicare item numbers if the Help-Seeker has been referred to them and they satisfy Medicare eligibility.

Others may provide help as volunteers for their services or as a barter arrangement for being involved in the beta-testing process e.g., users will not be paying any subscription rates for use of the software to gain education, training and accreditation so they may consider it a ‘fair exchange’ for them to provide any equal amount of time to help-provision that they expended in free use of the software for their benefit.

It is anticipated that in future versions, Help-Seekers will pay Help-providers rates based on the type of Help-Provider, some of which may be able to be subsided or paid for in full by a funder (e.g., bulk billed by Medicare or paid for in part or in full by a health insurer, worker's compensation insurer, corporation, educative facility, non-government organization, health facility or other entity).

MindSkiller pragmatic infotainment rich and engaging eLEARNING service is adapted to provide all user types in the one product (self-help, help-seeking and peer support mentors; other types of mentor, clinician and coach help-providers for training, instant online support and CPD) with some highly novel features, including:

(i) Autonomous learning principles, an innovative true visual learning approach with visual analogies, role play with conversing characters and avatars enabling a minimization of technical words and jargon;

(ii) Continuous user interactivity, personalizing opportunities and embedded quizzes makes training pressure free and the service more engaging than iCBT type courses;

(iii) Specialist psychiatry-grade ‘explanatory categorizations’ incorporated in a syllabus that complements and enhances existingly recognized descriptive classifications;

(iv) Design to supplement, complement and make more efficient and safe existing face-to-face general practitioner, specialist and allied health mental health care.

The system 10 of the present disclosure is adapted to provide online access to innovative digital mental health services to supplement and complement existing general practitioner and specialist care with cutting edge interventions and technological developments, such as AI, VR and neurostimulation therapies.

The system 10 of the present disclosure as shown in the conceptual drawings in FIG. 47 is adapted to provide TeleHealth consultations. As such, there is no mental health consumer-provider brokerage service using clinical care pathways for education, training and accreditation utilizing contemporary models and introducing a new model to support help-seeking mental health literacy in the form of ‘clinical support’ and guidance experiences.

The specialist-grade virtual healthcare system infrastructure provides clinical care pathways that are supplementary and complementary to existing face-to-face care, incorporating multi-modal, trans-diagnostic, multi-user type services, and has hospital-grade healthcare standards, regulations and accreditation processes to maintain quality control for all users.

The system can assist organizations to dramatically reduce financial and productivity losses due to mental health disability;

Bespoke industry-specific versions and business plans can reduce the financial costs of health insurance members with severe personality disorders who maladaptively cope using unhealthy dependency patterns;

The system can improve the effectiveness, efficiency and ease of access for general users by utilizing a specialist-grade mental health literacy service that is broad in scope and was built to fill the gaps from a psychiatrist's perspective who has had a broad professional skill set as a clinician, educator, reform advocate and private sector service development leader.

The benefits of education and training covering all stages of mental health are shown in the conceptual diagram of FIG. 48, which contains Recovery, Resilience and Mental Fitness.

The mental health literacy education, training and guidance can empower users to become more self-aware, better understand their needs and better coordinate their care and self-improvement. The method can inspire Help-Providers who are interested in providing clinical support to fill gaps and enhance mental health care delivery.

The system 10 allows users to conduct self-help activity using the eLEARNING service to undertake the Learning Units from beginning to end or in any order. During their self-education, the user may learn to become a Help-Seeker and sample the different types of Help-Providers to experience what they each have to offer.

The three stage concepts of education and training are shown in FIGS. 49 and 50.

The system 10 is designed with three services for anyone interested in ‘Helping Themselves’:

(i) Self-Help support with user-friendly and engaging content using simple messages, practical coping strategies and visual learning;

(ii) Help-Seeker support to sample different Help-Providers to gain a deeper understanding of the types of support and interventions available; and

(iii) Help-Provider support for clinicians, mentors and coaches to enhance contemporary models of healthcare by providing brief episodes of online support and guidance to Help-Seekers.

In relation to Self Help support, the system of the present disclosure is designed for users interested in sophisticated Self-Help to:

(a) Learn an imagery-rich ‘language’ to improve self-awareness and allow Help-Providers to respond better to their needs;

(b) Understand which strategies and interventions they may benefit from; and

(c) Become a Mentor to pass on their knowledge and skills to others after they have learnt how to help themselves.

In relation to Help-Seeking, the system 10 of the present disclosure is designed for anyone interested in seeking support and guidance from Help-Providers. These categories of users are regarded in the system 10 of the present disclosure as a Help-Seeker, who is looking for further support at any time on their MindSkiller journey, including when they are using the e-LEARNING service.

In relation to Help-Providers, the system 10 of the present disclosure is also designed for any type of Help-Provider interested in:

(i) Recommending quality digital services to their patients, clients or personal contacts to complement their face-to-face or online mental health care;

(ii) Providing online clinical support to a Help-Seeker improving their mental health literacy or a Help-Provider undertaking training; and

(iii) Professional self-development to improve their knowledge and coping skills when helping others.

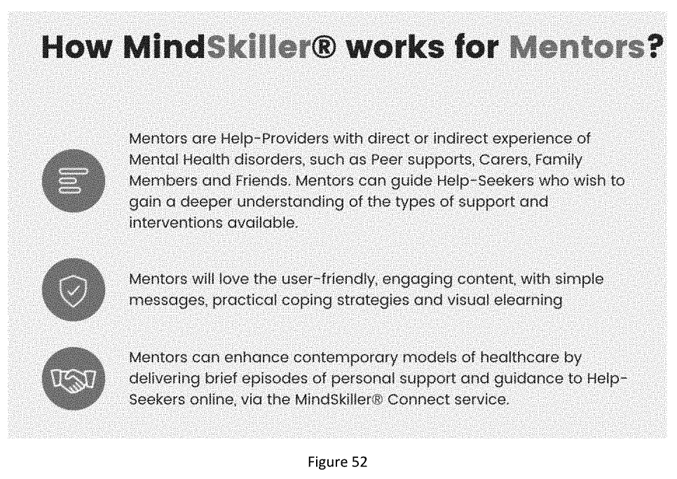

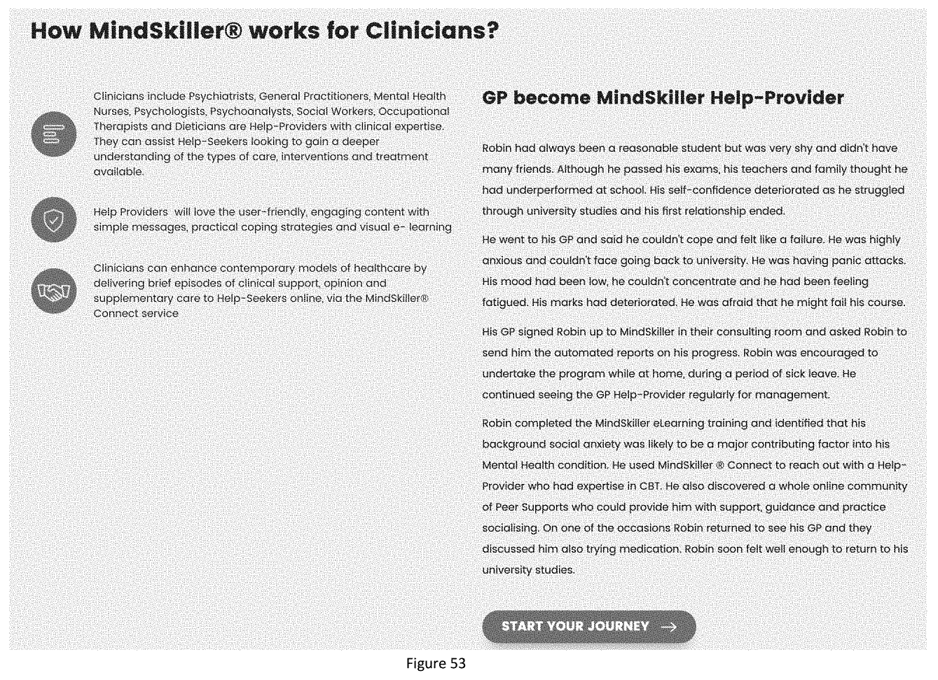

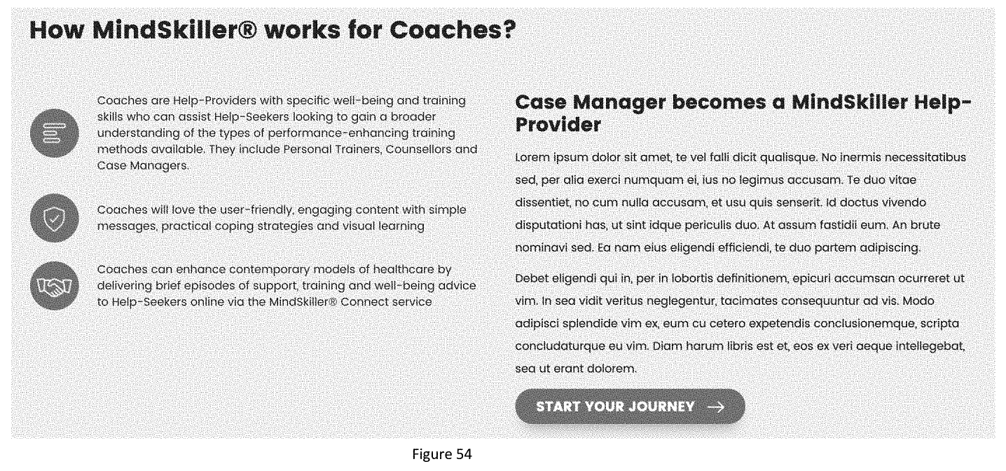

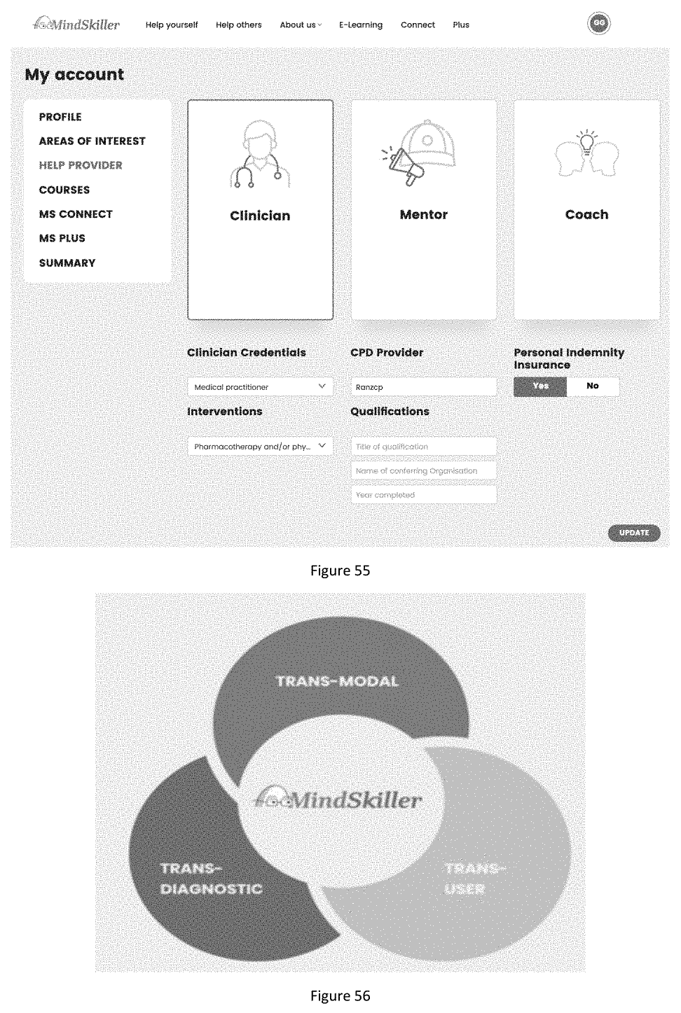

FIGS. 51 to 55 shows the conceptual drawings of the Help-Provider groups. The Help-Provider groups aims to

(i) help others' resilience, recovery and Mental Fitness by becoming a MindSkiller Help-Provider—such as a Clinician, Coach or Mentor;

(ii) become an accredited MindSkiller clinician, mentor or coach by completing required eLEARNING Units;

(iii) provide a new blended model of care where users can get immediate clinical support whilst undertaking their education and training and equip those users who may become Mentors to help others; and

(iv) provide a platform to ask questions, receive guidance and learn what different types of Help-Providers can offer them. The type of support facilitated is innovative in being user-determined episodes of psychoeducational guidance based on the user's needs in the moment whilst undertaking the mental health literacy program (expected to be under 30 min), rather than the typical psychotherapy model of consultations associated with a time duration predetermined by the provider (that is usually over 30 min). This Telehealth-based method of mental health literacy support and guidance thereby expands and enhances traditional mental health care models.

In an embodiment of the present disclosure, the system 10 provides a virtual health service for accrediting users, as such the system 10 is adapted to ensure a reasonable quality control. The system 10 is adapted to combine the quality of control of the training with the actual eLEARNING service itself, which is a novel way to provide a “virtual hospital/community mental health service.” The system 10 simulates how hospitals work in that the structure of the hospital's wards, admission and discharge processes, accreditation of clinical staff, and clinical programs all together provides a quality product. That's what's missing from most online digital mental health services—a sophisticated underlying “clinical care pathway.” That's what the system 10 of the present disclosure is developing. A virtual “clinical care pathway” to both enhance recovery, the development of resilience and mental fitness that is truly complementary and supplementary to existing in-person care pathways and improves efficiency and the user experience of their mental health journey. This will also reduce actual physical contact and minimize risk of spreading virus during pandemic time. And there are attributes that make it superior to any other digital mental health service as it incorporates all the models that are considered modern, cutting-edge and most ethical in psychiatric mental health care—especially truly putting the user in the center. In addition to the mentors, the system 10 is adapted to check external qualifications and insurance status for certain class of users such as clinician (or for some certified coaches) who have the credential to do so as shown in FIG. 56. The system 10 may connect to a third party verification service to retrieve the credential.

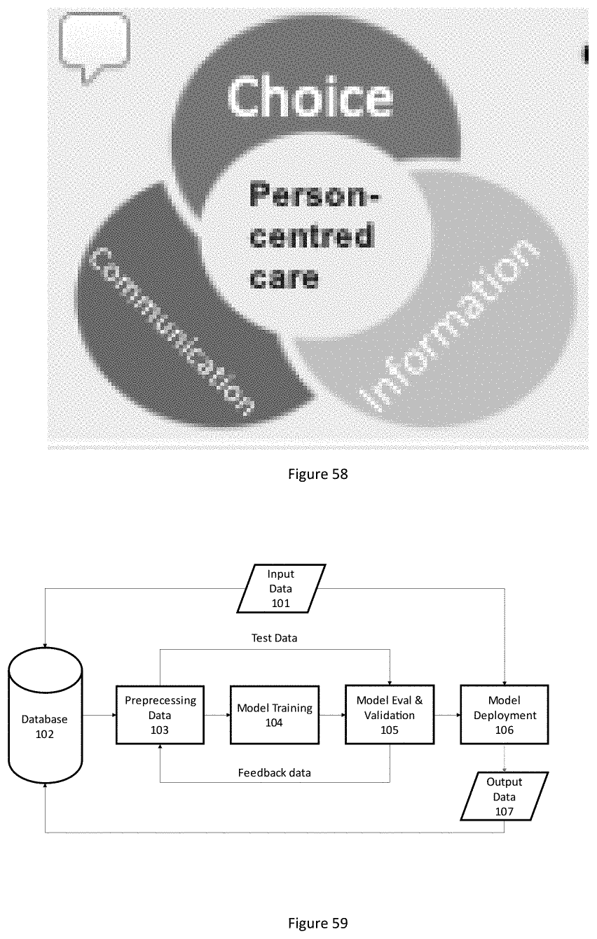

FIGS. 57 and 58 shows the 3 categories of gaps in existing digital mental health services.

In one embodiment of the present disclosure, the system 10 provides a broad-based mental health literacy service with all the key ingredients in the one program including, but not limited to,
Trans-diagnostic: Explains the overlap between ‘mental disorders,’ ‘mental health’ and ‘mental fitness’
Trans-modal: Covers biological, psychological and social treatment modalities used by different types of mental health clinicians, including medications, psychotherapies and lifestyle factors
Trans-cultural: Relevant to any type of user as it uses a visual thematic ‘language’ understandable to laypeople, general practitioners, psychiatrists and all types of psychotherapists

The practical psychiatrist-designed content and clinical support provided by system 10 is designed to cater to the full-spectrum of
Stages of mental health: Recovery, Resilience and Mental Fitness
Degrees of severity: Mild, moderate and severe disorders, symptoms or features
Types of consumers, providers or settings: medical practitioner (general practitioner or specialist) or allied health, inpatient or outpatient, public or private

FIG. 59 shows a part of an artificial intelligent engine of the system of FIG. 1.

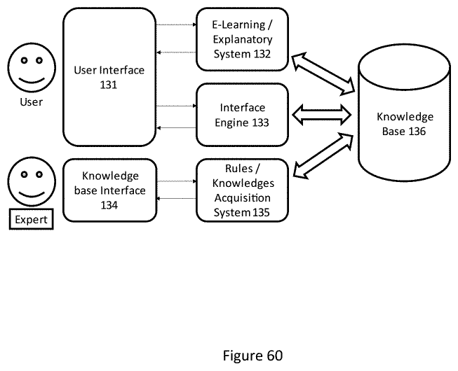

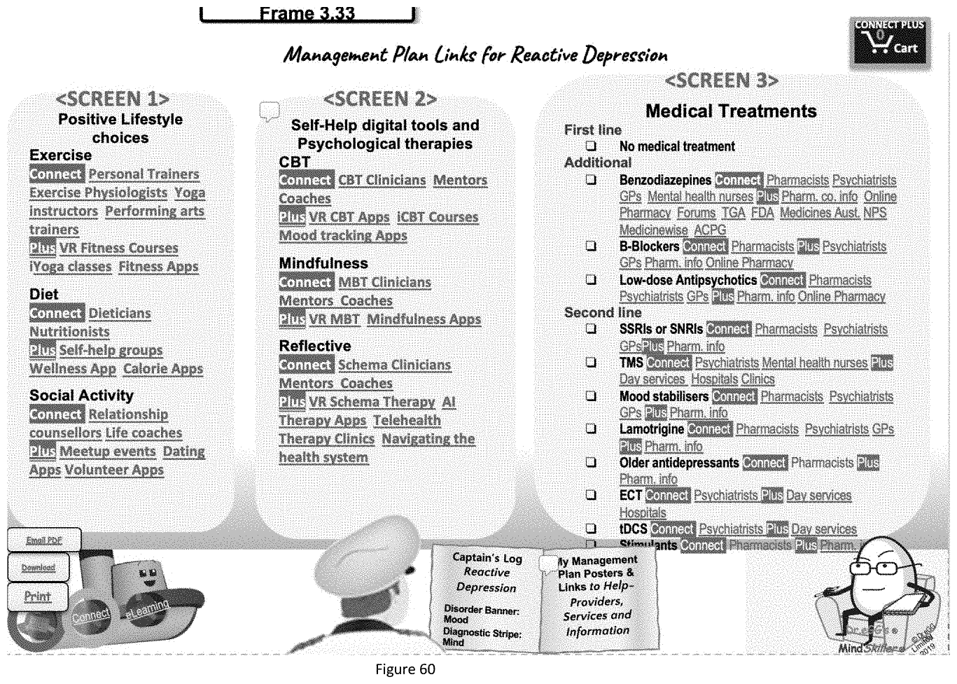

FIG. 60 shows a person-centered care service provided by system 10, which gives control to the individual and encourages taking responsibility for their own healthcare coordination. The system 10 is adapted to:
Give the user choice about what they want out of the services: Self-Help, training for accreditation, learning how to Help-Seek, referring patients or clients for added education and support;
Experience what it is like to communicate with Help-Providers: instantly available, real-time, brief episodes of clinical support; and
Help-Providers to be part of a new, bold, blended model of care: access to a cutting-edge platform integrating best practice models, including: person-centered, trauma-informed, biopsychosocial, multidisciplinary, shared, blended, stepped and staged care—whilst protecting confidentiality and using an open and transparent privacy policy.

Reference is now made to FIGS. 1 to 61. FIG. 61 discloses a visualization of Management (Plan) Links where the choices the user has made to explore management options are linked by CONNECT and PLUS services to the database of accredited mental health providers and digital mental health services.

The system 10 is designed with three main ingredients or principles for the eLEARNING service:
Simple Visual Analogies
Easy-to-grasp Explanations
Practical Coping Strategies

In one embodiment of the present disclosure, the system 10 provides a practical and evidence-informed psychiatrist-developed online education program that grows sophisticated mental health literacy in all user types, utilizing an intuitive theme-based ‘visual language’ based on visual analogies, metaphors and representations, jargon-free layman's language and simple messaging, that is designed to be complementary to face-to-face clinical care and support and other types of online clinical care and support.

The MindSkiller system 10 of the present disclosure gives Help-Providers an underlying psychoeducational landscape/structure/platform through which to assist and support ‘Help-Seekers’ and encourages them to become accredited to become a ‘Help-Provider.’ This includes the features that:
In one embodiment, for any users seeking a Certificate 3, it will be mandatory to undertake specified amounts and categories of Help-Seeking.
The educational tool gives them the information they need to not only Self-Help but to Help-Seek the right type of further assistance. That is a novel method to address a cultural change that has been promoted in all healthcare but particularly mental healthcare—putting the service user in the center and encouraging consumers to take responsibility for their own healthcare coordination.
MindSkiller system 10 provides a new model of care because it facilitates and supports the use of briefer than usual online consultations that are user-instigated for specific goals or purposes or to answer specific questions or concerns that may arise during their use of the program. This is in contrast to clinical care typically dictated by the Help-Provider's model of care.
The system 10 of the present disclosure puts the user in charge to dictate what and who they want and why. It encourages consumers to take responsibility for their health and to become the central coordinator of their healthcare, utilizing mentors, clinicians and coaches to support them. It is particularly important to encourage a model like this for e-mental health to protect the user from being exploited and wasting their time, energy and money on low quality or detrimental assistance.

The present disclosure of system 10 is adapted to provide a clinical service focusing on empowering the user to become their own coordinator of their mental health care.

The system 10 gently encourages users to do what they need to do to get the results/outcomes they are looking for, which includes Help-Seeking—and the program facilitates them doing that both with their existing external Help-Providers who can receive a progress report if the user wants that and their provider is interested/able to accommodate.

In one embodiment, the system 10 is adapted to connect to a printing service or printing server where the user can print a hardcopy report and take it themselves to their provider. It is useful for users to become familiar with their MyAccount being the single destination within the system 10 to get an overview of all their data and progress on the platform of system 10, be able to print out material from the e-LEARNING service, including their answers—as well as sending a summary report to their Help Providers. The user can access MyAccount to do this, as this could be challenging to initiate from the LMS. For example, Campaign Monitor—the SaaS plug in used in the system 10 to manage and track emails if integrated into the CMS not the LMS. MyAccount is in the CMS. In another embodiment, the MyAccount is in the LMS.

The system 10 is adapted to step through the process of how a summary report of the user's journey through the e-LEARNING service can be sent to a Help Provider in secured form to protect the sensitivity and integrity of the data. There will need to be a number of checks in this journey so ensure that the user really does want to send the date.

In one embodiment, the system 10 of the present disclosure the CONNECT By-Laws can guide accredited Help-Providers what to do if they come across a Help-Seeker who is acutely distressed or is perceived to be an acute risk to themselves and their management requires assistance that is outside the parameters of what the Help-Provider can do to support and guide the user during the brief episode of ‘clinical support’ within MindSkiller. As such, the system 10 of the present disclosure can help a suicidal user by using the system to divert them to appropriate mental health care/service.

In one embodiment, the MindSkiller system 10 of the present disclosure can provide structure in the chaos and ruggedness of the unorganized digital mental health service environment. For a user with a mental disorder, there is currently no quality virtual hospital-community system with clinical care pathways layed out for them.

In one embodiment, the system 10 of the present disclosure is built based, at least partially, on the ‘stepped care model,’ which encourages low-cost, easily-accessible pathways starting with online education and support as the first step and each step takes the consumer to higher levels of specialist support, guidance and care. This model is considered vulnerable to misuse if it is not part of a specialist-informed mental health system where the assessed needs of the user guide the level of care they should obtain.

In one embodiment, the system 10 of the present disclosure may provide access to specialist assessment early—that's a specialist-informed ‘early intervention’ model. ‘Stepped care’ is inconsistent with ‘early intervention’ if it is not specialist-informed. An analogy with a physical health condition is that if a user has a skin lesion that is beyond the general practitioner's skillset, the general practitioner will refer the patient to a dermatologist early to ensure it's not a melanoma, because it does not make sense to wait as that would risk it progressing to the stage that it has spread and then requires more invasive life-saving measures. The same specialist-informed ‘early intervention’ model in mental health care should be used.

The MindSkiller system 10 can solve this problem by being adapted to provide ‘stepped-care’—it can both enable Self-Helpers to help themselves without external support and also provide rapid, user-defined brief episodes of access to all types of clinicians, mentors and coaches when they wish to ‘Help-Seek.’ This is a form of ultra-rapid ‘early intervention’ that can be delivered to the users by Help-Providers who have been MindSkiller-accredited and who are familiar with the content in eLEARNING (incorporating the visual language, which it utilizes to psychoeducate consumers). The Help-Providers will answer questions posed by the users during their journey through the eLEARNING service. It can encourage brief episodes of help-seeking about specific mental health concepts and introduce them to the different types of help-seeking and Help-Providers who exist in the external world. It gives the user a way of ‘testing the water,’ ‘playing with’ and ‘rehearsing’ help-seeking. It is anticipated that this will improve their knowledge, skills and strategies for help-seeking in the external world.

In another embodiment of MindSkiller system 10, there is provided a platform that is adapted to facilitate highly collaborative forms of care utilizing case conferencing, which reflects a further core model of care utilized in quality psychiatric practice: multi-disciplinary care. Here, there is a team approach to patient care or case management. This is generally reserved for cases with more severe, complex or chronic disability.

In summary, the system 10 contains three leading models of care designed to operate at a mental healthcare system level that endorses three clinical care pathways:
‘Stepped-care’
‘Early intervention’
‘Multidisciplinary care’

In addition, the system 10 may also have six other models of care designed to support a help-seeking individual patient or consumer. These 6 ‘best practice models of individual care’ are utilized in MindSkiller system:

1. Person-centered care model to take into account the individual's subjective circumstances, emotional state and any stated preferences, and respond comprehensively, non-judgmentally and compassionately. The core principle is that they be treated ‘like a person not a number’ and minimize their feeling objectified, dismissed or that meaningful aspects of their identity or personal priorities ignored.

2. Trauma-informed care model knowing that many people with mental ill health have suffered emotional, physical or sexual trauma as an adult and/or in their childhood, which may influence their presentation, expectations and interpersonal interactions. The core principal is to try to keep in mind that many patients who might be defensive, fearful, negative and even hostile may be being self-protective as a result of learning that the world can be a cold, dangerous and hurtful place and an encounter with a healthcare provider is an opportunity to experience a different kind of response to their being in pain and vulnerable—open-minded, accepting, non-judgmental and compassionate interactions, which can help reduce defensiveness and balance them out.

3. Developing mental health literacy:
Refers to knowing, expressing and sharing words and concepts that represent feelings being felt rather than acting out behaviorally in unhelpful ways due to sensory overload, emotional dysregulation or unhelpful belief systems. Psychoeducation is universally acknowledged as being of critical importance and an essential ingredient in any clinical response to mental ill-health but it is highly variable as to how often and effectively it is delivered by clinicians, allied health and psychosocial disability support providers.
Currently available online resources have been explained to be highly factual, dry and academically oriented, making engagement poor.
MindSkiller has expanded the concept of mental health literacy to refer not only to developing a language for mental health and understanding concepts of mental health, fitness and disorder, but also to understanding how to become better ‘coordinators’ of one's mental health journey, trajectory and care provision.

4. Counselling to enhance decision-making:
Adopts the principles in the individual models of care delivery in assisting the person to make healthy, balanced and wise decisions about how they pursue their goals and how they deal with stress
MindSkiller system 10 breaks decision-making into three functional capacities to assist the self-help and help-seeking user and help-providers to utilize a visual language that enables persistent utilization of personal decision-making to practice this essential life skill and way of optimizing coping strategies.
In a way, MindSkiller provides a generic ‘guidance’ for the user that is the equivalent of ‘counselling’ when people help-seek in-person clinical care and support.
In another embodiment, the system 10 provides a more personalized capacity for the AI to recommend a particular clinical care pathway or tailored course for the user based on the information they provided about their interests and priorities. In later versions, more sophisticated types of AI (such as learning AI and modified AI purpose-built for mental health literacy or even to provide a simplified form of ‘counselling’ as opposed to just guidance) could potentially be used to provide a more sophisticated type of psychoeducation that will improve patient care and outcomes and may even provide an improved model of mental health care that is much more efficient than relying on face-to-face care alone, especially when human resources (especially psychiatrists but referring to all mental health clinicians) are very limited in supply in most parts of the world.

5. Coping skills enhancement:
Problem-solving and solution-finding
Apprenticeship-styled experiential learning and emotional self-regulation using mindfulness, self-soothing and help-seeking (when self-help is ineffective or inadequate to meet current emotional needs or to cope with current stressors)
Gaining insight or self-knowledge using a relational, dyadic and conversational process of reciprocal, analogous self-reflection promoting self-awareness, identity formation (an integrated, cohesive experience of self-identity) and self-worth.

6. Utilizing cultural resources: there is an abundance of taxpayer-funded or subsidized non-government organizations (NGOs) and private individuals and organizations that provide mental health psychosocial support and care, with a few examples being:
NDIS: funding mental health disability support and rehabilitation providers
Beyondblue
Lifeline

People who have suffered racial, ethnic or cultural genocide, dislocation, displacement, forced immigration, discrimination, persecution and been targeted or disadvantaged en masse to connect with their cultural practices, rituals and ideologies who have posttraumatic mental health problems can benefit greatly by help-seeking from mental health services that are trauma-informed and take into account cultural backgrounds and languages.

In summary, the MindSkiller system 10 enables users to be introduced to all the modern mental health models and practices known to be useful to mental health care at both healthcare system and individual levels, and to encourage their learning about, trailing and utilizing the models that are useful to them anytime that a need arises to help-seek during their journey through the program.

The system 10 of the present disclosure is adapted to assist understanding of the six key categories of mental disorder (Anxiety, Mood, Personality, Psychotic, Substance Use and Eating Disorders) and how they relate to one another. Often sufferers have multiple categories that need to be looked at holistically, how they interact, not in isolation.

The system 10 may facilitate better assessment and treatment coordination, connecting the patient to all the key relevant help-providers to optimize care. It will facilitate general practitioner care, who continues to guide and advise the patient, but now the patient has improved self-understanding and will be a more capable collaborator in their own care. The system 10 may deliver this experience so that patients can access the right information and assistance but keep their healthcare costs down. By using the system 10, users will be provided with the confidence to do their initial research and understand more about their mental health and to have the mental health literacy to communicate about their mental health, whilst they may be waiting for in-person providers to become available.

In another embodiment, where countries, like Australia, have Medicare subsidies, rebates and safety nets available to provision of online mental health care, MindSkiller system 10 will be built to utilize those funding opportunities that encourage care and remove barriers to care. It may deliver a practical and personalized online experience, with some offline, in-person, or by telehealth consultation when needed.

In another embodiment, the system 10 can improve co-ordination, triage and assist the user to become aware of the treatment options based on the disorders and nature of symptoms they are suffering, assisting them in their discussions with help-providers to ask questions and personalize their management package.

In another embodiment, triage platforms may reference the MindSkiller system 10 on their sites showcasing that it offers high-quality mental health content and services—which makes the two approaches complimentary.

In another embodiment, the Mindskiller system 10 of the present disclosure can introduce innovative elements to improve care coordination and triage for use in large corporations and the health funder industry.

1. The capacity to develop an industry-specific version within PLUS services;

2. design and structure of the system 10 permits integration of infrastructural ingredients that enable the development of a cohesive, seamless purpose-specific mental health system; and

3. The categorization mental health system.

The innovative elements of the present disclosure can assist to rein in costs within the health insurance industry in a way that can actually enhance the healthcare and improve health outcomes of the members. This is, of course, dependent on the innovative design, elements and structure of the system 10 of the present disclosure.

In one embodiment, the system 10 is adapted to provide independent outsourced support to healthcare funders (any health insurer or organization employing staff for whom they may find themselves in the position of needing to fund the consequences of poor mental health e.g., sick leave, poor performance, worker's compensation claims) interested in subscribing to a digital mental health platform that provides 3 digital mental health services that have been specifically designed/tailored to effectively, efficiently and ethically provide clinical support to their high-cost members—noting the subgroups that are likely to comprise the members with mental disorders:
members or employees who have developed chronic mental health problems causing unremitting disability and have ongoing and potentially indefinite healthcare requests and welfare support. Some of these have valid treatment-resistant conditions that require assertive interventions to optimize their level of function—which they have not yet received. Some have been classified as treatment-resistant but actually may have been prematurely considered to have poor prognosis and actually with some high quality assessments and interventions may be able to remit or gain much higher levels of function that was recognized as possible.
members or employees with certain types of personality disorders that contribute substantially to the exaggeration and perpetuation of their level of disability fueling healthcare costs and welfare support. Many of these have pre-existing learnt helplessness schemas—poor decision-making capacity to achieve adaptive life goals and poor coping skills leading them to develop maladaptive/unhelpful belief systems and habits that result in their developing learnt helplessness, which fuels their long-term health problems and the high costs of supporting them (these people have what are technically termed ‘internalizing’ maladaptive behaviors or ways of coping with stress or unmet needs—at the expense of their health and wellbeing so making them more dependent and needy on others).
members or employees who are either exploiting the system by exaggerating their disability in order to increase claims or have anger problems, feel entitled to high levels of ongoing care and maliciously demand to be looked after by what they perceive to be a hostile world that owes them care. Most of these have certain types of personality disorders leading them to unhelpful belief schemas driving them to find short-cuts to meet their needs rather than have the maturity to achieve their goals without it being at someone else's expense (these are technically termed ‘externalizing’ maladaptive behaviors or ways of coping with stress or unmet needs where they have disregard for others).

Part of the problem is that the prior art system enables their learnt helplessness because it fails to deal with their underlying problem: that they get secondary gain from being chronically ill by reinforcing the message that they need to depend on their external personal supports, healthcare and allied healthcare providers and other non-healthcare qualified help-providers (all or some of whom may or may not be supplying them with high quality care or the type of care that will enable them to recover or gain optimal functional independence) and the system to financially support their healthcare and wellbeing. It may actually potentially be reinforcing their schemas that they are helpless, untreatable, needy victims of their poor health and inability to function, study or work in any meaningful way.

The prior art system also contributes to their chronicity by responding in a haphazard, inconsistent and bureaucratic way to them and their problems. For example, often a case manager is allocated who tries to collaborate with the client in their coordination and funding of their care. Often it is not a pleasant role as conflicts and disagreements arise when the case manager attempts to rationalize their healthcare choices and management plan. Often the clients perceive the case manager to be an adversary who is preventing them from receiving what they perceive to be in their interests. The help-providers of the client may side with the client and the case manager's level of influence is diminished. Many case managers stay for short periods of time in these roles as they are unpleasant. That leads to some chronic clients having multiple case managers who continue the plan in place as there is resistance from the client or their help-providers to make any changes and arouse the frustration and discontent of the client and their supports. The client's position is thereby reinforced and they become more confident at resisting change that may provide benefit and potential for improved outcomes. Many consumers and their supports are wary and distrustful of any psychosocial support or rehabilitation programs that the funding organization is implementing in-house because they perceive or experience those programs as not being independent and having a biased agenda to reduce their claims and costs rather than truly assist in their healthcare. Hence, the consumers may ‘dig in’ and become defensive of their position and perception that they are chronically ill, disabled and dependent. This describes a ‘vicious circle’ where their helplessness is reinforced as the system is not addressing their underlying problem—which is actually their learnt helplessness and resistance to change even when that change may be for the better. It is possible that those members or employees may not actually wish to get better—they may take solace and derive perverse gratification from being chronically ill and dependent. It is a regression to a childlike state of underdeveloped maturity, incompetence dealing with the outside world and a state of permanent vulnerability where they desire that their supports (external mentors, clinicians and coaches) reinforce that illness-disability unhealthy overdependent state. As time goes by, it no longer becomes a ‘state’ but their ‘trait’—it becomes their new identity: with their internal narrative or schema being that they are a chronically helpless, dependent person who is hopelessly untreatable. They cling to that ‘depressive’ identity and resist maturation of it because the environment is an inherently bureaucratic one that they resist and rebel against in an infantile manner and which ultimately often permits them to reinforce it because their external clinicians who have adopted the helplessness schema of the consumer during their dealings with the consumer and they helplessly ‘tick the boxes’ saying the consumer is permanently psychologically disabled and needs perpetual care and to be taken care of financially for their wellbeing.

The funding organizations tend to respond:
bureaucratically—by looking for loopholes to exclude them;
bureaucratically—by making their life so difficult with all the paperwork and checking and cross-checking and obstacles that they give up ‘fighting’ for more healthcare; or
bureaucratically—by getting multiple medical second opinions to question the validity of their health, their claims and their neediness and getting private investigators to question their level of disability.

Sometimes these responses work but they are tedious, expensive, can damage organizational reputation, reinforce a defensive bureaucratic system culture and methodology, they don't work in many instances—in fact, they may reinforce the problem (as explained above) and they do not address the underlying causative problems.

In another embodiment, the system 10 is adapted to target the needs of the following three groups—those who have had sub-optimal care, those who have ‘internalizing’ personality disorders—have learnt helplessness and dependency, and those who have ‘externalizing’ personality disorders—the exploiters and entitled demanders with anger problems, to improve their health outcomes, to improve their ability to function, to weed out exploiters and those with unreasonable expectations to improve the sophistication of the system so that it is not—unintentionally and expensively—reinforcing chronicity and associated high healthcare costs.

In this embodiment, the system 10 has the capacity to provide an approach where a corporation can outsource the problem members or employees to a PLUS services healthcare provider who has the capacity to—independently, collaboratively and aligned with public healthcare values—provide a high-quality, specialist psychiatry-grade digital, telehealth and virtual mental health service that effectively, efficiently and ethically can add value by providing adjunctive, supplementary and complementary ‘clinical support services’ to the existing healthcare of these groups by their external clinical, allied health and adjunctive healthcare providers. The added digital, telehealth and virtual services will enhance their healthcare, level of function and reduce their criteria as being chronically disabled and thereby lead to reducing overall costs to align with the actual minimal healthcare needs of these groups.

The PLUS healthcare service will have these innovative features that are generally lacking from existing external private and public healthcare systems and digital health platforms:
A digital eLEARNING education and training course that serves as a blueprint for the consumers and help-providers to advance their understanding of their mental health, mental disorders and the types of help-providers and types of interventions that they may provide the consumer for their particular mental health deficiencies and disorders that incorporates all the contemporary and progressive models of care, models of recovery, system models and ethical healthcare values and principles. The program has been built on autonomous learning principles to optimize engagement, adherence and gaining value from the content. The content has been developed to fill gaps in mental health literacy education and builds on and expands the concept of mental health literacy to include not only self-help, but also learning how to coordinate one's own care by finding help-providers that are fit-for-the-purpose of optimally assisting the consumer with their recovery, resilience and mental fitness trajectories—this is consistent with person-centered care and putting the consumer in the center of the system (or building the system around the consumer). The content uses a true ‘visual learning’ methodology and has categorizations that have been painstakingly developed over many years in collaboration with consumers to improve the understanding of the many concepts involved in mental health, mental disorder and recovery minimizing the use of medical jargon, as well as developing skills and strategies to mature mental faculties involved in decision-making and coping with stress and to accelerate the maturation process of the user whilst they are undertaking the course as it has been developed to optimize their participation using some novel devices (it is designed to be ‘infotaining’ through the use of animation and gamification elements, it uses role-playing fantasy with one of the characters being the alter ego of the consumer and the other two are a psychiatrist avatar and a help-provider assistant avatar—so the program is conversational, the user makes choices, there are embedded quizzes, it produces personalized posters based on self-reflective questions, the priority is to make it pragmatic and interesting and to maximize self-reflection). The consumer is able to oscillate between learning about mental health principles in general (the so-called transdiagnostic content that has divided or categorized mental health into ‘areas of interest’ to the consumer) and learning about any specific mental disorders that are relevant to them.
A telehealth CONNECT service that enables accredited providers to provide brief episodes of clinical support that supplement and complement any existing care, support and interventions of external providers, between consultations or during consultations with their providers, and also which may assist the consumer to identify, refine and seek out external providers that fit their needs, in collaboration with their existing general practitioners. The service will enable external healthcare providers to receive progress reports about what services the consumer has engaged in during their virtual mental health journeys using the platform. It will also serve to provide some education to the external providers in the process. Some of the external providers may be enticed to themselves undertake the training in order to obtain certification and accreditation to then serve as online providers to their consumers and others undertaking the eLEARNING courses.
A ‘virtual hospital and community mental health care system’ structure to provide virtual ‘clinical care pathways’ for the consumers and providers that in many ways parallels a bricks-and-mortar hospital's infrastructure, processes and clinical care pathways but has been adopted for the virtual environment online space and been modified to enhance care by incorporating ingredients that promise to improve care.

The purpose-built industry-specific PLUS service will include a specific training program to accredit the help-providers who may be determined to be useful for the consumer to acclimatize the consumer to the external mental health system, types of help-providers and the interventions available to them (by a sole mentor, clinician and coach provider and also by mental health service organizations who will also be accredited to be of a quality that aligns with the healthcare values, principles and models used within the program). The clinical care pathway will be bespoke fit-for-purpose to take into account the specific external healthcare funder environment, context and aims.

It is anticipated that the organizational ‘case manager’ will serve as a ‘mentor’ to the consumer—this will be anticipated to turnaround the unhelpful culture of the case manager being perceived as an adversary or funding blocker.

There will be regular case conferences collaboratively chaired by the case manager and specialist psychiatrist—which the external healthcare providers will be invited into and be required to participate in in order for them to be paid by the funder as per the consumer's contract—to develop a management plan that will include the expected minimal usage of the digital eLEARNING course and the telehealth clinical support.

The management plan will outline the progress, tasks and goals for the user and what they will be required to undertake as there will be minimum expectations of their participation in their self-improvement, recovery and improved functional capacity. That will include minimal amounts of telehealth time with specified help-providers (mentors, clinicians and coaches). The consumer's commitment to the management plan will be monitored by the consumer and all help-providers to ensure that the consumer will adhere to the minimal participation that will be required by their contract—they undertake self-help, that they learn how to help-seek and that they ultimately will help-provide as a peer support to others.

The modified clinical pathway will work most effectively if it will be backed up by contractual clauses that specify that the consumer will need to also take some responsibility for their mental health, well-being and capacity for functional recovery. It will encourage their motivation and participation in the program. The detail of the contract could include financial levers for both the consumer and their external healthcare providers. There could also be milestones that recognize the progress of the consumer in improving their mental health literacy in all levels, as: self-helper, help-seeker and help-provider. When they reach capacity for help-provision they will be effectively be developing a new skillset, which they can then use to become a professional peer support when they graduate from this bespoke version of the mental health literacy platform to the general one available outside of the industry-specific version.

The system 10 of the present disclosure can therefore provide a general-purpose, non-industry specific digital mental health platform containing the three digital mental health services that provide digital, telehealth and virtual clinical support will provide credibility, support the perception of the industry-specific versions being different versions but retaining the independence of the broader purpose product, and will provide an avenue for the consumer to graduate to where they can return to being identified as a non-ill person rather than someone unendingly dependent on others for their wellbeing (when appropriate).

It is innovative to build a modified virtual hospital and community mental health care infrastructure/telehealth service ‘on the footings of’ a digital eLearning education and training program that is designed using new, improved-upon conceptual models of mental health, disorder and mental health care that serve as overarching blueprints that can enhance the care of consumers, the treatment of providers and integration of all infrastructural elements into a cohesive mental health system that has not been possible without the new models and this holistic conceptualization.

In one aspect of the present disclosure, the system 10 comprises an AI engine to process user data and make recommendation(s) to user.

The AI engine may play a large role in MindSkiller system in one embodiment. The AI engine of the system 10 is supported by extensive user data and probably third party (e.g., Facebook, National Health data, OpenGov etc.) data to achieve the “Big Data” status to be able to deliver an AI solution.

In one preferred embodiment, the MindSkiller platform of the system 10 is adapted to capture real time data in Step 101 and ultimately this can feed into the AI engine through Steps 103 to 105 for processing and drive a number of applications in Step 106 that provide a 1:1 personalized service through MS, including (but not limited to):
1. Recommendations on eLEARNING service based on their profile
2. Personalized e-learning based on the individual's profile and behavior—including VR/XR
3. Automated assistance for users online (Bots) and with their training
4. Journey assistance to the eLEARNING (helping users navigate through the platform based on their profile)—front end management
5. Matching the right Help-providers with Help Seekers; and
6. AI assisted, predictive Diagnosis on their mental health disorders, based on the data they and others have shared with MindSkiller.

In one embodiment the AI engine 100 of the system 10 is adapted to provide recommendations on eLEARNING service based on their profile.

The AI engine is adapted to aid data-analysis of user profiles (disorder categories and areas of interest, help provider choices—past, existing and future, contents pages) which could help to personalize/individualize the recommendations of what order to undertake the eLEARNING program and use of the other two packaged digital services such as CONNECT and PLUS.

The eLEARNING Sytem 132 is adapted to provide questions and receive feedback from user to supplement and enhance with three types of mental health literacy-improving these 3 coping strategies:
self help
help seeking
help providing

MindSkiller refers to 3 sets of mental skills:
(1) 3 Navigation Tools—attention, perspective, action
(2) 3 Coping Tools—rational, emotional, reflective
(3) 3 mental health literacy acquisition tools or ‘Coping Strategies’—Self Help, Help Seeking, Help Providing. All three sets add up to a user's ‘MindSkills’).

In another embodiment, the AI engine 100 is adapted to provide personalized e-learning based on the individual's profile and behavior—including VR/XR. The AI engine 100 is adapted to aid data-analysis of the user's choices made within the 3 services to enhance how they access and journey through the eLEARNING service, CONNECT and PLUS, to improve, refine and maximize their mental health literacy skills and strategies. This may also drive recommendations for (or, if user chooses, automatic access to) CONNECT and PLUS add-on services.

The AI engine 100 may provide automated assistance for users online (Bots) and with their training in a preferred embodiment. Various learning AI options as listed below may be provided.
AI versions of both Dr Egg and the Tug assistant could be available (instead of the pre-programmed predetermine routine for every user).
If AI is turned on for Dr Egg or Tug, then the user could choose any proportion from the 3 types of therapies for them: Cognitive-behavior therapy-based (CBT), Mindfulness-based therapy (MBT) and Reflection-based therapies (RBT).
The user could, for example, choose: Dr Egg to be 80% CBT, 10% MBT and 10% RBT and Tug to be 100% CBT (no MBT or SBT)—either avatar's AI could be allocated any % of each therapy type.
AI engine 100 is adapted to communicate with the nominated external health/help providers (assessment and progress reports, risk and warning indicators, intervention indicators, even diagnostic indicators).
Temporal analyses to determine changes in user mental states e.g., relapse, increased suicide risk—inhouse (MindSkiller and partner ecosystem) and external (PLUS—Risk Monitor program—general use)
Research tool to guide future research questions due to data provision and pattern analysis

The AI Engine 100 in one embodiment may provide journey assistance to the eLEARNING (helping users navigate through the platform based on their profile)—front end management. The AI engine 100 may aid data-analysis of the user's choices made within the 3 services to enhance how they access and journey through the eLEARNING service, CONNECT and PLUS, to improve, refine and maximize their mental health literacy. This may also drive recommendations for (or, if user chooses, automatic access to) CONNECT and PLUS add-on services.

The AI engine 100 in one preferred embodiment is adapted to conduct matching for the right Help providers with Help Seekers. The AI engine 100 may aid matching of the Help seeker with Help providers include:
Individual Help providers from the 3 categories
Help providing digital mental health services

Regarding the services, this includes:
Industry specific groups—healthcare industry
   i. Private hospital-based services—inpatient, day patient, in-home care, digital/virtual care
   ii. Public health sector—hospital, community center, digital/virtual care
   iii. Private health Insurer
   iv. Workers' compensation insurer
   v. Income protection/salary continuance insurer
Virtual reality digital mental health services for the 3 types of therapies: CBT, MBT, RBT
Learning AI digital services for the 3 types of therapies: CBT, MBT, RBT
Remote neurostimulation digital connection services—TMS, tDCS
Access to other innovative emerging interventions (including via research trials and associated private clinics e.g., Black Dog Institute).

In one embodiment, the AI engine 100 is adapted to assist, predict diagnosis on mental health disorders, based on the data of the user and others have shared with MindSkiller. In a preferred embodiment, this is represented by ‘diagnostic meters’ into the sails where there is a percentage probability of this being a ‘disorder/diagnosis of interest’ based on the user's preferences and choices and interactions with the platform. This can be divided into either being a ‘diagnosis of interest’ or a ‘diagnosis’ based on the type of user (self-helping or helping others). In one embodiment, the user may engage in eLEARNING provided by the system 10. The user interacts with eLEARNING service that may contain a series of story narratives, questions and answers. The system 10 will collect the input of the user. In a preferred embodiment, the system 10 comprises video and audio input and output devices such that the image and sound of the user may be collected as well. The AI engine 100 may under transdermal optical imaging or voice recognition to detect underlining moods or conditions of the user when engaging with the eLearning system. In order to do so, the inputs of the user will be tagged with the user ID and timestamp. Other more invasive equipment, such as EEG, ECG, BPM, devices may be connected to the user device for data collection as well. In this way, a deeper meaning and understanding of the user interaction can be analyzed to provide a better personalized program.

In one embodiment, the system 10 comprises a grid:
Disorder Categories (6 banners)
Disorder mechanisms (3 crows nests/sails)
Diagnoses (over 40 diagnostic stripes)

The grid reflects the 3 steps that supplement and enhance traditional classification:

Step 1: The ‘Categories of Mental Disorder’ are simplified from existing classification systems into 6: Mood, Anxiety, Personality, Psychotic, Substance use and Eating Disorders.

Step 2: All causative ‘mechanisms’ are divided into 3 general subtypes—‘Brain Body and Mind.’

Step 3: A specific psychiatric ‘Diagnosis’ given to an individual represents the nicknames used by psychiatrists that is the product of the marriage between the ‘category’ of disorder (the formal descriptive classification) and one of the ‘subtypes’ (underlying general causative mechanism).

The AI engine 100 is adapted to add valance to each of these steps. Like a ‘barometer’ next to each of the elements of the grid changing/being revised as the user uses the platform.

In one embodiment, the system 10 comprises an AI Engine 100 adapted to provide ‘Help Seeking’ strategies from the 3 kinds of Help Providers. This is particularly helpful for someone who has reached the limits of their abilities to ‘help themselves.’ These three kinds of Help Providers are:

Clinicians with clinical expertise such as Psychiatrists, General Practitioners, Mental Health Nurses, Psychologists, Psychoanalysts, Social Workers, Occupational Therapists and Dieticians

Mentors with lived, personal or life experience such as Peer supports, Carers, Family Members and Friends

Coaches with specific well-being and performance-enhancing training methods such as Personal Trainers, Counsellors and Case Managers

The AI engine 100 is adapted to recommend types of clinical supports to the user whilst they are undertaking the education and training in eLEARNING—to sample the different types of Help-Providers via the CONNECT service, which will guide users about what each of the Providers has to offer. Users will thereby gain a deeper understanding of the types of support and interventions available.

Whilst mental health literacy traditionally consisted of understanding about mental disorders, their management and the principles of behavioral and biological interventions, MindSkiller system 10 may help a user to learn how to:
‘Help-Seek’ (so the ‘CONNECT’ service assists users to experience/sample real-life trained/accredited Mentors, Clinicians and Coaches);
Learn how to put together a ‘management plan’ (each disorder module has a grid that personalizes the salient aspects of that diagnosis for the user and it concludes with 3 posters derived from the user's choices/interests/matched recommendations and a links section that matches their choices to individual providers/service providers/medical information that are on the database from CONNECT, PLUS and any other information database tailored to suitable for the user in a virtual hospital provide by the system 10 of the present disclosure); and
Navigate the health system (the CONNECT service will assist users to access information about their local health system from real-life trained/accredited Mentors, Clinicians and Coaches who have been selected by the user based on characteristics of interest to them. The AI engine 100 may match help-providers who will be able to answer the questions of interest to that individual user. The AI engine 100 is adapted to also be used for this aspect of mental health literacy to help the user with triage).

An example of the disorder module (termed ‘Diagnosis Subunit’) of the system 10 of the present disclosure comprises the following:
SUBUNIT CATEGORIES:
1. TRANSDIAGNOSTIC UNITS/SUBUNITS
2. KNOWLEDGE CHECK SUBUNITS
3. DIAGNOSIS SUBUNITS
UNIT 1 MAPPING THE JOURNEY TO YOUR LIFE GOALS
Subunit 1.1 Getting Your Bearings
link to a storyboard of eLEARNING system (the storyboard is designed by the professional. The storyboard may change in accordance the different scenario)
Knowledge Check 1.1
link to Dashboard of eLEARNING system
Subunit 1.2 In Search of Your Life Goals
link to a storyboard of eLEARNING system
Knowledge Check 1.2
link to Dashboard of eLEARNING system
Subunit 1.3 Introducing the 3 Navigation Tools
Part I Reaching Peak Performance
link to a storyboard of eLEARNING system
Link to Dashboard of eLEARNING system
Part II Staying in the Healthy Range
link to a storyboard of eLEARNING system
link to Dashboard of eLEARNING system
Knowledge Check 1.3
link to Dashboard of eLEARNING system
UNIT 2 COPING WITH EXTERNAL STRESSORS
Subunit 2.1 Introducing the 3 Coping Tools: Rational, Emotional and Wise Minds
link to a storyboard of eLEARNING system
link to Dashboard of eLEARNING system
Knowledge Check 2.1
link to Articulate360:
Subunit 2.2 A Brief Tour of the Rational Mind
link to a storyboard of eLEARNING system
Link to Dashboard of eLEARNING system
Knowledge Check 2.2
link to Dashboard of eLEARNING system
Subunit 2.3 A Brief Tour of the Emotional Mind
link to a storyboard of eLEARNING system
link to Dashboard of eLEARNING system
Part I The Mood Switch
link to a storyboard of eLEARNING system
Link to Dashboard of eLEARNING system
Part II The Reality Compass
link to a storyboard of eLEARNING system
link to Dashboard of eLEARNING system
Knowledge Check 2.3
link to Dashboard of eLEARNING system
UNIT 3 COPING WITH UPS AND DOWNS
Subunit 3.1 A Guide to Classifying Mood and Anxiety Disorders
Part I Descriptive Classifications: The Mood and Anxiety Banners
link to a storyboard of eLEARNING system
link to Dashboard of eLEARNING system
Knowledge Check 3.1 Part I
Link to raw MCQs and Line Matching Questions:
Link to Dashboard of eLEARNING system
Part II Explanatory Classification: Mind, Body and Brain
link to a storyboard of eLEARNING system
link to Dashboard of eLEARNING system
Knowledge Check 3.1 Part II
Link to raw MCQs and Line Matching Questions:
Link to Dashboard of eLEARNING system
Subunit 3.2 A brief tour of Mood Disorders: The Mood Stripes
Link to Storyboard
Link to Dashboard of eLEARNING system
Knowledge Check 3.2
Link to raw MCQs and Line Matching Questions:
Link to Dashboard of eLEARNING system
Diagnosis Subunit Banner: Mood Sail: Mind Stripe: Reactive Depression
link to a storyboard of eLEARNING system
link to Dashboard of eLEARNING system
Diagnosis Subunit Banner: Mood Sail: Body Stripe: Mixed Anxiety-Depression
link to a storyboard of eLEARNING system
link to Dashboard of eLEARNING system
Diagnosis Subunit Banner: Mood Sail: Brain Stripe: Melancholic Depression
link to a storyboard of eLEARNING system
link to Dashboard of eLEARNING system
Subunit 3.3 A brief tour of Anxiety Disorders: The Anxiety Stripes
link to a storyboard of eLEARNING system
link to Dashboard of eLEARNING system
Knowledge Check 3.3
link to raw MCQs and Line Matching Questions:
link to Dashboard of eLEARNING system
Diagnosis Subunit Banner: Anxiety Sail: Mind Stripe: PTSD-Acute Stress Disorder
link to a storyboard of eLEARNING system
link to Dashboard of eLEARNING system
Diagnosis Subunit Banner: Anxiety Sail: Brain Stripe: Obsessive Compulsive Disorder
link to a storyboard of eLEARNING system
link to Dashboard of eLEARNING system
UNIT 4 COPING WITH INTERNAL STRESSORS
Subunit 4.1 A brief tour of self-sabotaging scripts
Link to Storyboard:
Knowledge Check 4.1
link to raw MCQs and Line Matching Questions:
link to Dashboard of eLEARNING system
Subunit 4.2 A guide to classifying personality disorders
Part I Descriptive Classifications: The personality Banner
Part II Explanatory Classification: Mind, Body and Brain
Subunit 4.3 A brief tour of Personality Disorders: The Personality stripes
link to Storyboard:
link to Dashboard of eLEARNING system
Knowledge Check 4.3
link to Dashboard of eLEARNING system
Diagnosis Subunit Banner: Personality Sail: Mind Stripe: Borderline Personality Disorder
link to Storyboard:
link to Dashboard of eLEARNING system
Diagnosis Subunit Banner: Personality Sail: Body Stripe: Functional Neurological Disorder
link to Storyboard:
link to Dashboard of eLEARNING system
Diagnosis Subunit Banner: Personality Sail: Brain Stripe: Attention Deficit Hyperactivity Disorder
link to Storyboard:
link to Dashboard of eLEARNING system
UNIT 5 COPING WITH ‘BEING OT OF TOUCH’ WITH REALITY
Subunit 5.1 A Brief Tour of Distortion and Denial of Reality
link to Storyboard:
link to Dashboard of eLEARNING system
Knowledge Check 5.1
link to raw MCQs and Line Matching Questions:
link to Dashboard of eLEARNING system
Subunit 5.2 A guide to classifying Psychotic Disorders
Part I Descriptive Classifications: The Psychosis Banner
Part II Explanatory Classification: Mind, Body and Brain
Subunit 5.3 A Brief Tour of Psychotic Disorders: The Psychosis Stripes
Knowledge Check 5.3
Diagnosis Subunit Banner: Psychosis Sail: Mind Stripe: Brief Reactive Psychosis
link to Storyboard
link to Dashboard of eLEARNING system
Diagnosis Subunit Banner: Psychosis Sail: Body Stripe: Delusional Disorders-Body Dysmorphia, Parasitosis and Delusional Jealousy
link to Storyboard:
link to Dashboard of eLEARNING system
Diagnosis Subunit Banner: Psychosis Sail: Brain Stripe: Schizophrenia and Schizoaffective Disorders
link to Storyboard
link to Dashboard of eLEARNING system
UNIT 6 COPING WITH FIXATIONS
Subunit 6.1 A guide to classifying Substance Use and Eating Disorders
Part I Descriptive Classifications: The Substance Use and Eating Disorder Banners
Part II Explanatory Classification: Mind, Body and Brain
Subunit 6.2 A Brief Tour of Substance Use Disorders
Knowledge Check 6.2
Diagnosis Subunit Banner: Substance use Sail: Mind Stripe: Substance Abuse
Link to Storyboard:
Link to Dashboard of eLEARNING system
Diagnosis Subunit Banner: Substance use Sail: Body Stripe: Substance Dependence—Psychological
Link to Storyboard:
Link to Dashboard of eLEARNING system
Diagnosis Subunit Banner: Substance use Sail: Brain Stripe: Substance Dependence—Physical
Link to Storyboard:
Link to Dashboard of eLEARNING system
Subunit 6.3 A brief tour of Eating Disorders
Knowledge Check 6.3
Diagnosis Subunit Banner: Eating Sail: Mind Stripe: Bulimia
Link to Storyboard:
Link to Dashboard of eLEARNING system
Diagnosis Subunit Banner: Eating Sail: Mind Stripe: Obsessive-Compulsive Eating Disorder
Link to Storyboard:
Link to Dashboard of eLEARNING system
Diagnosis Subunit Banner: Eating Sail: Mind Stripe: Anorexia Nervosa
Link to Storyboard:
Link Articulate360:

The 3 types of intervention from ‘Help Seeking’:
Medical treatments
Psychological therapies
Positive lifestyle choices

These interventions can serve as a guide about what the possible ‘ingredients’ of a personalized ‘management package’ might look like:
My ‘Management Plan’(Poster 3)
The Medical Treatments I am trying are:
1^{st} line______
2^{nd} line______
3^{rd} line______
The Psychological Interventions I am using are:
CBT______
Mindfulness______
Schema______
The Lifestyle Factors
I am using are: Exercise______
Diet______
Social activities______

An example from the ‘Reactive Depression’ module (Diagnosis Subunit) is shown below.
Positive lifestyle choices for Reactive Depression
Exercise routine:
Personal trainer
Fitness center, club or gym
Exercise Physiologist
Yoga or Pilates instructor/classes
Sports clubs, running clubs, cycling club, bootcamp, martial arts, events (marathons, triathlons, charity runs)
Wellness and fitness Apps
Therapeutic massage, spas and beauty salons
Diet:
Dietician
Nutritionist, Chinese medicine and other alternative therapists
Healthy eating and low calorie meals, weight loss clubs and counselling services
Self-help groups
Wellness and fitness Apps
Social activity:
Meetup groups, events and clubs, including religious, cultural and ethnic based
Hobby based activities—individual and group
Volunteering/community service/charitable events
Relationship, dating, introduction and matchmaking Apps and events
Study based activities—professional development and personal interests
Professional healthcare consultations
Psychological Therapies for Reactive Depression
First-line:
Psychoeducation [POPUP: Understanding the nature of the disorder and rationale for a management plan and treatments]
Supportive [POPUP: Encourage healthy adaptive coping]
Interpersonal therapy [POPUP: Dealing with life events and relationship problems (usually 12-16 weeks]
CBT [POPUP: Challenge and change unhelpful thinking patterns, improve coping by problem solving/graduated exposure plan, behavior activation]
Additional therapies:
Mindfulness based therapy [POPUP: Observe thoughts (reflecting rather than reacting) and meditation]
Insight-oriented therapy [POPUP: Gaining understanding by uncovering unhelpful ideas and relationship patterns]
If first-line therapies are ineffective:
Schema therapy [POPUP: Gaining understanding by uncovering self-sabotaging narratives/storylines]
Acceptance & commitment therapy [POPUP: Mixes mindfulness & behavior change strategies]
Psychoanalytic therapy [POPUP: Gaining understanding by uncovering unconscious defenses and conflicts utilizing the therapeutic relationship itself (usually 2+ years)]
Medical Treatments for Reactive Depression
First-line:
No medical treatment
Additional treatments:
Benzodiazepines [POPUP: anxiolytics for daytime panic attacks and sleeping tablets for insomnia]
B-Blockers [POPUP: propranolol or pindolol for physical distress]
Low-dose antipsychotics [POPUP: for night-time agitation (e.g., quetiapine)]
If first-line treatments aren't effective:
SSRIs or SNRIs [POPUP: Selective serotonin reuptake inhibitors and Selective noradrenaline reuptake inhibitors are deactivating or activating antidepressants]
TMS [POPUP: Transmagnetic stimulation if med regime unchanged for 3-weeks]
Mood stabilizers [POPUP: lithium, carbamazepine or valproate if ‘masked’ bipolar disorder (esp. bipolar disorder type 1) a possibility]
Lamotrigine [POPUP: if ‘masked’ bipolar depression (esp. bipolar disorder type 2) a possibility]
Older antidepressants TCAs, MAOIs [POPUP: Tricyclic antidepressants and monoamine oxidase inhibitors]
ECT [POPUP: Electroconvulsive therapy course of 12 if ‘masked’ melancholic depression a possibility]
tDCS [POPUP: Transcranial Direct Current Stimulation]
Stimulants [POPUP: dexamphetamine, methylphenidate or atomoxetine if ‘masked’ attention deficit and/or chronic fatigue disorder

The system 10 may also provide examples of the positive lifestyle choices those with a dominant emotional mind:
If you have a dominant Emotional Mind, I suggest:
You have an exercise routine of cardio and muscle-strength training that is flexible, fun and sociable. Make sure your Sleep-Wake Cycle is as regular as possible.
Your Diet is low in sugars and fats, moderate in protein and preferencing high quality carbs. Aim for 3-5 small meals per day and personalize it based on activity levels, body weight and individual goals. Try to stay consistent with your meal plan 80% of the time and allow rewards.
Your Social activity includes regular contact with family and friends, and some attendance at interest groups and charitable, religious and cultural events. With any professional support, maintain attendance as required.

The AI engine 10 can help refine, improve and enhance the matching of services and information for the user in their personalized ‘My Management Links to Help-Providers, Services and Information webpage at the end of each disorder module.

In another aspect of the present disclosure, there is provided a Mental Healthcare System optimization through access to a specialist-grade ‘virtual mental health system infrastructure.’

The system 10 can enhance product validity of the PLUS additional services due to it being seen to be and experienced as an adequately independent healthcare service.

The present disclosure of the ‘virtual mental health system infrastructure’ can effectively supplement and complement existing mental health care in the treatment of mild, moderate and severe mental disorders could enhance the:

(a) management of mental disorders, poor mental resilience and low mental fitness;

(b) quality of care (improving effectiveness by enhancing the patient's ability to understand their problems, take more responsibility to coordinate their care and to help seek)

(c) measurable health and satisfaction outcomes

(d) efficiency of care (in health economics terms ‘getting more bang for your buck’ by improving overall ‘system productivity’ levels, at the individual level reducing ‘loss of productivity’ and at the funding level reducing welfare and overall healthcare funding costs).

The system of the present disclosure enables the integration of the digital mental health services, which can seamlessly provide clinical support to primary and specialist clinicians who are desperately needed to enhance standard outpatient and/or inpatient mental healthcare—that is, healthcare delivered face-to-face or by telehealth by any unaffiliated healthcare provider but particularly aimed at clinicians such as general practitioners, psychiatrists and psychologists.

The different between the prior art and the present disclosure is that the system of the present disclosure is not a duplication of existing services—such as another emergency or crisis service, which is only required by a minority of their patients and there are existing online and phone crisis support services available. In fact, the gap could be understood as what the majority of clinicians' patients do not need or want:

(a) another service for crisis support (this space is taken e.g., Lifeline and phone support from multiple other services)

(b) a digital mental health service targeted very narrowly at some specific disorder (mild anxiety and depressive disorders)

(c) a digital mental health service providing a specific narrow treatment modularity such as iCBT (either disorder specific or transdiagnostic iCBT, even if guided by clinicians e.g., MoodSpot)

(d) an overly simplistic iCBT wellbeing course designed by academics rather than clinicians.

Another advantage of the present disclosure is that the system of the present disclosure is able to balance the supply and demand for clinical specialist-grade psychiatric services.

Because it is increasingly difficult to get appointments with private psychiatrists (long waiting lists, poor ability to match the patients with the optimal psychiatrist for their needs and increasing gap payments are making private psychiatrists less and less affordable and accessible) and both public hospital and community mental health services have increasingly raised the entry bar and access to those services are increasingly unavailable to the majority of patients. That's because those services are increasingly being reserved for severe acutely disordered patients with high risk level or behaviors. Public services are increasingly catering to crisis care and people with severe chronic mental illnesses e.g., schizophrenia complicated by substance abuse, where they are a danger to themselves or others and need to be maintained on depot antipsychotic medications.

That's part of the reason referral to psychologists has become so prevalent by general practitioners—so they can access psychological care for their patients much faster and more easily. In fact, many psychologists have rented rooms within general practitioner practices since the introduction of the Medicare Better Access Scheme in Australia that subsidizes limited numbers of psychology consultations.

Despite this increasing gap and delay between supply and demand in being able to access specialist-grade psychiatric services, general practitioners, psychiatrists, psychologists and other clinicians have not been referring as a standard practice their patients to digital mental health services to fill the gap because these are not specialist-grade services. They are much more limited, narrower, oversimplistic and lack the ingredients that are needed to provide an effective, quality, efficient and user-friendly service to supplement and complement primary care—or to make specialist care more efficient (increased throughput). What would be particularly appreciated would be services that can provide input that would assist the general practitioners and psychologists to help support their patients whilst they are waiting for access to the specialist psychiatry and specialist health services.

If a digital mental health services can speed up recovery times that would make the process more productive and more efficient by leading to more patients being seen in the same amount of time by the specialists. Only a specialist-grade digital mental health services can have this capacity because that is the expert knowledge, skills and strategies the primary care providers and the consumers optimally require for more severe conditions that are not self-limiting. An effective digital mental health services would enable this rare and valuable expertise (currently only available by a small group—there are only 3,500 psychiatrists throughout Australia) to be provided at scale.

This gap is also there for people without primary care clinicians who want access to digital mental health services of high quality who are seeking education about mental health before seeking help from anyone. They want to improve their mental health literacy. They may not know who to see. They may not have a regular general practitioner or not wish to speak to their general practitioner without doing their own research first. Most people want to go to the internet first, especially if they perceive their health problem to be personally sensitive or embarrassing due to their subpopulation culture. In some countries, general practitioners are not the coordinator of peoples' health or mental health. In those cultures, people prefer to go directly to specialists.

It has been identified, through extensive research and development, that part of the reason access to digital educational support and guidance mental health services has not been effectively done to date is that it has not been recognized that such a virtual system infrastructure needs to be constructed of equivalent parts to that of an external bricks and mortar mental healthcare system's healthcare facilities, the multidisciplinary team (specialist clinicians, trainee doctors, junior doctors, allied health clinicians, other support staff), specialist-designed clinical tools, contemporary healthcare culture and practices, capacity to accommodate and facilitate academics to conduct research, research translation and innovation, processes to ensure minimum standards with regulations, accreditation processes, clinical care pathways and healthcare values and ethical practices.

The present disclosure is able to solve the above problem by providing a highly sophisticated clinical enhancement tool in the management of mental disorders that has the following structural elements:

(1) A method of teaching that makes the educational and training content applicable to the full spectrum of users, such as referred patients; independent consumers wanting to “help themselves” become more mental health literate; and providers such as: mentors (includes peer supports, family, carers, educators and organizational case managers), clinicians (medical clinicians include general practitioners and psychiatrists, non-medical include psychologists, mental health nurses, social workers, dieticians and occupational therapists), and coaches (such as personal trainers, exercise physiologists, certified coaches);

(2) Therapeutic ‘virtual space’ adapted to provide the full stakeholder spectrum of self-help, help-seeker and accredited help-providers to interact within to enhance clinical care (which includes specified limited interactions with external help-providers) that has purpose-built clinical care pathways guiding all the users toward interactions that will achieve the goals of enhancing mental health literacy developed from a synthesis of all the contemporary healthcare models of care, which create a ‘scaffolding’ to (which can retain an adequate and necessary degree of flexibility and choice that will ensure the users can personalize their experiences to refine the type of knowledge acquisition they need to improve their decision making and coping skillsets maturation and achieve optimal health outcomes);

(3) Hospital-grade healthcare standards, regulations, accreditation processes adapted to support the clinical care pathways, which facilitate and maintain quality control for all users (consumers, mentors, clinicians, coaches and even external consumer helpers);

(4) Convertibility that allows the system to convert into bespoke or modified versions for specific industries or purposes to assist to reduce the negative effects of lowered mental health or mental disorder on productivity, sick days and healthcare and welfare costs, in particular, for large corporations and health insurance companies (private health insurers, workers comp insurers and life insurers)

The present disclosure also provides a theme-based universally-applicable ‘symbolic visual language’ and system of explanatory categorizations to enhance a descriptive classificatory system that increasingly neglects explanatory formulations—as a solution to this absence.

With the highly sophisticated clinical enhancement tool in the management of mental disorders discussed above, the system of the present disclosure is particularly prominent in current circumstances of a global pandemic where it benefits all stakeholders to minimize the amount of face-to-face care required. Some products have been developed that are purported to be digital mental health services that are supplementary and complementary to standard care but they are missing components that are necessary to achieve a level of quality that are required to create a functional virtual hospital-community-industry infrastructure that reflects modern clinician (primary and specialist level) and user expectations and capacity.

Another benefit of the present disclosure over the prior art is that the system of the present disclosure is adapted to provide easy-to-digest explanatory categorizations for mental health concepts including the use of a descriptive approach.

A descriptive approach has dominated the past thirty years since the advent of DSM—but had the negative consequence of unintentionally fueling the oversimplification and overgeneralization of diagnoses and as a result, the entire field of mental health, which has increasingly shifted from specialist to primary care. And in primary care, diagnostic formulations, which has been the more sophisticated biopsychosocial explanatory method employed by generalist psychiatrists since the introduction of DSM, has become lost as a method of understanding the patient and contributing toward the management approach/plan. Diagnostic formulation has become less and less employed even at the specialist level. As a result of these developments, the diagnostic reliability has improved but explanatory understanding has waned. This has not occurred by psychiatrists who practice predominantly psychoanalytic psychotherapy, but that group has become less and less prominent and in fact have become a small but significant minority. That group has continued to shrink and is likely to become a small minority in the future, with the biomedical paradigm more and more dominant, which has also encouraged a more descriptive diagnostic approach largely free of aetiological models.

There has been a more generalist approach used to patient care where the type of management given has been less dependent on the faction the treating psychiatrist belongs to—generalist or psychoanalytic practice. In fact, cognitive behavior therapy and mindfulness-based therapies have grown in popularity whilst most generalists are informed in these and sometimes in psychoanalytic or the milder but also insight-oriented psychodynamic psychotherapies. Hence, psychiatry has become more and more a multi-modal, multi-dimensional assessment and management approach.

The rapid flux in the way psychiatry has been practiced over the past seventy years has led to a delay in public knowledge and how well taught and capable primary care clinicians are in the management of mental disorders. Indeed, psychiatrists themselves have been poor educators of non-psychiatrists and even older psychiatrists have struggled to keep up with developments. Cutting-edge multi-modal psychiatry utilizing all the modern models is not ubiquitously practiced.

One advantage of the system of the present disclosure is the provision of psychiatrists developing education and training models fit-for-purpose to explain, teach and train non-psychiatrists. The system of the present disclosure is able to allow adeptly psychiatrists psychoeducate their patients, their families and the public about modern psychiatric knowledge, models of psychopathology and multifaceted management packages.

Evidence of this is that despite millions of taxpayer dollars being spent on public mental health literacy campaigns, the mental health literacy of the community is actually poor if gauged by sophistication of knowledge about mental disorders, recovery and resilience. There are many “R U OK” campaigns, but that is where the knowledgebase seems to abruptly fall away. One of the highest prevalent mental disorders causing morbidity and mortality is major depressive disorder. Many patients with severe depression are shocked by how ignorant their peers, families and even help-providers are about the different types of depression that exist. That is a failure of quality mental health literacy.

The system of the present disclosure is able to provide virtual hospital-community healthcare system models using digital health platforms and telehealth to provide assistance to health funders paying for the healthcare or welfare payments to consumers with mental health problems who are chronically disabled and ill due to specific types of mental disorders where the consumer's insatiable desire and help-seeking is itself unhealthy and part of their mental health problems and funding that maladaptive healthcare or welfare support actually positively reinforces their dependency, helplessness and inappropriate help-seeking behaviors that are due to:

(a) self-sabotaging their health outcomes due to types of personality disorders that lead to various types of harmful behaviors that propagate their symptoms and loss of function and resistance to recovery, which could be categorized as:

(b) externalizing behaviors—where the consumer is benefiting at others' expense

(c) internalizing behaviors—where the consumer has poor coping at their own expense

(d) loss of touch with reality—where the consumer makes consistently irrational decisions and engages in inconsistent behaviors

(e) engaging in therapeutic relationships that are suboptimally assisting them to recover from their disorders, disabilities and in fact may be unintentionally ‘enabling’ their unhealthy/unhelpful dependencies and resistance to recover.

All these consumers are likely to be the highest cost individuals to any healthcare system and healthcare funding or welfare support schemes.

In one embodiment, the present disclosure provides a psychiatry-grade broad scope, pragmatic and clinically-oriented eLearning service for improving mental health outcomes, recovering from mental disorders, reducing psychopathology and improving resilience and mental fitness, that is highly engaging, understandable and utilizable, maintaining user interest to achieve high completion rates, with all types of user needs catered to by the same product:

(a) Consumers with mental health problems (or who are perceived to have problems by their close contacts): those interested in learning about all levels of mental health literacy categorized as self-help, help-seeking and help-providing (as a peer support to a help-seeker engaging in the eLEARNING service, or other types of mentors such as a carer, employer or case manager of a client receiving healthcare funding or support);

(b) Help-providers: mentor, clinician (medical and non-medical) and coach types of help-provider wishing to gain mental health literacy training and accreditation that will:

(i) enable them to provide online ‘clinical support’ to help-seekers using the MindSkiller platform

(ii) assist to upgrade their skillsets in their own external practices and workplaces

(iii) gain CPD points or credits toward their registration bodies' and professional associations' or colleges' CPD programs

In one preferred embodiment, the system of the present disclosure comprises a mental health consumer-provider brokerage service interface that uses clinical care pathways for the education, training and accreditation, and provision of ‘clinical support’ services, which has an underlying virtual hospital infrastructure that ensures minimal standards and quality control, and that utilizes contemporary psychiatric care practice models, principles and healthcare values.

The system of the present disclosure is adapted to provide models of care designed specifically to optimize the technological capacities and potential of the Internet to support an advanced type of mental health literacy through ‘clinical support’ services that can supplement and complement existing mental health care being received to enhance that care by putting the consumer in the center of the system to increase their ownership, responsibility and capacity to serve as a coordinator of their own care.

The present disclosure is able to address problem of the absence in the e-mental health online marketplace of a sophisticated specialist-grade digital mental health literacy education, training and support program for clinicians to refer their mental health patients to or for individuals not receiving any care to access by providing a system adapted to deliver:

(i) Trans-diagnostic—suitable for people with quite different mental disorders or combinations of disorders

(ii) Trans-modal—relevant to users and providers engaged in quite different types of interventions, and

(iii) Trans-cultural or trans-user—truly suitable for different types of users—be they consumers who are interested only in self-help, or are interested in help-seeking assistance from others, or help-providers of different craft groups.

The present disclosure is adapted to provide an e-Learning program. This e-Learning program is adapted to provide the user with the capacity to oscillate between trans-diagnostic and disorder-specific knowledge, skills and strategy.

That is because the current system is adapted to provide contents reflecting an understanding of mental health, mental disorders and psychological distress in terms of practical models with breadth such as the ‘Biopsychosocial Model’ that has become the core explanatory model central to psychiatric practice that informs all assessments, formulations and how treatments and interventions are packaged, prioritized and practiced by psychiatrists and multidisciplinary teams for mental health consumers. In one embodiment, the system comprises an electronic circuit that is adapted to retrieve such contents and deliver to users through a biopsychosocial model interface.

Current online consumer-oriented educative material about mental health is either overly specialized and unfit for most consumers, very narrowly focused (apps) or overgeneralizing, incomplete, irrelevant, misleading or over-simplistic for most individuals because it is almost ubiquitously CBT-based (“cognitive behavior therapy” is a problem-based, solution-focused, reductionistic approach to explaining and managing many types of symptoms and disorders that is almost inane in its simplicity—but it has proven to be a very useful approach. However, it has become recognized increasingly to be inadequate or unhelpful for many individuals. It has been much improved upon by the development of so-called “third-generation CBT” approaches incorporating “mindfulness-based therapies” (MBT)/approaches/principles, but these also are rather simplistic when used in isolation, and poorly taught to patients (and clinicians—for example, most general practitioners would have little capacity to use either CBT or MBT), such that many people still find them lacking or inadequate without other modalities co-utilized—especially medication or “pharmacotherapy”).

Most existing mental health websites have overviews, helper's directories and educational material—but that content is very factual and impractical for what really needs to be done to tackle the disorder.

What is glaringly lacking is online information that is explained adeptly based on a psychiatric lens that combines the explanatory biopsychosocial model (which incorporates CBT and MBT) with the purely descriptive DSM5 that has become the gold standard in classification—but purposefully provides no explanations whatsoever (this ensures reliability of diagnosis—something absent pre-1950s—but does poorly on validity: i.e., how valid is major depressive disorder in two different people with depression? Poor! But that doesn't stop most patients seeing a general practitioner being prescribed an antidepressant, most patients seeing a psychologist receiving CBT, MBT or support and counselling, most patients seeing an analyst analytic therapy; most patients seeing a psychiatrist do end up on a combination of medication and a mix of different types of therapies i.e., a multimodal approach).

In another preferred embodiment of the present disclosure, the system is adapted to provide “Reading Eggs” for mental health literacy. Even when online information has more sophisticated content, it is not easy to understand, or not very palatable or not presented in a straightforward, comfortable, entertaining manner to digest.

Current programs are associated with poor adherence, with high drop-out before completion and are poorly motivating to entice users to keep using the product over longer periods of time or to return repeatedly to it such at times of distress or relapse. That is in direct contrast to face-to-face psychiatric care, which is often associated with good engagement, persistence and return at times of relapse.

Autonomous learning theory postulates that for people to want to keep learning a program needs to be not only useful and interesting, but also engaging. MindSkiller was developed to have all of these ingredients. Much of its core content was developed and tested in the field over many years, used with both outpatients and inpatients, in an iterative manner to ensure maximum utility and user-experience. Most programs don't use this approach.

There are a small number of service developers trying to develop a digital mental health service for people with schizophrenia and schizoaffective disorder that use Autonomous learning theory principles but they are developing very narrow tools—so-called “cognitive remediation” programs—that cater very specifically to the cognitive dysfunction of these patients with very disabling chronic persistent psychotic disorders and not to other contributors to their dysfunction—so it is only trying to solve one problem amongst a multitude or cluster of dysfunctions occurring in these patients propagating their conditions—so this is an example of what would be termed as a subspecialized approach or solution, which is typical of mental health services especially digital mental health services—they all are quite narrow in scope because their underlying causation and formulatory models are narrow. However, the present disclosure is different as the present disclosure provides a unique over-arching model of psychopathology (mental disorders, dysfunction, disability) that no-one else has. This is done by programming or wiring the over-arching model of psychopathology into the categorizations of:

(1) normal ‘brain,’ mind,’ ‘brain/mind,’ ‘brain/mind-body’ and ‘brain/mind-body-external world interactions’ function, (in this model ‘brain’ and ‘mind’ is defined in quite a novel way, breaking each of their definitions into 3 elements);

(2) psychopathology (the types of brain dysfunction that can cause clusters of disorders such as the three clusters of personality disorders, but, in particular, the unique three formulatory subtypes model); and

(3) three formulatory subtypes of mental disorder model: can divide the cause of all mental disorders into Mind, Body and Brain mechanisms.

The adoption of a tripartite categorization system to mental health has proved unbelievably useful—see elsewhere: a recurring theme throughout the system of the present disclosure.

In the prior art, there is limited mental health online infrastructure to broker patients with in-house trained and accredited mentors, clinicians (diagnosticians, therapists) and coaches that also includes case conferencing. A true online brokerage program has not been developed—as opposed to a database of help-providers, because an online infrastructure has not been constructed that consists of the necessary ingredients:

Although the present disclosure has been described with reference to specific examples, it will be appreciated by those skilled in the art that the present disclosure may be embodied in many other forms, in keeping with the broad principles and the spirit of the present disclosure described herein.

The present disclosure and the described preferred embodiments specifically include at least one feature that is industrial applicable.

What is claimed is:

1. A system comprising a security system, one or more servers, and a platform for providing a plurality of digital mental health services to a user device,
wherein the digital mental health services comprising one or more of eLEARNING service, CONNECT services, and PLUS other additional services that working together function as a ‘virtual mental health system infrastructure.’

2. The system of claim 1, wherein the digital mental health services comprise eMental Health Literacy Education, Training, and Accreditation.

3. The system of claim 1, wherein the eLEARNING service is adapted to provide all user types in the one product, including self-help, help-seeking and peer support mentor consumers; mentor, clinician and coach help-providers for training, instant online support, and CPD services.

4. The system of claim 1, wherein the eLEARNING service incorporates autonomous learning principles with an innovative true visual learning approach with visual analogies, role play with conversing characters and avatars enabling a minimization of the use of technical words and jargon.

5. The system of claim 2, wherein the eLEARNING service incorporates continuous user interactivity, personalizing opportunities and embedded quizzes to ameliorate training pressure.

6. The system of claim 3, wherein the eLEARNING service incorporates novel psychiatry-grade ‘explanatory categorizations’ and a syllabus to complement and enhance recognized existing descriptive classifications.

7. The system of claim 4, wherein the eLEARNING service is adapted to supplement, complement and make more efficient and safe existing in-person general practitioner, specialist and allied health mental health care.

8. The system of claim 1 further comprising an AI engine for processing user feedback from the eLEARNING service and adjust an eLEARNING program based on the user feedback and aggregated data of other users.

9. The system of claim 1, wherein the digital mental health services is adapted to provide Online access for supplementary and complementary services to existing primary and specialist healthcare that provide access to cutting edge interventions and technological developments, such as artificial intelligence, Virtual Reality, neurostimulation therapies.

10. The system of claim 1, wherein the user device comprises a processor, a human user interface, a machine-to-machine interface and a network device, herein the human user interface is adapted to deliver a product generated by an AI engine, the human user interface is adapted to deliver a virtual reality environment, and the machine-to-machine interface is adapted to connect to a medical device to deliver neurostimulation therapies.

11. The system of claim 10, wherein the network device is adapted to connect to the digital mental health services where telecommunication health consultations services can be delivered.

12. The system of claim 11, wherein the virtual reality environment comprises a Virtual Hospital-Community Mental Health Care environment with a specialized psychiatric online virtual healthcare system infrastructure.

13. The system of claim 12, wherein the specialized psychiatric online virtual healthcare system infrastructure is adapted to provide clinical care pathways that are supplementary and complementary to existing in-person care, incorporating multi-modal, trans-diagnostic, multi-user type services, and has hospital-grade healthcare standards, regulations and accreditation processes to maintain quality control for all users.
